(19) United States
(12) Patent Application Publication
(10) Pub. No.: US 2022/0287836 A1
(43) Pub. Date: Sep. 15, 2022

(54) SYSTEMS AND METHODS FOR TRICUSPID VALVE TREATMENT

(71) Applicant: Edwards Lifesciences Corporation, Irvine, CA (US)

(72) Inventors: David Robert Landon, Huntington Beach, CA (US); Taylor Jacob Scheinblum, Newport Beach, CA (US); Matthew Michael Becerra, Lake Forest, CA (US); Alexander H. Cooper, Costa Mesa, CA (US); Jesse Robert Edwards, Silverado, CA (US); Hieu Minh Luong, Westminster, CA (US); David L. Hauser, Newport Beach, CA (US); Pui Tong Ho, Ladera Ranch, CA (US)

(73) Assignee: Edwards Lifesciences Corporation, Irvine, CA (US)

(21) Appl. No.: 17/681,660

(22) Filed: Feb. 25, 2022

Related U.S. Application Data
(63) Continuation of application No. PCT/US2020/054786, filed on Oct. 8, 2020.
(60) Provisional application No. 62/925,027, filed on Oct. 23, 2019.

(51) Int. Cl. A61F 2/24; A61F 2/95

(52) U.S. Cl. CPC A61F 2/2436; A61F 2/2418; A61F 2/2439; A61F 2/9517; A61F 2/2466

(57) ABSTRACT

Devices, systems and methods are described herein to provide improved treatment of a tricuspid valve. Such treatment may include tricuspid valve replacement, which may include providing a prosthetic tricuspid valve within the tricuspid valve annulus. Delivery systems for delivering the prosthetic tricuspid valve to the tricuspid valve annulus are disclosed herein. Treatment may also include repair of the tricuspid valve.

### CROSS REFERENCE TO RELATED APPLICATIONS

This application is a continuation of International Application No. PCT/US2020/054786, filed Oct. 8, 2020, which designates the United States and was published in English by the International Bureau on Apr. 29, 2021 as WO2021/080782, which claims priority to U.S. Provisional App. No. 62/925,027, filed Oct. 23, 2019, the entirety of which is hereby incorporated by reference.

### BACKGROUND

### Field

Certain embodiments disclosed herein relate generally to prostheses for implantation within a lumen or body cavity and delivery systems for a prosthesis. In particular, the prostheses and delivery systems relate in some embodiments to replacement heart valves, such as replacement tricuspid heart valves.

### Background

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner.

Delivering a prosthesis to a desired location in the human body, for example delivering a replacement heart valve to the tricuspid valve, can also be challenging. Obtaining access to perform procedures in the heart or in other anatomical locations may require delivery of devices percutaneously through tortuous vasculature or through open or semi-open surgical procedures. The ability to control the deployment of the prosthesis at the desired location can also be challenging.

### SUMMARY

Embodiments of the present disclosure are directed to a prosthesis, such as but not limited to a replacement heart valve. Embodiments of the present disclosure may also be directed to delivery systems, devices and/or methods of use to deliver and/or controllably deploy a prosthesis, such as but not limited to a replacement heart valve, to a desired location within the body. In some embodiments, a replacement heart valve and methods for delivering a replacement heart valve to a native heart valve, such as a tricuspid valve, are provided.

In some embodiments, a delivery system and method are provided for delivering a replacement heart valve to a native tricuspid valve location. In some embodiments, components of the delivery system facilitate bending of the delivery system to steer a prosthesis within a right atrium to a location within the native tricuspid valve. In some embodiments, a capsule is provided for containing the prosthesis for delivery to the native tricuspid valve location. In other embodiments, the delivery system and method may be adapted for delivery of implants to locations other than the native tricuspid valve.

The present disclosure includes, but is not limited to, the following embodiments.

A delivery system for an implant, the delivery system including an elongate shaft having a distal end, an implant retention area for retaining the implant, a bend portion configured to deflect the distal end of the elongate shaft to a first direction, and a portion positioned proximal of the bend portion. A deflection mechanism is configured to deflect the portion that is positioned proximal of the bend portion to deflect the bend portion towards a second direction that is opposed to the first direction.

A delivery system for an implant, the delivery system including an elongate shaft having a distal end, an implant retention area for retaining the implant, a bend portion configured to deflect the distal end of the elongate shaft in a first plane, and a portion positioned proximal of the bend portion. A deflection mechanism is configured to deflect the portion that is positioned proximal of the bend portion in one or more planes that are not perpendicular to the first plane.

A delivery system for an implant, the delivery system including an elongate shaft having a distal end, an implant retention area for retaining the implant, a first bend portion configured to deflect the distal end of the elongate shaft to a first direction, a second bend portion positioned proximate of the first bend portion and configured to deflect the distal end of the elongate shaft to a second direction, and a portion positioned proximal of the second bend portion. A deflection mechanism may be configured to deflect the first bend portion and the second bend portion and the portion that is positioned proximal of the second bend portion.

A delivery system for an implant, the delivery system including an elongate shaft having an implant retention area for retaining the implant, and a capsule having a distal end and surrounding the implant retention area, and the distal end of the capsule forming a distal tip of the elongate shaft.

A delivery system for an implant, the delivery system including an elongate shaft having an implant retention area for retaining the implant, and a distal tip including a flexible sheath extending distally and configured to bend about a portion of a guide wire.

A delivery system for an implant, the delivery system including an elongate shaft having an implant retention area for retaining the implant, and a distal tip having a dome shape or a parabolic shape.

A delivery system for an implant, the delivery system including an elongate shaft having a wall surrounding a channel for the implant to be passed through for deployment of the implant, the wall configured to have a bend defining a bend in the channel during deployment of the implant.

A delivery system for an implant, the delivery system including an elongate shaft having an axial dimension and having an implant retention area for retaining the implant, and a port for the implant to be deployed from the elongate shaft in a direction transverse to the axial dimension.

A delivery system for an implant, the delivery system including an elongate shaft having an implant retention area for retaining the implant, the elongate shaft configured to bend more than 180 degrees to form a loop.

A delivery system for an implant, the delivery system including an elongate shaft having a capsule surrounding an implant retention area for retaining the implant, and a hinge coupling the capsule to a portion of the elongate shaft.

A delivery system for an implant, the delivery system including an elongate shaft extending along an axis and having an outer surface and an implant retention area for retaining the implant. One or more support bodies may be configured to extend radially outward from the outer surface of the elongate shaft and contact an external surface to resist deflection of the elongate shaft transverse to the axis.

A system including a prosthetic heart valve configured for implantation within a patient's valve annulus. The system includes an anchor configured to be secured within a portion of the patient's body. The system includes a tether configured to couple the prosthetic heart valve to the anchor.

A prosthetic valve for replacement of a patient's native valve, the prosthetic valve including a prosthetic heart valve body configured to be anchored within an annulus of the patient's native valve and forming a prosthetic valve annulus. The system includes a port coupled to the prosthetic heart valve body and configured to receive a diagnostic or therapeutic device.

A method for treating a patient's tricuspid valve, the method including passing a delivery apparatus for an implant into the patient's right atrium. The method including deploying the implant to the patient's tricuspid valve.

A method for treating a patient's tricuspid valve, the method including deploying a prosthetic heart valve within a patient's tricuspid valve annulus. The method including deploying an anchor to a portion within a patient's body. The method including providing a tether coupling the prosthetic heart valve to the anchor.

A method including passing a diagnostic or therapeutic device through a port positioned on a prosthetic heart valve body, the prosthetic heart valve body forming a prosthetic valve annulus.

A method including coupling a pacemaker pacing lead to a prosthetic heart valve body positioned within a patient's heart valve annulus to provide electrical energy through the pacemaker pacing lead and through the prosthetic heart valve body to pace functioning of the patient's heart.

A method including delivering a delivery apparatus for an implant into a portion of a patient's heart, the delivery apparatus including an elongate shaft extending along an axis and having an outer surface. The method including expanding one or more support bodies radially outward from the outer surface of the elongate shaft. The method including contacting the one or more support bodies to a surface external of the delivery apparatus to resist deflection of the elongate shaft transverse to the axis.

### BRIEF DESCRIPTION OF THE DRAWINGS

FIG. 1 shows an embodiment of a delivery system.

FIG. 2A shows a partial cross-sectional view of the distal end of the delivery system of FIG. 1 loaded with the valve prosthesis of FIG. 3A.

FIG. 2B shows a partial cross-sectional view of the distal end of the delivery system of FIG. 1 without the valve prosthesis of FIG. 3A.

FIG. 2C shows a partial cross-sectional view of the distal end of the delivery system of FIG. 1 with certain shaft assemblies translated along the rail assembly.

FIG. 3A shows a side view of an embodiment of a valve prosthesis that may be delivered using the delivery systems described herein.

FIG. 3B shows a side view of an embodiment of an aortic valve prosthesis that may be delivered using the delivery systems described herein.

FIG. 4 shows a perspective view of the distal end of the delivery system of FIG. 1.

FIG. 5 shows components of the delivery system of FIG. 4 with the outer sheath assembly moved proximally and out of view.

FIG. 6A shows components of the delivery system of FIG. 5 with the mid shaft assembly moved proximally and out of view.

FIG. 6B illustrates a cross-section of the rail assembly.

FIG. 6C illustrates a cross-section of an embodiment of the rail assembly.

FIG. 7 shows components of a delivery system.

FIG. 8 shows components of the delivery system of FIG. 7 with the inner assembly moved proximally and out of view.

FIG. 9 illustrates an embodiment of a rail assembly.

FIG. 10 illustrates an embodiment of a delivery system handle.

FIG. 11 illustrates a cross-section of the delivery system handle of FIG. 10.

FIG. 12A illustrates a side view of a distal end of an elongate shaft.

FIG. 12B illustrates a side view of the distal end of the elongate shaft deflected from the position shown in FIG. 12A.

FIG. 12C illustrate a top view of the distal end of the elongate shaft deflected from the position shown in FIG. 12A.

FIG. 13A illustrates a side view of a distal end of an elongate shaft.

FIG. 13B illustrates a side view of the distal end of the elongate shaft deflected from the position shown in FIG. 13A.

FIG. 13C illustrate a top view of the distal end of the elongate shaft deflected from the position shown in FIG. 13A.

FIG. 13D illustrates a front view of the distal end of the elongate shaft deflected from the position shown in FIG. 13A.

FIG. 14A illustrates a perspective view of a deflection mechanism positioned upon an elongate shaft.

FIG. 14B illustrates a side view of a distal end of an elongate shaft.

FIG. 14C illustrates a side view of the distal end of the elongate shaft deflected from the position shown in FIG. 14B.

FIG. 14D illustrate a top view of the distal end of the elongate shaft deflected from the position shown in FIG. 14B.

FIG. 15A illustrates a side view of a distal end of an elongate shaft.

FIG. 15B illustrates a side view of the distal end of the elongate shaft deflected from the position shown in FIG. 15A.

FIG. 15C illustrate a top view of the distal end of the elongate shaft deflected from the position shown in FIG. 15A.

FIG. 16A illustrates a side view of a distal end of an elongate shaft.

FIG. 16B illustrates a side view of the distal end of the elongate shaft deflected from the position shown in FIG. 16A.

FIG. 16C illustrate a top view of the distal end of the elongate shaft deflected from the position shown in FIG. 16A.

FIG. 17 illustrates a perspective view of a rail assembly having a pull tether positioned thereon.

FIG. 18A illustrates a perspective view of a rail assembly having cuts positioned on a tube of the rail assembly.

FIG. 18B illustrates a cross sectional view of the rail assembly having stoppers positioned therein.

FIG. 19A illustrates a side view of the distal end of the elongate shaft.

FIG. 19B illustrate a top view of the distal end of the elongate shaft shown in FIG. 19A.

FIG. 20A illustrates a representation of an elongate shaft entering a right atrium of a patient's heart.

FIG. 20B illustrates a distal end of the elongate shaft shown in FIG. 20A being deflected from the position shown in FIG. 20A.

FIG. 20C illustrates a distal end of the elongate shaft shown in FIG. 20B being deflected from the position shown in FIG. 20B.

FIG. 21 illustrates a representation of an elongate shaft entering a right atrium of a patient's heart from the superior vena cava.

FIG. 22A illustrates a perspective view of an implant being deployed from an elongate shaft.

FIG. 22B illustrates a perspective view of an implant being deployed from an elongate shaft.

FIG. 22C illustrates a perspective view of an implant being deployed from an elongate shaft.

FIG. 23 illustrates an implant in position within a tricuspid valve annulus.

FIG. 24 illustrates an embodiment of a tip of an elongate shaft.

FIG. 25A illustrates an embodiment of a tip of an elongate shaft.

FIG. 25B illustrates a view of a capsule being positioned within a right ventricle of a patient's heart.

FIG. 26 illustrates an embodiment of a tip of an elongate shaft.

FIG. 27 illustrates an embodiment of a tip of an elongate shaft.

FIG. 28 illustrates an embodiment of a tip of an elongate shaft.

FIG. 29 illustrates a view of a flexible implant being positioned within an elongate shaft having a bend.

FIG. 30 illustrates a view of an implant configured to be deployed from a port in a side of an elongate shaft.

FIG. 31 illustrates a view of an elongate shaft having a loop.

FIG. 32A illustrates a view of an elongate shaft having a hinge.

FIG. 32B illustrates a view of the elongate shaft shown in FIG. 32A with a capsule rotated from the position shown in FIG. 32A.

FIG. 33A illustrates a view of an elongate shaft having a hinge.

FIG. 33B illustrates a view of the elongate shaft shown in FIG. 33A with a capsule rotated from the position shown in FIG. 33A.

FIG. 34A illustrates a view of an elongate shaft within a patient's right atrium.

FIG. 34B illustrates a view of the elongate shaft shown in FIG. 34A translated from the position shown in FIG. 34A.

FIG. 35 illustrates a view of an implant within a patient's tricuspid valve annulus with an anchor positioned in the inferior vena cava.

FIG. 36A illustrates a view of an implant within a patient's tricuspid valve annulus with an anchor coupled to the moderator band of the right ventricle.

FIG. 36B illustrates a perspective view of an anchor for coupling to the moderator band.

FIG. 36C illustrates a perspective view of an anchor for coupling to the moderator band.

FIG. 36D illustrates a perspective view of an anchor for coupling to the moderator band.

FIG. 36E illustrates a perspective view of an anchor for coupling to the moderator band.

FIG. 36F illustrates a perspective view of an anchor for coupling to the moderator band.

FIG. 37 illustrates a view of an implant within a patient's tricuspid valve annulus with an anchor coupled to a wall of the right ventricle.

FIG. 38A illustrates a view of an implant within a patient's right atrium with an anchor coupled to a wall of the right ventricle.

FIG. 38B illustrates a view of the implant of FIG. 38A within the patient's tricuspid valve annulus.

FIG. 39A illustrates a side schematic view of an implant including a port for receiving a diagnostic or therapeutic device.

FIG. 39B illustrates a side schematic view of an implant including a port for receiving a diagnostic or therapeutic device.

FIG. 39C illustrates a side perspective view of an implant including a port for receiving a diagnostic or therapeutic device.

FIG. 39D illustrates a bottom view of an implant including a port for receiving a diagnostic or therapeutic device.

FIG. 39E illustrates a side perspective view of a port for receiving a diagnostic or therapeutic device.

FIG. 39F illustrates a side perspective view of a port for receiving a diagnostic or therapeutic device.

FIG. 39G illustrates a side perspective view of a port for receiving a diagnostic or therapeutic device.

FIG. 40A illustrates a side schematic view of an implant including a port for receiving a diagnostic or therapeutic device.

FIG. 40B illustrates a side perspective view of an implant including a port for receiving a diagnostic or therapeutic device.

FIG. 41 illustrates a side perspective view of an implant including a port for receiving a diagnostic or therapeutic device.

FIG. 42 illustrates a view of an implant including a port for receiving a diagnostic or therapeutic device in position within the tricuspid valve annulus.

FIG. 43 illustrates a view of an implant including a port for receiving a diagnostic or therapeutic device in position within the tricuspid valve annulus.

FIG. 44 illustrates a view of an implant including a port for coupling to a pacemaker pacing lead.

FIG. 45 illustrates a perspective view of a delivery system.

FIG. 46 illustrates a schematic view of the handle of the delivery system shown in FIG. 45.

FIG. 47 illustrates a front plan view of an embodiment of an adaptor.

FIG. 48 illustrates a side perspective view of an embodiment of an adaptor and drive rods.

FIG. 49 illustrates a perspective view of the handle shown in FIG. 45.

FIG. 50 illustrates a perspective view of a proximal portion of the handle shown in FIG. 45.

FIG. 51 illustrates a side perspective view of insertion of a delivery apparatus into a patient's body.

FIG. 52 illustrates a perspective view of an embodiment of a distal end of an elongate sheath.

FIG. 53 illustrates a perspective view of an embodiment of a distal end of an elongate sheath.

FIG. 54 illustrates a cross sectional view of a capsule of the elongate sheath shown in FIG. 53.

FIG. 55 illustrates a side schematic view of the elongate sheath shown in FIG. 53 deploying an implant to a tricuspid heart valve.

FIG. 56 illustrates a view of an elongate shaft approaching a tricuspid valve.

FIG. 57 illustrates a view of the elongate shaft shown in FIG. 56 deflected in position.

FIG. 58 illustrates a view of an implant deployed to the tricuspid valve.

FIG. 59 illustrates a perspective view of an embodiment of a control device and an output device.

FIG. 60 illustrates a perspective view of an embodiment of a control device and an output device.

FIG. 61 illustrates a cross section view of an embodiment of a delivery system handle.

FIG. 62A illustrates a side view of a distal end of an elongate shaft.

FIG. 62B illustrates a side view of the elongate shaft shown in FIG. 62A, with support bodies deployed.

FIG. 62C illustrates a view of the elongate shaft shown in FIG. 62A approaching a mitral valve.

FIG. 62D illustrates a view of the elongate shaft shown in FIG. 62A approaching a mitral valve.

FIG. 62E illustrates a view of the elongate shaft shown in FIG. 62A approaching a mitral valve, with support bodies deployed.

FIG. 62F illustrates a view of an elongate shaft approaching a tricuspid valve, with support bodies deployed.

FIG. 63A illustrates a view of an elongate shaft approaching a mitral valve.

FIG. 63B illustrates a view of the elongate shaft shown in FIG. 63A approaching a mitral valve, with a support body deployed.

FIG. 64A illustrates a perspective view of a support body.

FIG. 64B illustrates a view of an elongate shaft approaching a mitral valve.

FIG. 64C illustrates a view of the elongate shaft shown in FIG. 64B approaching a mitral valve, with a support body deployed.

### DETAILED DESCRIPTION

The present specification and drawings provide aspects and features of the disclosure in the context of several embodiments of replacement heart valves, delivery systems and methods that are configured for use in the vasculature of a patient, such as for replacement or repair of natural heart valves in a patient. These embodiments may be discussed in connection with replacing specific valves such as the patient's aortic, tricuspid, mitral, or pulmonary valve. However, it is to be understood that the features and concepts discussed herein can be applied to devices other than heart valve implants. For example, the delivery systems, replacement heart valves, and methods can be applied to medical implants, for example other types of expandable prostheses, for use elsewhere in the body, such as within an artery, a vein, or other body cavities or locations. In addition, specific features of a valve, delivery system, method, etc. should not be taken as limiting, and features of any one embodiment discussed herein can be combined with features of other embodiments as desired and when appropriate. While certain of the embodiments described herein are described in connection with a transfemoral delivery approach, it should be understood that these embodiments can be used for other delivery approaches such as, for example, transapical, transatrial, or transjugular approaches. Moreover, it should be understood that certain of the features described in connection with certain embodiments can be incorporated with other embodiments, including those that are described in connection with different delivery approaches.

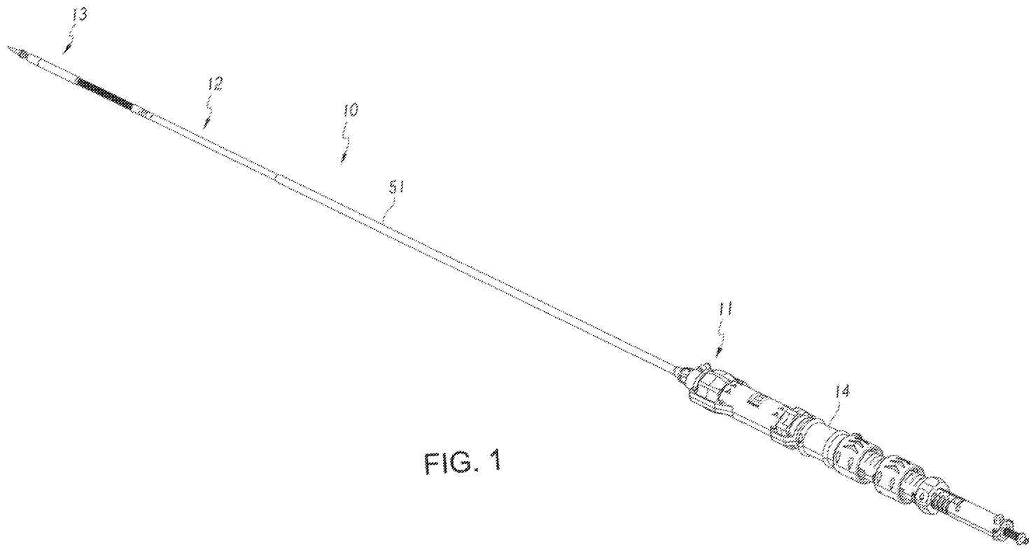

FIG. 1 illustrates an embodiment of a delivery device, assembly, or system 10. The delivery system 10 can be used to deploy a prosthesis, such as a replacement heart valve, within the body. In some embodiments, the delivery system 10 can use a dual plane deflection approach to properly deliver the prosthesis. Replacement heart valves can be delivered to a patient's tricuspid heart valve annulus or other heart valve location in various manners, such as by open surgery, minimally-invasive surgery, and percutaneous or transcatheter delivery through the patient's vasculature. Example transfemoral approaches may be found in U.S. Pat. Pub. No. 2015/0238315, filed Feb. 20, 2015, the entirety of which is hereby incorporated by reference in its entirety. While the delivery system 10 is described in connection with a percutaneous delivery approach, and more specifically a transfemoral delivery approach, it should be understood that features of delivery system 10 can be applied to other delivery system, including delivery systems for a transapical, transatrial, or transjugular delivery approach.

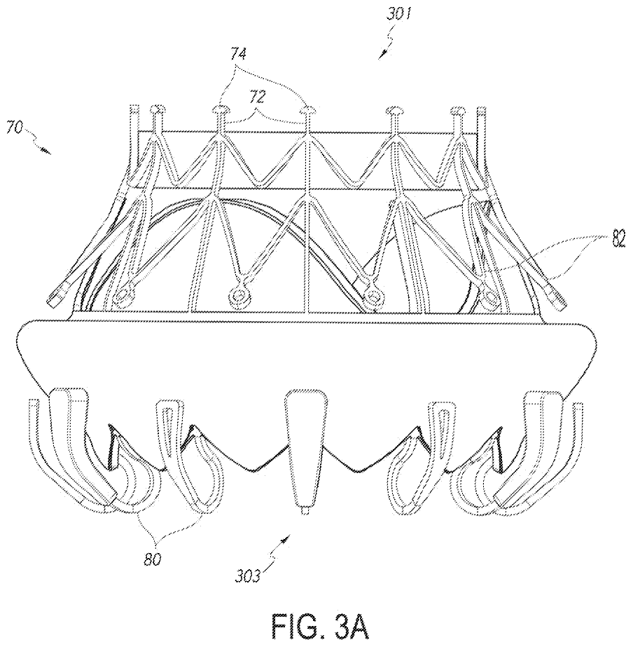

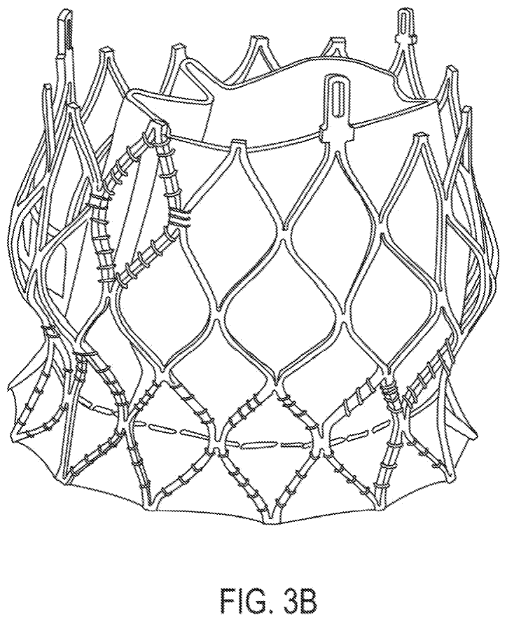

The delivery system 10 may be used to deploy a prosthesis, such as a replacement heart valve as described elsewhere in this specification, within the body. The delivery system 10 can receive and/or cover portions of the prosthesis such as a first end 301 and second end 303 of the prosthesis or implant 70 illustrated in FIG. 3A. For example, the delivery system 10 may be used to deliver an expandable prosthesis or implant 70, where the implant 70 includes the first end 301 and the second end 303, and wherein the second end 303 is configured to be deployed or expanded before the first end 301.

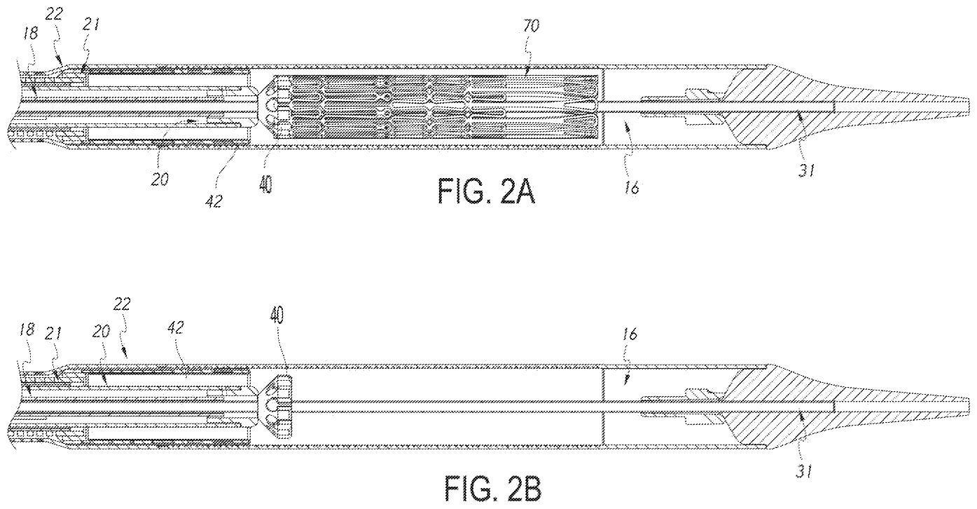

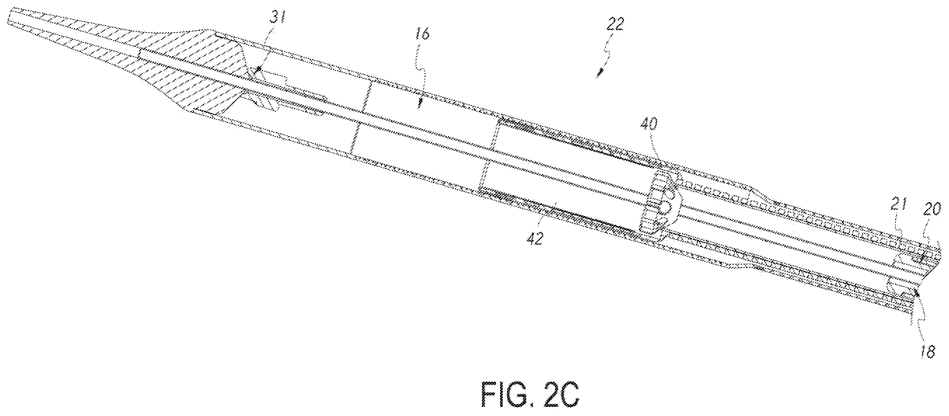

FIG. 2A further shows an example of the implant 70 that can be inserted into the delivery system 10, specifically into the implant retention area 16. For ease of understanding, in FIG. 2A, the prosthesis is shown with only the bare metal frame illustrated. The prosthesis or implant 70 can take any number of different forms. A particular example of frame for a prosthesis is shown in FIG. 3A, though it will be understood that other designs and frame configurations may also be used, including those disclosed in this application. The implant 70 can include one or more sets of anchors, such as distal (or ventricular) anchors 80 extending proximally when the prosthesis frame is in an expanded configuration and proximal (or atrial) anchors 82 extending distally when the prosthesis frame is in an expanded configuration. The prosthesis can further include struts 72 which may end in mushroom-shaped tabs 74 at the first end 301. Further discussion can be found in U.S. Publication No. 2015/0328000A1, published Nov. 19, 2015, hereby incorporated by reference in its entirety.

In some embodiments, the delivery system 10 can be used in conjunction with a replacement aortic valve, such as shown in FIG. 3B. In some embodiments the delivery system 10 can be modified to support and delivery the replacement aortic valve. However, the procedures and structures discussed below can similarly be used for a replacement tricuspid and replacement aortic valve.

Additional details and example designs for a prosthesis are described in U.S. Pat. Nos. 8,403,983, 8,414,644, 8,652,203 and U.S. Patent Publication Nos. 2011/0313515, 2012/0215303, 2014/0277390, 2014/0277422, 2014/0277427, 2018/0021129, and 2018/0055629, the entirety of these patents and publications are hereby incorporated by reference and made a part of this specification. Further details and embodiments of a replacement heart valve or prosthesis and its method of implantation are described in U.S. Publication Nos. 2015/0328000 and 2016/0317301 the entirety of each of which is hereby incorporated by reference and made a part of this specification.

The delivery system 10 can be relatively flexible. In some embodiments, the delivery system 10 is particularly suitable for delivering a replacement heart valve to a mitral valve location through a transseptal approach (e.g., between the right atrium and left atrium via a transseptal puncture). The delivery system 10, however, may be suitable for delivering a replacement heart valve to a tricuspid valve location, among other locations.

As shown in FIG. 1, the delivery system 10 can include a shaft assembly or elongate shaft 12 comprising a proximal end 11 and a distal end 13, wherein a handle 14 is coupled to the proximal end of the elongate shaft 12. The elongate shaft 12 can be used to hold the implant 70 for advancement of the same through the vasculature to a treatment location. The delivery system 10 can further comprise a relatively rigid live-on (or integrated) sheath 51 surrounding the elongate shaft 12 that can prevent unwanted motion of the elongate shaft 12. The live-on sheath 51 can be attached at a proximal end of elongate shaft 12 proximal to the handle 14, for example at a sheath hub. The elongate shaft 12 can include an implant retention area 16 (shown in FIGS. 2A-2B with FIG. 2A showing the implant 70 and FIG. 2B with the implant 70 removed) at its distal end that can be used for this purpose. In some embodiments, the elongate shaft 12 can hold an expandable prosthesis in a compressed state at implant retention area 16 for advancement of the implant 70 within the body. The elongate shaft 12 may then be used to allow controlled expansion of the implant 70 at the treatment location. In some embodiments, the elongate shaft 12 may be used to allow for sequential controlled expansion of the implant 70 as discussed in detail below. The implant retention area 16 is shown in FIGS. 2A—B at the distal end of the delivery system 10, but may also be at other locations. In some embodiments, the implant 70 may be rotated in the implant retention area 16, such as through the rotation of the inner shaft assembly 18 discussed herein.

As shown in cross-sectional view of FIGS. 2A-2B, the distal end of the delivery system 10 can include one or more subassemblies such as an outer sheath assembly 22, a mid shaft assembly 21, a rail assembly 20, an inner shaft assembly 18, and a nose cone assembly 31 as will be described in more detail below. In some embodiments, the delivery system 10 may not have all of the assemblies disclosed herein. For example, in some embodiments a full mid shaft assembly may not be incorporated into the delivery system 10. In some embodiments, the assemblies disclosed below may be in a different radial order than is discussed.

In particular, embodiments of the disclosed delivery system 10 can utilize a steerable rail in the rail assembly 20 for steering the distal end of the delivery system 10, allowing the implant to be properly located in a patient's body. As discussed in detail below, the steerable rail can be, for example, a rail shaft that extends through the delivery system 10 from the handle 14 generally to the distal end. In some embodiments, the steerable rail has a distal end that ends proximal to the implant retention area 16. A user can manipulate the bending of the distal end of the rail, thereby bending the rail in a particular direction. In preferred embodiments, the rail has more than one bend along its length, thereby providing multiple directions of bending. As the rail is bent, it presses against the other assemblies to bend them as well, and thus the other assemblies of the delivery system 10 can be configured to steer along with the rail as a cooperating single unit, thus providing for full steerability of the distal end of the delivery system.

Once the rail is steered into a particular location in a patient's body, the implant 70 can be advanced along or relative to the rail through the movement of the other sheaths/shafts relative to the rail and released into the body. For example, the rail can be bent into a desired position within the body, such as to direct the implant 70 towards the native mitral valve. The other assemblies (e.g., the outer sheath assembly 22, the mid shaft assembly 21, the inner assembly 18, and the nose cone assembly 31) can passively follow the bends of the rail. Further, the other assemblies (e.g., the outer sheath assembly 22, the mid shaft assembly 21, the inner assembly 18, and the nose cone assembly 31) can be advanced together (e.g., relatively together, sequentially with one actuator, simultaneously, almost simultaneously, at the same time, closely at the same time) relative to the rail while maintaining the implant 70 in the compressed position without releasing or expanding the implant 70 (e.g., within the implant retention area 16). The other assemblies (e.g., the outer sheath assembly 22, the mid shaft assembly 21, the inner assembly 18, and the nose cone assembly 31) can be advanced distally or proximally together relative to the rail. In some embodiments, only the outer sheath assembly 22, mid shaft assembly 21, and inner assembly 18 are advanced together over the rail. Thus, the nose cone assembly 31 may remain in the same position. The assemblies can be individually, sequentially, or simultaneously, translated relative to the inner assembly 18 in order to release the implant 70 from the implant retention area 16.

FIG. 2C illustrates the sheath assemblies, specifically the outer sheath assembly 22, the mid shaft assembly 21, the inner shaft assembly 18, and the nose cone assembly 31 having translated distally together along the rail assembly 20, further details on the assemblies are below. In some embodiments, the outer sheath assembly 22, the mid shaft assembly 21, the inner shaft assembly 18, and the nose cone assembly 31 translate together (e.g., relatively together, sequentially with one actuator, simultaneously, almost simultaneously, at the same time, closely at the same time). This distal translation can occur while the implant 70 remains in a compressed configuration within the implant retention area 16.

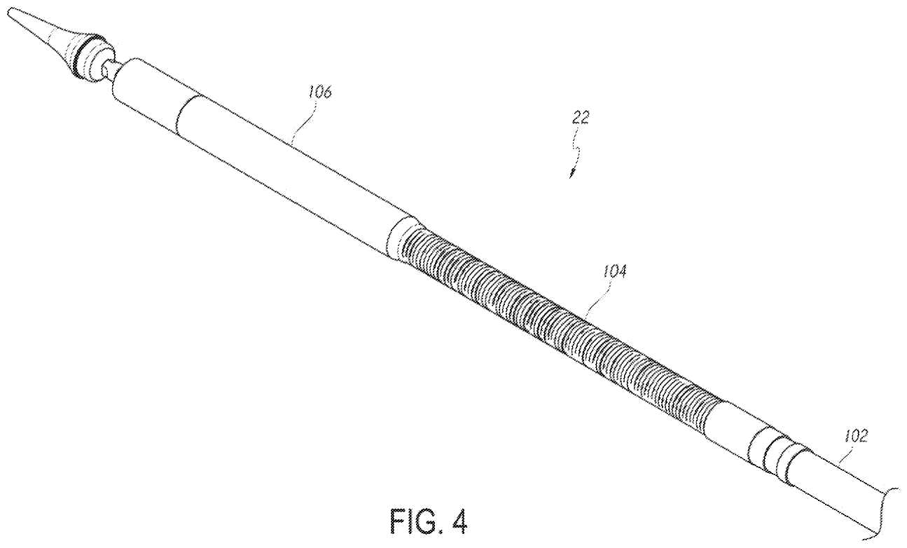

As shown in FIGS. 2A-2C and as further shown in FIGS. 4-8, starting with the outermost assembly, the delivery system can include an outer sheath assembly 22 forming a radially outer covering, or sheath, to surround an implant retention area 16 and prevent the implant from radially expanding. Specifically, the outer sheath assembly 22 can prevent radial expansion of the distal end of the implant from radially expanding. Moving radially inward, the mid shaft assembly 21 can be composed of a mid shaft hypotube 43 with its distal end attached to an outer retention member or outer retention ring 42 for radially retaining a portion of the prosthesis in a compacted configuration, such as a proximal end of the implant 70. The mid shaft assembly 21 can be located within a lumen of the outer sheath assembly 22. Moving further inwards, the rail assembly 20 can be configured for steerability, as mentioned above and further described below. The rail assembly 20 can be located within a lumen of the mid shaft assembly 21. Moving further inwards, the inner shaft assembly 18 can be composed of an inner shaft with its distal end attached to inner retention member or inner retention ring 40 (such as a PEEK ring) for axially retaining the prosthesis, for example the proximal end of the prosthesis. The inner shaft assembly 18 can be located within a lumen of the rail assembly 20. Further, the most radially-inward assembly is the nose cone assembly 31 which includes the nose cone shaft 27 having its distal end connected to the nose cone 28. The nose cone 28 can have a tapered tip. The nose cone assembly 31 is preferably located within a lumen of the inner shaft assembly 18. The nose cone assembly 31 can include a lumen for a guide wire to pass therethrough.

The elongate shaft 12, and more specifically the nose cone assembly 31, inner assembly 18, rail assembly 20, mid shaft assembly 21, and outer sheath assembly 22, can be collectively configured to deliver an implant 70 positioned within the implant retention area 16 (shown in FIG. 2A) to a treatment location. One or more of the subassemblies can then be moved to allow the implant 70 to be released at the treatment location. For example, one or more of the subassemblies may be movable with respect to one or more of the other subassemblies. The handle 14 can include various control mechanisms that can be used to control the movement of the various subassemblies as will also be described in more detail below. In this way, the implant 70 can be controllably loaded onto the delivery system 10 and then later deployed within the body. Further, the handle 14 can provide steering to the rail assembly 20, providing for bending/flexing/steering of the distal end of the delivery system 10.

As will be discussed below, the inner retention member 40, the outer retention ring 42, and the outer sheath assembly 22 can cooperate to hold the implant 70 in a compacted configuration. The inner retention member 40 is shown engaging struts 72 at the proximal end 301 of the implant 70 in FIG. 2A. For example, slots located between radially extending teeth on the inner retention member 40 can receive and engage the struts 72 which may end in mushroom-shaped tabs 74 on the proximal end of the implant 70. The mid shaft assembly 21 can be positioned over the inner retention member 40 so that the first end 301 of the implant 70 is trapped between the inner retention member 40 and the outer retention ring 42, thereby securely attaching it to the delivery system 10 between the mid shaft assembly 21 and the inner retention member 40. The outer sheath assembly 22 can be positioned to cover the second end 303 of the implant 70.

The outer retention member 42 may be attached to a distal end of the mid shaft hypotube 43 which can in turn be attached to a proximal tube 44 at a proximal end, which in turn can be attached at a proximal end to the handle 14. The outer retention member 42 can provide further stability to the implant 70 when in the compressed position. The outer retention member 42 can be positioned over the inner retention member 40 so that the proximal end of the implant 70 is trapped therebetween, securely attaching it to the delivery system 10. The outer retention member 42 can encircle a portion of the implant 70, in particular the first end 301, thus preventing the implant 70 from expanding. Further, the mid shaft assembly 21 can be translated proximally with respect to the inner assembly 18 into the outer sheath assembly 22, thus exposing a first end 301 of the implant 70 held within the outer retention member 42. In this way the outer retention member 42 can be used to help secure an implant 70 to or release it from the delivery system 10. The outer retention member 42 can have a cylindrical or elongate tubular shape, and may be referred to as an outer retention ring, though the particular shape is not limiting.

As shown in FIG. 2A, the distal anchors 80 can be located in a delivered configuration where the distal anchors 80 point generally distally (as illustrated, axially away from the main body of the prosthesis frame and away from the handle of the delivery system). The distal anchors 80 can be restrained in this delivered configuration by the outer sheath assembly 22. Accordingly, when the outer sheath 22 is withdrawn proximally, the distal anchors 80 can flip positions (e.g., bend approximately 180 degrees) to a deployed configuration (e.g., pointing generally proximally). FIG. 2A also shows the proximal anchors 82 extending distally in their delivered configuration within the outer sheath assembly 22. In other embodiments, the distal anchors 80 can be held to point generally proximally in the delivered configuration and compressed against the body of the prosthesis frame.

The delivery system 10 may be provided to users with an implant 70 preinstalled. In other embodiments, the implant 70 can be loaded onto the delivery system shortly before use, such as by a physician or nurse.

FIGS. 4-8 illustrate further views of delivery system 10 with different assemblies translated proximally and described in detail.

Starting with the outermost assembly shown in FIG. 4, the outer sheath assembly 22 can include an outer proximal shaft 102 directly attached to the handle 14 at its proximal end and an outer hypotube 104 attached at its distal end. A capsule 106 can then be attached generally at the distal end of the outer hypotube 104. In some embodiments, the capsule 106 can be 28 French or less in size. These components of the outer sheath assembly 22 can form a lumen for the other subassemblies to pass through.

A capsule 106 can be located at a distal end of the outer proximal shaft 102. The capsule 106 can be a tube formed of a plastic or metal material. In some embodiments, the capsule 106 is formed of ePTFE or PTFE. In some embodiments, this capsule 106 is relatively thick to prevent tearing and to help maintain a self-expanding implant in a compacted configuration. In some embodiments the material of the capsule 106 is the same material as the coating on the outer hypotube 104. As shown, the capsule 106 can have a diameter larger than the outer hypotube 104, though in some embodiments the capsule 106 may have a similar diameter as the hypotube 104. In some embodiments, the capsule 106 may include a larger diameter distal portion and a smaller diameter proximal portion. In some embodiments, there may be a step or a taper between the two portions. The capsule 106 can be configured to retain the implant 70 in the compressed position within the capsule 106. Further construction details of the capsule 106 are discussed below.

The outer sheath assembly 22 is configured to be individually slidable with respect to the other assemblies. Further, the outer sheath assembly 22 can slide distally and proximally relative to the rail assembly 20 together with the mid shaft assembly 21, inner assembly 18, and nose cone assembly 31.

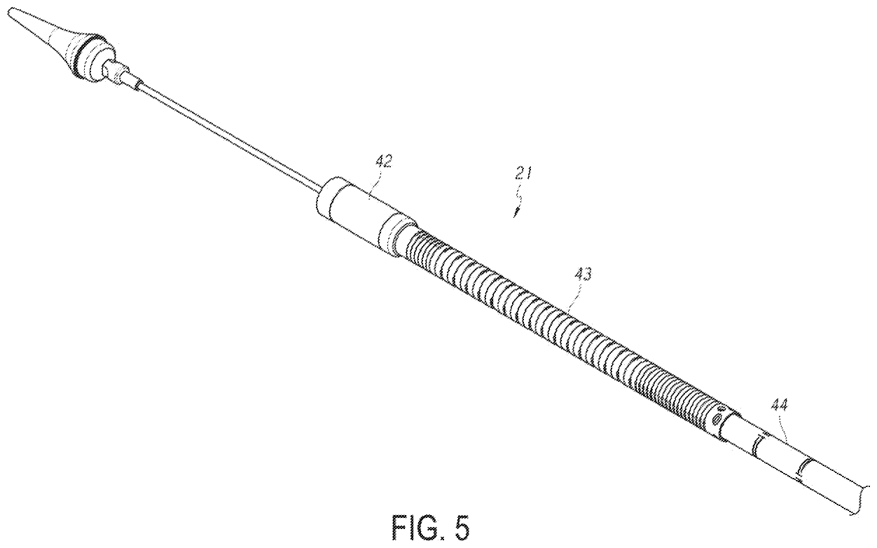

Moving radially inwardly, the next assembly is the mid shaft assembly 21. FIG. 5 shows a similar view as FIG. 4, but with the outer sheath assembly 22 removed, thereby exposing the mid shaft assembly 21.

The mid shaft assembly 21 can include a mid shaft hypotube 43 generally attached at its proximal end to a mid shaft proximal tube 44, which in turn can be attached at its proximal end to the handle 14, and an outer retention ring 42 located at the distal end of the mid shaft hypotube 43. Thus, the outer retention ring 42 can be attached generally at the distal end of the mid shaft hypotube 43. These components of the mid shaft assembly 21 can form a lumen for other subassemblies to pass through.

The outer retention ring 42 can be configured as a prosthesis retention mechanism that can be used to engage with the implant 70, as discussed with respect to FIG. 2A. For example, the outer retention ring 42 may be a ring or covering that is configured to radially cover the struts 72 on the implant 70. The outer retention ring 42 can also be considered to be part of the implant retention area 16, and may be at the proximal end of the implant retention area 16. With struts or other parts of an implant 70 engaged with the inner retention member 40, discussed below the outer retention ring 42 can cover both the implant 70 and the inner retention member 40 to secure the implant 70 on the delivery system 10. Thus, the implant 70 can be sandwiched between the inner retention member 40 of the inner shaft assembly 18 and the outer retention ring 42 of the mid shaft assembly 21.

The mid shaft assembly 21 is disposed so as to be individually slidable with respect to the other assemblies. Further, mid shaft assembly 21 can slide distally and proximally relative to the rail assembly 20 together with the outer sheath assembly 22, the inner assembly 18, and nose cone assembly 31.

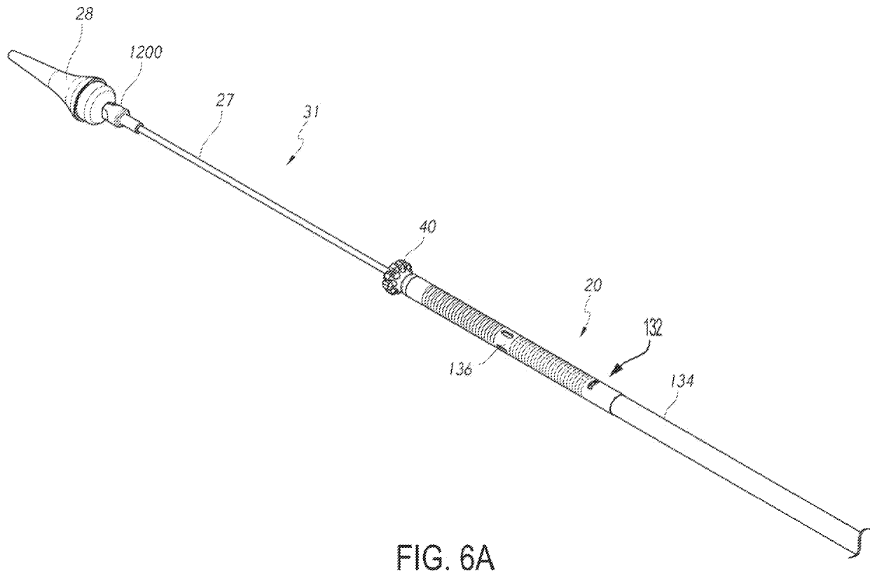

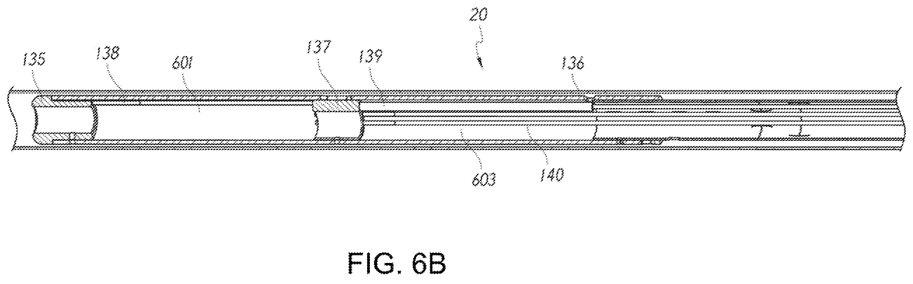

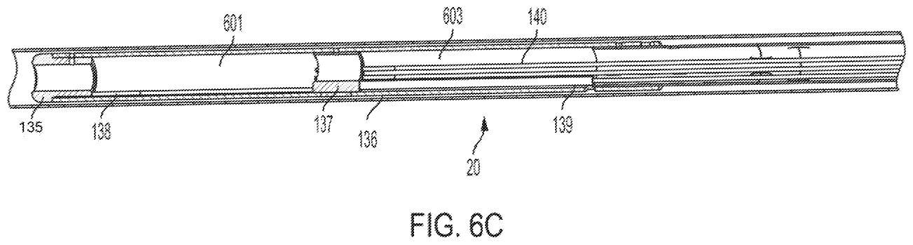

Next, radially inwardly of the mid shaft assembly 21 is the rail assembly 20. FIG. 6A shows approximately the same view as FIG. 5, but with the mid shaft assembly 21 removed, thereby exposing the rail assembly 20. FIG. 6B further shows a cross-section of the rail assembly 20 to view the pull wires. The rail assembly 20 can include a rail shaft 132 (or rail) generally attached at its proximal end to the handle 14. The rail shaft 132 can be made up of a rail proximal shaft 134 directly attached to the handle at a proximal end and a rail hypotube 136 attached to the distal end of the rail proximal shaft 134. The rail shaft 132 may include a proximal rail shaft portion 603 and a distal rail shaft portion 601. The rail hypotube 136 can further include an atraumatic rail tip at its distal end. Further, the distal end of the rail hypotube 136 can abut a proximal end of the inner retention member 40, as shown in FIG. 6A. In some embodiments, the distal end of the rail hypotube 136 can be spaced away from the inner retention member 40. These components of the rail shaft assembly 20 can form a lumen for the other subassemblies to pass through.

As shown in FIG. 6B, attached to an inner surface of the rail hypotube 136 are one or more pull wires which can be used apply forces to the rail hypotube 136 and steer the rail assembly 20. The pull wires can extend distally from the knobs in the handle 14, discussed below, to the rail hypotube 136. In some embodiments, pull wires can be attached at different longitudinal locations on the rail hypotube 136, thus providing for multiple bending locations in the rail hypotube 136, allowing for multidimensional steering.

In some embodiments, a distal pull wire 138 can extend to a distal section of the rail hypotube 136 and two proximal pull wires 140 can extend to a proximal section of the rail hypotube 136, however, other numbers of pull wires can be used, and the particular amount of pull wires is not limiting. For example, a two pull wires can extend to a distal location and a single pull wire can extend to a proximal location. In some embodiments, ring-like structures attached inside the rail hypotube 136, known as pull wire connectors, can be used as attachment locations for the pull wires, such as proximal ring 137 and distal ring 135. In some embodiments, the rail assembly 20 can include a distal pull wire connector 135 and a proximal pull wire connector 137. In some embodiments, the pull wires can directly connect to an inner surface of the rail hypotube 136.

The distal pull wire 138 can be connected (either on its own or through a connector 135) generally at the distal end of the rail hypotube 136. The proximal pull wires 140 can connect (either on its own or through a connector 137) at a location approximately one quarter, one third, or one half of the length up the rail hypotube 136 from the proximal end. In some embodiments, the distal pull wire 138 can pass through a small diameter pull wire lumen 139 (e.g., tube, hypotube, cylinder) attached on the inside of the rail hypotube 136. This can prevent the wires 138 from pulling on the rail hypotube 136 at a location proximal to the distal connection. Further, the lumen 139 can act as compression coils to strengthen the proximal portion of the rail hypotube 136 and prevent unwanted bending. Thus, in some embodiments the lumen 139 is only located on the proximal half of the rail hypotube 136. In some embodiments, multiple lumens 139, such as spaced longitudinally apart or adjacent, can be used per distal wire 138. In some embodiments, a single lumen 139 is used per distal wire 138. In some embodiments, the lumen 139 can extend into the distal half of the rail hypotube 136. In some embodiments, the lumen 139 is attached on an outer surface of the rail hypotube 136. In some embodiments, the lumen 139 is not used.

For the pair of proximal pull wires 140, the wires can be spaced approximately 180° from one another to allow for steering in both directions. Similarly, if a pair of distal pull wires 138 is used, the wires can be spaced approximately 180° from one another to allow for steering in both directions. In some embodiments, the pair of distal pull wires 138 and the pair of proximal pull wires 140 can be spaced approximately 90° from each other. In some embodiments, the pair of distal pull wires 138 and the pair of proximal pull wires 140 can be spaced approximately 0° from each other. However, other locations for the pull wires can be used as well, and the particular location of the pull wires is not limiting. In some embodiments, the distal pull wire 138 can pass through a lumen 139 attached within the lumen of the rail hypotube 136. This can prevent an axial force on the distal pull wire 138 from creating a bend in a proximal section of the rail hypotube 136.

FIG. 6C illustrates an embodiment in which the position of the proximal pull wires 140 has been moved 180° from the position shown in FIG. 6B. The position of the proximal pull wires 140 shown in FIG. 6C may allow the proximal portion of the rail hypotube 136 to bend in an opposite direction than the direction possible in FIG. 6B. For example, in the embodiment of FIG. 6B, when the distal portion of the rail hypotube 136 is deflected in a downward direction by the pull of the distal pull wires 138, the proximal portion of the rail hypotube 136 may be deflected leftward relative to the downward direction (viewing from the proximal end of the rail hypotube 136 toward the distal end of the rail hypotube 136). In the embodiment of FIG. 6C, however, when the distal portion of the rail hypotube 136 is deflected in a downward direction by the pull of the distal pull wires 138, the proximal portion of the rail hypotube 136 may be deflected rightward relative to the downward direction (viewing from the proximal end of the rail hypotube 136 toward the distal end of the rail hypotube 136). Such a variation may allow the proximal portion of the rail hypotube 136, and accordingly the elongate shaft 12 to deflect in an opposite direction than possible in the embodiment shown in FIG. 6B. The thickness of cuts on the rail shaft 132 may also be varied to allow for the opposite direction of deflection.

The rail assembly 20 is disposed so as to be slidable over the inner shaft assembly 18 and the nose cone assembly 31. In some embodiments, the outer sheath assembly 22, the mid shaft assembly 21, the inner shaft assembly 18, and the nose cone assembly 31 can be configured to slide together along or relative to the rail assembly 20, such as proximally and distally with or without any bending of the rail assembly 20. In some embodiments, the outer sheath assembly 22, the mid shaft assembly 21, the inner shaft assembly 18, and the nose cone assembly 31 can be configured to retain the implant 70 in a compressed position when they are simultaneously slid along or relative to the rail assembly 20.

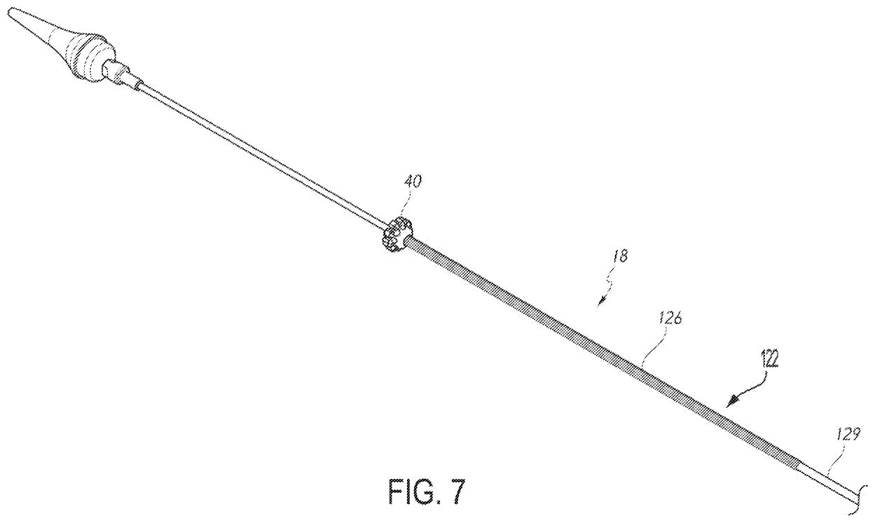

Moving radially inwards, the next assembly is the inner shaft assembly 18. FIG. 7 shows approximately the same view as FIG. 6A, but with the rail assembly 20 removed, thereby exposing the inner shaft assembly 18.

The inner shaft assembly 18 can include an inner shaft 122 generally attached at its proximal end to the handle 14, and an inner retention ring 40 located at the distal end of the inner shaft 122. The inner shaft 122 itself can be made up of an inner proximal shaft 129 directly attached to the handle 14 at a proximal end and a distal section 126 attached to the distal end of the inner proximal shaft 129. Thus, the inner retention ring 40 can be attached generally at the distal end of the distal section 126. These components of the inner shaft assembly 18 can form a lumen for the other subassemblies to pass through.

The inner retention member 40 can be configured as a prosthesis retention mechanism that can be used to engage with the implant 70, as discussed with respect to FIG. 2A. For example, the inner retention member 40 may be a ring and can include a plurality of slots configured to engage with struts 72 on the implant 70. The inner retention member 40 can also be considered to be part of the implant retention area 16, and may be at the proximal end of the implant retention area 16. With struts or other parts of an implant 70 engaged with the inner retention member 40, the outer retention ring 42 can cover both the prosthesis and the inner retention member 40 to secure the prosthesis on the delivery system 10. Thus, the implant 70 can be sandwiched between the inner retention member 40 of the inner shaft assembly 18 and the outer retention ring 42 of the mid shaft assembly 21.

The inner shaft assembly 18 is disposed so as to be individually slidable with respect to the other assemblies. Further, the inner assembly 18 can slide distally and proximally relative to the rail assembly 20 together with the outer sheath assembly 22, mid shaft assembly 21, and nose cone assembly 31.

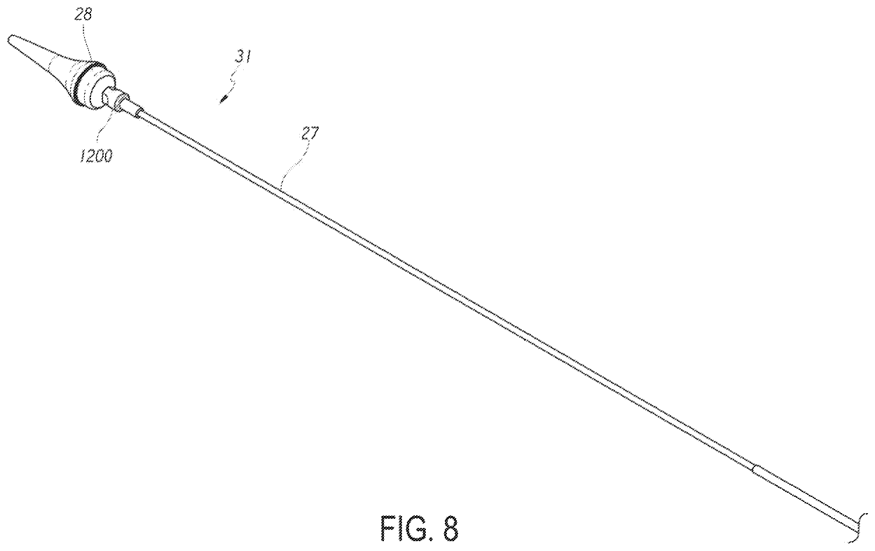

Moving further inwardly from the inner shaft assembly 18 is the nose cone assembly 31 also seen in FIG. 8. This may be a nose cone shaft 27, and in some embodiments, may have a nose cone 28 on its distal end. The nose cone 28 can be made of polyurethane for atraumatic entry and to minimize injury to venous vasculature. The nose cone 28 can also be radiopaque to provide for visibility under fluoroscopy.

The nose cone shaft 27 may include a lumen sized and configured to slidably accommodate a guide wire so that the delivery system 10 can be advanced over the guide wire through the vasculature. However, embodiments of the system 10 discussed herein may not use a guide wire and thus the nose cone shaft 27 can be solid. The nose cone shaft 27 may be connected from the nose cone 28 to the handle, or may be formed of different segments such as the other assemblies. Further, the nose cone shaft 27 can be formed of different materials, such as plastic or metal, similar to those described in detail above.

In some embodiments, the nose cone shaft 27 includes a guide wire shield 1200 located on a portion of the nose cone shaft 27.

The nose cone assembly 31 is disposed so as to be individually slidable with respect to the other assemblies. Further, the nose cone assembly 31 can slide distally and proximally relative to the rail assembly 20 together with the outer sheath assembly 22, mid shaft assembly 21, and inner assembly 18.

In some embodiments, one or more spacer sleeves (not shown) can be used between different assemblies of the delivery system 10. For example, a spacer sleeve can be located concentrically between the mid shaft assembly and the rail assembly 20, generally between the mid 43 and rail hypotubes 136. In some embodiments, the spacer sleeve can be generally embedded in the hypotube 43 of the mid shaft assembly 21, such as on an inner surface of the mid shaft assembly 21. In some embodiments, a spacer sleeve can be located concentrically between the rail assembly 20 and the inner assembly 18, generally within the rail hypotube 136. In some embodiments, a spacer sleeve can be used between the outer sheath assembly 22 and the mid shaft assembly 21. In some embodiments, a spacer sleeve can be used between the inner assembly 18 and the nose cone assembly 31. In some embodiments, 4, 3, 2, or 1 of the above-mentioned spacer sleeves can be used. The spacer sleeves can be used in any of the above positions.

As discussed above, the outer sheath assembly 22, the mid shaft assembly 21, the inner assembly 18, and the rail assembly 20 can contain an outer hypotube 104, a mid shaft hypotube, a distal section 126, and a rail hypotube 136, respectively. Each of these hypotubes/sections/shafts can be laser cut to include a number of slots, thereby creating a bending pathway for the delivery system to follow.

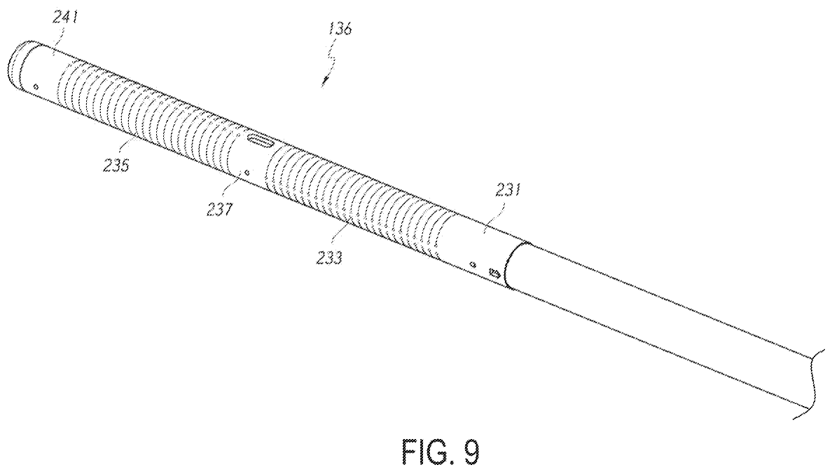

For example, FIG. 9 shows an embodiment of the rail hypotube 136. The rail hypotube 136 can also contain a number of circumferential slots. The rail hypotube 136 can generally be broken into a number of different sections. At the most proximal end is an uncut (or unslotted) hypotube section 231. Moving distally, the next section is the proximal slotted hypotube section 233. This section includes a number of circumferential slots cut into the rail hypotube 136. Generally, two slots are cut around each circumferential location forming almost half of the circumference. Accordingly, two backbones are formed between the slots extending up the length of the hypotube 136. This is the section that can be guided by the proximal pull wires 140. Moving further distally is the location 237 where the proximal pull wires 140 connect, and thus slots can be avoided. Thus section is just distal of the proximally slotted section.

Distally following the proximal pull wire connection area is the distal slotted hypotube section 235. This section is similar to the proximal slotted hypotube section 233, but has significantly more slots cut out in an equivalent length. Thus, the distally slotted hypotube section 235 provides easier bending than the proximally slotted hypotube section 233. The proximal and distal slotted hypotube sections 233, 235 may comprise bend portions of the rail shaft. In some embodiments, the proximal slotted section 233 can be configured to experience a bend of approximately 90 degrees with a half inch radius whereas the distal slotted section 235 can bend at approximately 180 degrees within a half inch. Further, as shown in FIG. 9, the spines of the distally slotted hypotube section 235 are offset from the spines of the proximally slotted hypotube section 233. Accordingly, the two sections will achieve different bend patterns, allowing for three-dimensional steering of the rail assembly 20. In some embodiments, the spines can be offset 30, 45, or 90 degrees, though the particular offset is not limiting. In some embodiments, the proximally slotted hypotube section 233 can include compression coils. This allows for the proximally slotted hypotube section 233 to retain rigidity for specific bending of the distally slotted hypotube section 235.

At the distalmost end of the distal slotted hypotube section 235 is the distal pull wire connection area 241 which is again a non-slotted section of the rail hypotube 136.

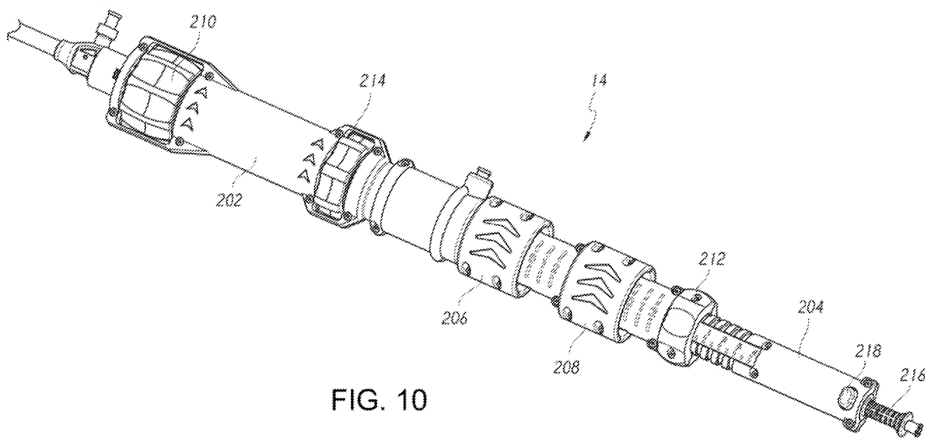

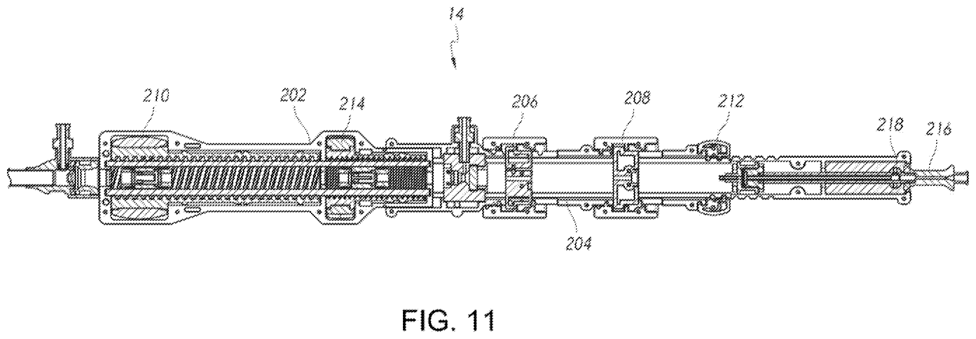

The handle 14 is located at the proximal end of the delivery system 10. An embodiment of a handle 14 is shown in FIG. 10. A cross-section of the handle 14 is shown in FIG. 11. The handle 14 can include a number of actuators, such as rotatable knobs, that can manipulate different components of the delivery system 10. The operation of the handle 14 is described with reference to delivery of a replacement valve prosthesis or implant 70, though the handle 14 and delivery system 10 can be used to deliver other devices as well.

The handle 14 is generally composed of two housings, a rail housing 202 and a delivery housing 204, the rail housing 202 being circumferentially disposed around the delivery housing 204. The inner surface of the rail housing 202 can include a screwable section configured to mate with an outer surface of the delivery housing 204. Thus, the delivery housing 204 is configured to slide (e.g., screw) within the rail housing 202, as detailed below. The rail housing 202 generally surrounds about one half the length of the delivery housing 204, and thus the delivery housing 204 extends both proximally and distally outside of the rail housing 202.

The rail housing 202 can contain two rotatable knobs, a distal pull wire knob 206 and a proximal pull wire knob 208. However, the number of rotatable knobs on the rail housing 202 can vary depending on the number of pull wires used. Rotation of the distal pull wire knob 206 can provide a proximal force, thereby providing axial tension on the distal pull wires 138 and causing the distal slotted section of the rail hypotube 136 to bend. The distal pull wire knob 206 can be rotated in either direction, allowing for bending in either direction, which can control anterior-posterior angles. Rotation of the proximal pull wire knob 208 can provide a proximal force, and thus axial tension, on the proximal pull wires 140, thereby causing the proximal slotted section 133 of the rail hypotube 136 to bend, which can control the medial-lateral angle. The proximal pull wire knob 208 can be rotated in either direction, allowing for bending in either direction. Thus, when both knobs are actuated, there can be two bends in the rail hypotube 136, thereby allowing for three-dimensional steering of the rail shaft 132, and thus the distal end of the delivery system 10. Further, the proximal end of the rail shaft 132 is connected on an internal surface of the rail housing 202.

The bending of the rail shaft 132 can be used to position the system, in particular the distal end, at the desired patient location, such as at the native tricuspid valve. In some embodiments, rotation of the pull wire knobs 206/208 can help steer the distal end of the delivery system 10 to a desired position proximal a valve to be treated, for example a tricuspid or mitral valve.

Moving to the delivery housing 204, the proximal ends of the inner shaft assembly 18, outer sheath assembly 22, mid shaft assembly 21, and nose cone shaft assembly 31 can be connected to an inner surface of the delivery housing 204 of the handle 14. Thus, they can move axially relative to the rail assembly 20 and rail housing 202.

A rotatable outer sheath knob 210 can be located on the distal end of the delivery housing 204, being distal to the rail housing 202. Rotation of the outer sheath knob 210 will pull the outer sheath assembly 22 in an axial direction proximally, thus pulling the capsule 106 away from the implant 70 and releasing the distal end 303 of implant 70. Thus the outer sheath assembly 22 is individually translated with respect to the other shafts in the delivery system 10. The distal end 303 of the implant 70 can be released first, while the proximal end 301 of the implant 70 can remain radially compressed between the inner retention member 40 and the outer retention member 42.

A rotatable mid shaft knob 214 can be located on the delivery housing 204, in some embodiments proximal to the rotatable outer sheath knob 210, being distal to the rail housing 202. Rotation of the mid shaft knob 212 will pull the mid shaft assembly 21 in an axial direction proximally, thus pulling the outer retention ring 42 away from the implant 70 and uncovering the inner retention member 40 and the proximal end 301 of the implant 70, thereby releasing the implant 70. Thus, the mid shaft assembly 21 is individually translated with respect to the other shafts in the delivery system 10.

Located on the proximal end of the delivery housing 204, and thus proximal to the rail housing 202, can be a rotatable depth knob 212. As the depth knob 212 is rotated, the entirety of the delivery housing 204 moves distally or proximally with respect to the rail housing 202 which will remain in the same location. Thus, at the distal end of the delivery system 10, the inner shaft assembly 18, outer sheath assembly 22, mid shaft assembly 21, and nose cone shaft assembly 31 together (e.g., simultaneously) move proximally or distally with respect to the rail assembly 20 while the implant 70 remains in the compressed configuration. In some embodiments, actuation of the depth knob 212 can sequentially move the inner shaft assembly 18, outer sheath assembly 22, mid shaft assembly 21, and nose cone shaft assembly 31 relative to the rail assembly 20. In some embodiments, actuation of the depth knob 212 can together move the inner shaft assembly 18, outer sheath assembly 22, and mid shaft assembly 21 relative to the rail assembly 20. Accordingly, the rail shaft 132 can be aligned at a particular direction, and the other assemblies can move distally or proximally with respect to the rail shaft 132 for final positioning while not releasing the implant 70. The components can be advanced approximately 1, 2, 3, 5, 6, 7, 8, 9, or 10 cm along the rail shaft 132. The components can be advanced more than approximately 1, 2, 3, 5, 6, 7, 8, 9, or 10 cm along the rail shaft 132. An example of this is shown in FIG. 2C. The capsule 106 and outer retention ring 42 can then be individually withdrawn with respect to the inner assembly 18 as discussed above, in some embodiments sequentially, releasing the implant 70. The assemblies other than the rail assembly 20 can then be withdrawn back over the rail shaft 132 by rotating the depth knob 212 in the opposite direction.

The handle 14 can further include a mechanism (knob, button, handle) 216 for moving the nose cone shaft 27, and thus the nose cone 28. For example, a knob 216 can be a portion of the nose cone assembly 31 that extends from a proximal end of the handle 14. Thus, a user can pull or push on the knob 216 to translate the nose cone shaft 27 distally or proximally individually with respect to the other shafts. This can be advantageous for proximally translating the nose cone 28 into the outer sheath assembly 22/capsule 106, thus facilitating withdraw of the delivery system 10 from the patient.

In some embodiments, the handle 14 can provide a lock 218, such as a spring lock, for preventing translation of the nose cone shaft 27 by the knob 216 discussed above. In some embodiments, the lock 218 can be always active, and thus the nose cone shaft 27 will not move without a user disengaging the lock 218. The lock can be, for example, a spring lock that is always engaged until a button 218 on the handle 14 is pressed, thereby releasing the spring lock and allowing the nose cone shaft 27 to translate proximally/distally. In some embodiments, the spring lock 218 allows one-way motion, either proximal or distal motion, of the nose cone shaft 27 but prevents motion in the opposite direction.

The handle 14 can further include a communicative flush port for flushing out different lumens of the delivery system 10. In some embodiments, a single flush port on the handle 14 can provide fluid connection to multiple assemblies. In some embodiments, the flush port can provide fluid connection to the outer sheath assembly 22. In some embodiments, the flush port can provide fluid connection to the outer sheath assembly 22 and the mid shaft assembly 21. In some embodiments, the flush port can provide fluid connection to the outer sheath assembly 22, the mid shaft assembly 21, and the rail assembly 20. In some embodiments, the flush port can provide fluid connection to the outer sheath assembly 22, the mid shaft assembly 21, the rail assembly 20, and the inner assembly 18. Thus, in some embodiments, the rail shaft 132, the outer retention ring 42, and the capsule 106 can all be flushed by a single flush port.

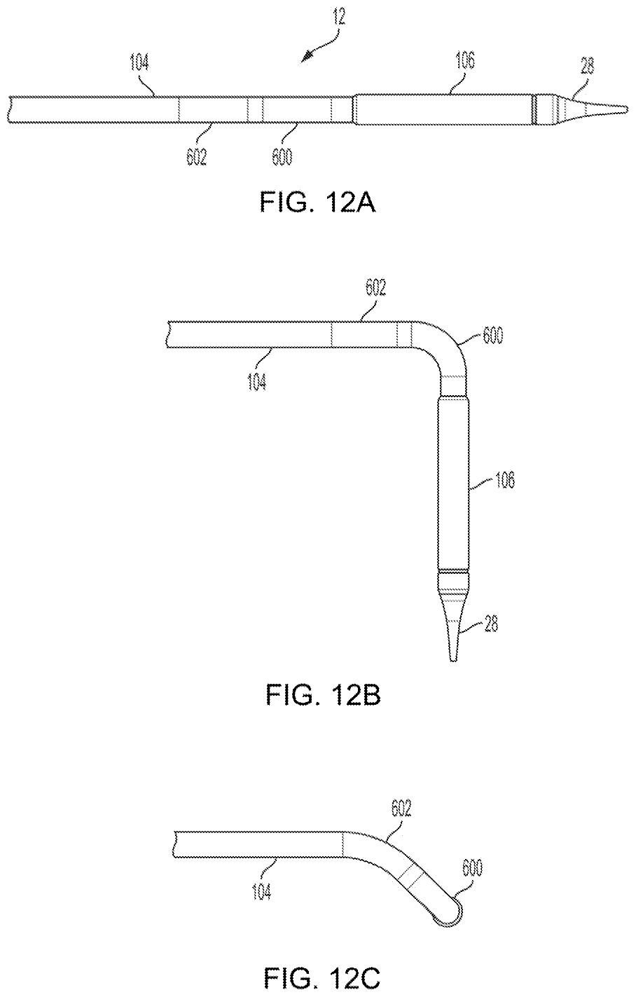

FIG. 12A illustrates a side view of a distal portion of the elongate shaft 12 with the elongate shaft 12 in a straightened configuration. The capsule 106 is shown positioned between the outer hypotube 104 and the nose cone 28.

The elongate shaft 12 may include one or more bend portions, which may allow the elongate shaft 12 to bend at the bend portions. In the embodiment shown in FIG. 12A, for example, the elongate shaft 12 includes two bend portions 600, 602. The bend portion 600 may correspond to the distal rail portion 601 shown in FIGS. 6B and 6C, and the bend portion 602 may correspond to the proximal rail portion 603 shown in FIGS. 6B and 6C. As such, the bend portions 600, 602 may be configured to bend the elongate shaft 12 in planes that are perpendicular from each other, with the bend portion 600 able to bend in what may be called a vertical plane, and the bend portion 602 able to bend in what may accordingly be called a horizontal plane. The bend portions 600, 602 may be configured to bend to orient the capsule 106 in the desired position for deployment of the implant 70 contained therein.

The capsule 106 (and the implant retention area 16 contained therein) may be configured to slide relative to the bend portions 600, 602 in the manners disclosed herein. For example, the outer sheath assembly 22, mid shaft assembly 21, inner shaft assembly 18, and nose cone assembly 31 may be configured to slide relative to the bend portions 600, 602 (as part of the rail assembly 20) to vary a distance or depth of the capsule 106 from the rail assembly 20. The outer sheath assembly 22 may be configured to slide relative to the rail assembly 20 to vary a distance of the implant retention area from the patient's tricuspid valve.

Referring to FIG. 12B, the bend portion 600, which is positioned proximal of the capsule 106, and is positioned between the capsule 106 and the bend portion 602, is shown to deflect the distal end of the elongate shaft 12 to a direction (which may be referred to as a downward direction as shown in FIG. 12B). The bend portion 600 deflects the distal end of the elongate shaft 12 in a plane (which may be referred to as a vertical plane). The bend portion 600 accordingly has varied the orientation of the capsule 106, the distal end of the elongate shaft 12, and the implant retention area 16 positioned within the capsule 106.

FIG. 12C illustrates a top view of the elongate shaft 12 shown in FIGS. 12A and 12B, with the bend portion 602 bent. In FIG. 12C, the bend portion 602, which is positioned proximal the bend portion 600 is shown to deflect the distal end of the elongate shaft 12 to a direction (which may be referred to as a rightward direction as shown in FIG. 12C). The bend portion 602 deflects the distal end of the elongate shaft 12 in a plane (which may be referred to as a horizontal plane). The bend portion 600 accordingly has varied the orientation of the capsule 106, the distal end of the elongate shaft 12, and the implant retention area 16 positioned within the capsule 106.

The bend portion 602 accordingly may deflect the bend portion 600 and the capsule 106 in a plane that is perpendicular to the plane that the bend portion 600 may deflect the capsule 106. The orthogonal planes of deflection may allow for three-dimensional steering of the capsule 106.

The bend portion 602 as shown in FIG. 12C may be configured to deflect the distal end of the elongate shaft 12 to a rightward direction. Such direction of deflection may be provided by the configuration of pull wires shown in FIG. 6C.

Additional or varied movement of the elongate shaft 12 may be desired. Such additional or varied movement may be desired for a variety of reasons, which may include a variety of patient anatomies to be navigated with the distal end of the elongate shaft 12 or varied uses of the elongate shaft 12.

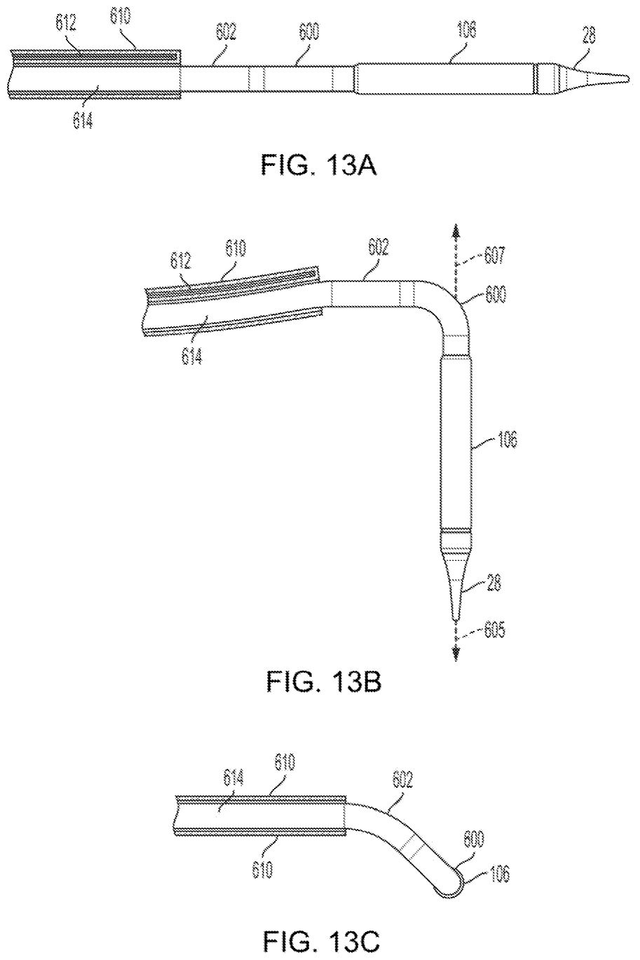

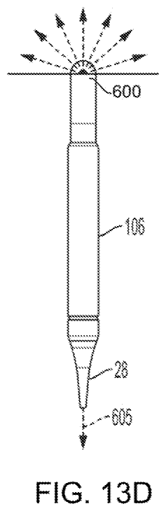

FIGS. 13A—D illustrate an embodiment in which a deflection mechanism may be utilized to provide deflection of a portion of the elongate shaft 12. Referring to FIG. 13A, the deflection mechanism may include a sheath 610 that extends over a portion 614 of the elongate shaft 12. The portion 614 of the elongate shaft 12 may comprise a portion that is positioned proximal of the bend portion 602 and the bend portion 600. However, in other embodiments the sheath 610 may extend over other portions of the elongate shaft 12, possibly extending to the distal end of the elongate shaft 12.

The sheath 610 is shown in cross section in FIG. 13A and may be configured to deflect to provide the deflection of the elongate shaft 12. The sheath may include a control device that is utilized to control deflection of the sheath 610. The control device may comprise a pull tether 612 as shown in FIG. 13A, which may comprise a pull wire or other forms of tethers. In other embodiments, other forms of control devices may be utilized such as gears, rails, or other forms of control devices. The pull tether 612 may be oriented on the elongate shaft 12 such that retraction of the pull tether 612 may deflect the elongate shaft 12 in a direction towards the pull tether 612.

Referring to FIG. 13B, the bend portion 600 has deflected the distal end of the elongate shaft 12 to a direction 605 (which may be referred to as a downward direction as shown in FIG. 13B). The deflection mechanism, however, has deflected the portion 614 of the elongate shaft 12 that is positioned proximal of the bend portion 600 and bend portion 602 to deflect the bend portions 600, 602 towards a direction 607 that is opposed from the direction 605 that the bend portion 600 has deflected the distal end of the elongate shaft 12. The deflection mechanism has also deflected the bend portion 602, bend portion 600, capsule 106, implant retention area 16 contained within the capsule 106, and the nose cone 28 towards the direction that is opposed from the direction that the bend portion 600 has deflected the distal end of the elongate shaft 12. The deflection mechanism accordingly may be utilized to deflect the elongate shaft 12 in order to create height or distance from the distal end of the elongate shaft 12 to a desired implantation location.

The deflection mechanism has deflected a portion 614 of the elongate shaft 12 in the same plane (coplanar) that the bend portion 600 has deflected the distal end of the elongate shaft 12.

The deflection mechanism may be utilized to allow the bend portions 600, 602 to bend the respective distal portions of the elongate shaft 12, in a similar manner as shown in FIGS. 12A-C. Referring to FIG. 13C, for example, the deflection mechanism is deflecting the proximal portion 614 of the elongate shaft 12, however, the bend portion 600 continues to deflect the distal end of the elongate shaft 12 in the direction shown in FIG. 13B, and the bend portion 602 deflects the bend portion 600 in a perpendicular direction as described in regard to FIG. 12C. The deflection mechanism in the form of the elongate sheath 610 continues to deflect a portion 614 of the elongate shaft 12 that is proximal the bend portion 600 and bend portion 602 to deflect the bend portions 600, 602 towards a direction that is away from the direction that the bend portion 600 has deflected the distal end of the elongate shaft 12.

The deflection mechanism may be configured to provide multiple directions of deflection of the portion 614 of the elongate shaft 12 that is proximal the bend portion 600 and bend portion 602. The deflection mechanism, in the form of the sheath 610, for example, may be configured to rotate about the portion of the elongate shaft 12 that the sheath 610 extends over. Such rotation may move the position of the pull tether 612 relative to the elongate shaft 12 to cause the elongate shaft 12 to deflect towards the varied position of the pull tether 612. As such, a variety of directions of deflection of the elongate shaft 12 may result. FIG. 13D for example, illustrates a front view of the elongate sheath showing multiple directions (via the arrows) that are opposed to the direction 605 that the bend portion 600 may be deflected towards.

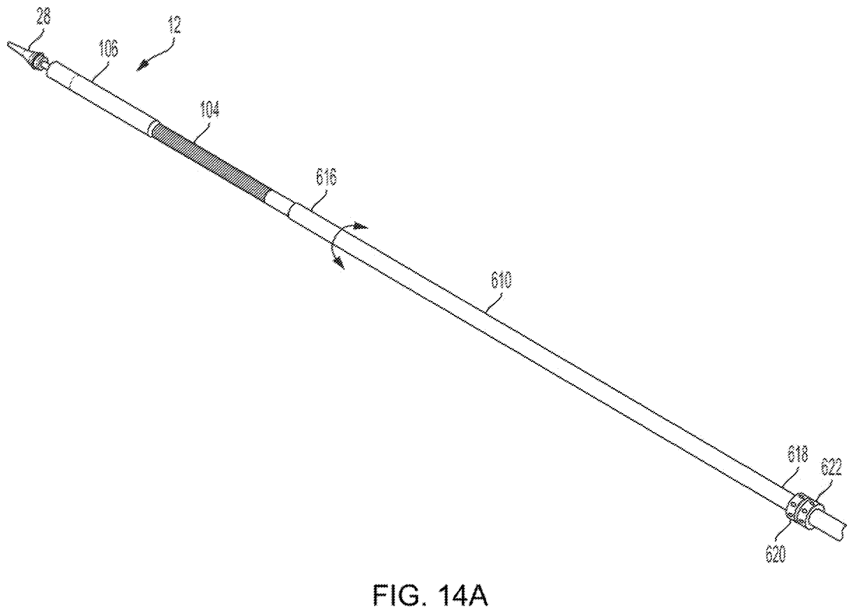

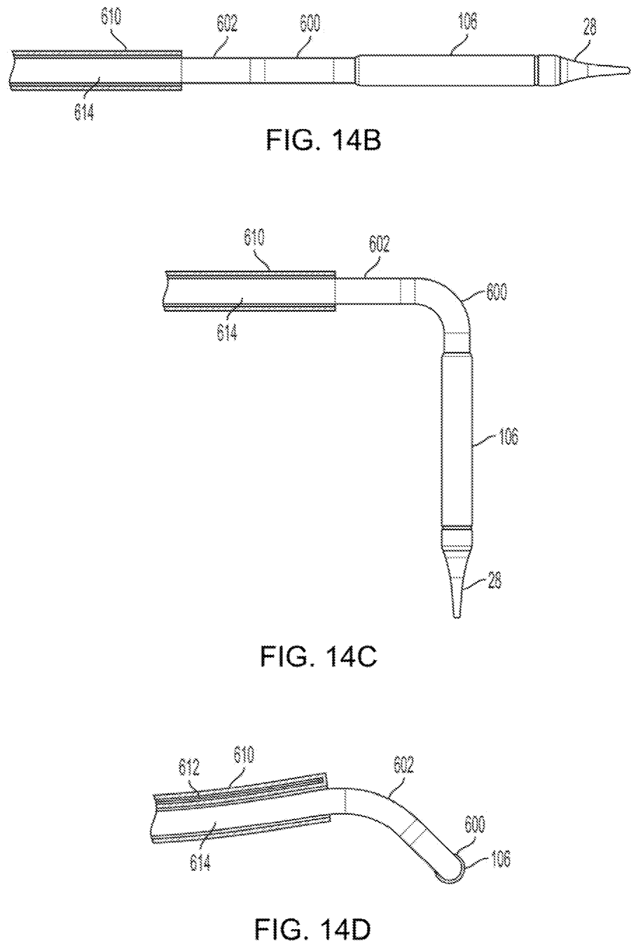

FIG. 14A illustrates a perspective view of the sheath 610 extending over the elongate shaft 12. The sheath 610 may be utilized in lieu of, or in combination with the sheath 51 shown in FIG. 1. The sheath 610 may have a distal end 616 and a proximal end 618. The proximal end 618 of the sheath 610 may be coupled to a rotation control housing 620 that may be utilized to control rotation of the sheath 610 about the elongate shaft 12. A user, such as a surgeon or another user, may grasp the rotation control housing 620 to control rotation of the sheath 610 about the elongate shaft 12, to thereby control the direction of deflection of the elongate shaft 12 caused by the sheath 610. The proximal end 618 of the sheath 610 may alternatively or additionally couple to a deflection control housing 622, which may be utilized to draw the pull tether 612 proximally to deflect the sheath 610, and may be utilized to release the pull tether 612 in a distal direction to straighten the sheath 610. The deflection control housing 622 may be configured for a user, such as a surgeon or another user, to grasp, to control deflection of the sheath 610.

The control housings 620, 622 may be integrated to form a single control housing as desired. In one embodiment, the controls of the control housings 620, 622 may be integrated in the handle 14, or may remain separate from the handle 14 as desired.

FIGS. 14B-D illustrate the deflection mechanism in the form of the sheath 610 rotated 90° about the elongate shaft 12 relative to the position shown in FIG. 13A. The sheath 610 may be rotated through use of the rotation control housing 620, or through another method as desired. The relative position of the pull tether 612 has rotated 90° as shown in FIG. 14D. Referring to FIG. 14D, the sheath 610 may deflect the elongate shaft 12 towards a direction that is perpendicular to the direction that the bend portion 600 has deflected the distal end of the elongate shaft 12. The sheath 610 may deflect the elongate shaft 12 in the same plane that the bend portion 602 deflects a portion of the elongate shaft 12 that is distal to the bend portion 602.

The deflection mechanism in the form of the sheath 610 may have a variety of orientations relative to the elongate shaft 12, at any angular position relative to the elongate shaft 12 as desired. As such, the deflection mechanism in the form of the sheath 610 may be configured to deflect the portion 614 of the elongate shaft 12 in multiple directions, which may or may not be perpendicular to the direction that the bend portion 600 has deflected the distal end of the elongate shaft 12. The deflection mechanism in the form of the sheath 610 may deflect the portion 614 of the elongate shaft 12 towards a variety of directions that are opposed to the direction that the bend portion 600 has deflected the distal end of the elongate shaft 12, which may include a direction that is directly opposite the direction that the bend portion 600 has deflected the distal end of the elongate shaft 12 (at 180° degrees) and a variety of other directions that are in between direct opposition (at 180° degrees) and a perpendicular direction (at 90°) (e.g., 135°, among others).

The deflection mechanism in the form of the sheath 610 may be configured to deflect the portion 614 of the elongate shaft 12 in a direction that is towards the direction that the bend portion 600 has deflected the distal end of the elongate shaft 12, if the sheath 610 is rotated to provide such deflection.

The deflection mechanism in the form of the sheath 610 may be configured to vary the direction of deflection of the portion 614 not only via rotation of the sheath 610 but in embodiments may be configured with multiple pull tethers or other control devices that allow for varied directions of deflection of the sheath 610 without rotation of the sheath 610. For example, if four equally spaced pull tethers (spaced 90° from each other) are utilized with the sheath 610, then a combination of movement of the pull tethers may provide a variety of directions of deflection of the sheath 610. Other configurations may be utilized to vary the direction of deflection of the sheath 610. At least one pull tether may be utilized in embodiments.

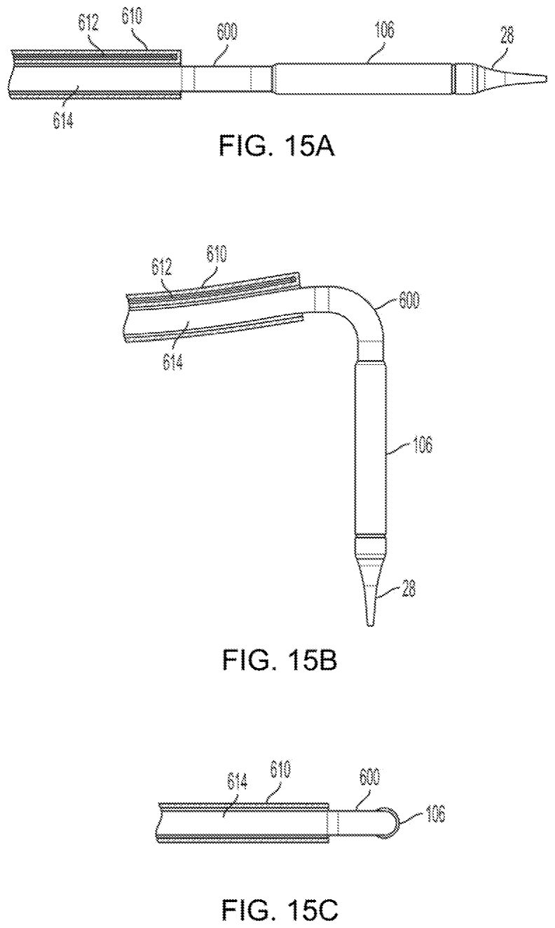

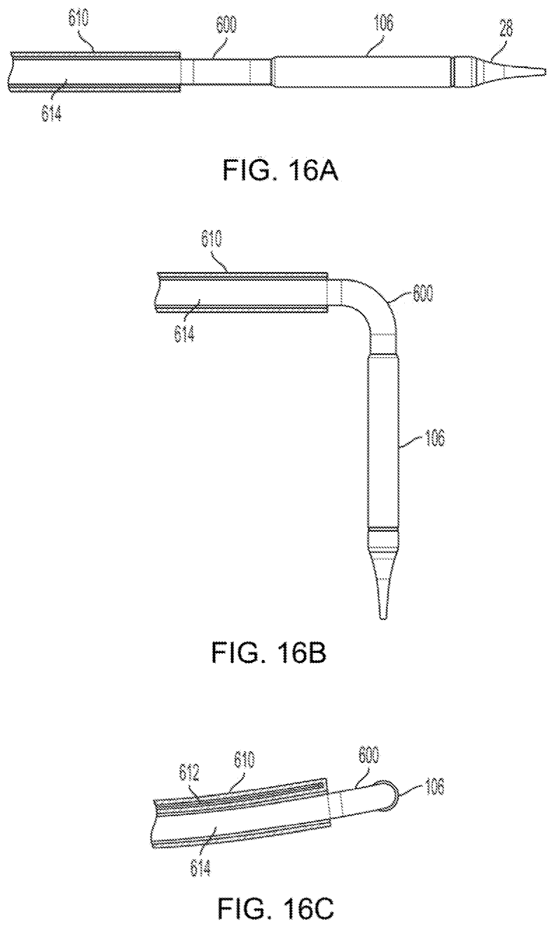

The embodiments of FIGS. 13A-14D illustrate an elongate shaft 12 having two bend portions 600, 602 configured to bend in perpendicular planes. However, the configuration and use of the bend portions 600, 602 may be varied in other embodiments as desired. For example, FIGS. 15A-16C illustrate an embodiment in the bend portion 602 has been excluded, and where the sheath 610 controls deflection of the elongate shaft 12 in lieu of the bend portion 602. The sheath 610 accordingly may be configured to deflect the elongate shaft 12 towards a direction that is opposed to the direction that the bend portion 600 has deflected the distal end of the elongate shaft 12, which may include a direction that is directly opposite the direction that the bend portion 600 has deflected the distal end of the elongate shaft 12 (at 180° degrees) and a variety of other directions that are in between direct opposition (at 180° degrees) and a perpendicular direction (at) 90° (e.g., 135°, among others). FIGS. 15A—C illustrate the sheath 610 deflecting the portion of the elongate shaft 12 to deflect the bend portion 600 towards a direction that is opposed to the direction that the bend portion 600 has deflected the distal end of the elongate shaft 12.

The sheath 610 may be rotated from the orientation shown in FIGS. 15A—C, to vary the direction of deflection of the portion 614. FIGS. 16A—C illustrate the sheath 610 rotated 90° from the orientation shown in FIGS. 15A—C, to deflect the portion 614 in a plane that is perpendicular to the plane of deflection of the bend portion 600. As discussed in regard to FIGS. 13A-14D, the sheath 610 may in other embodiments be configured with multiple pull tethers or other control devices that allow for varied directions of deflection of the sheath 610 without rotation of the sheath 610.

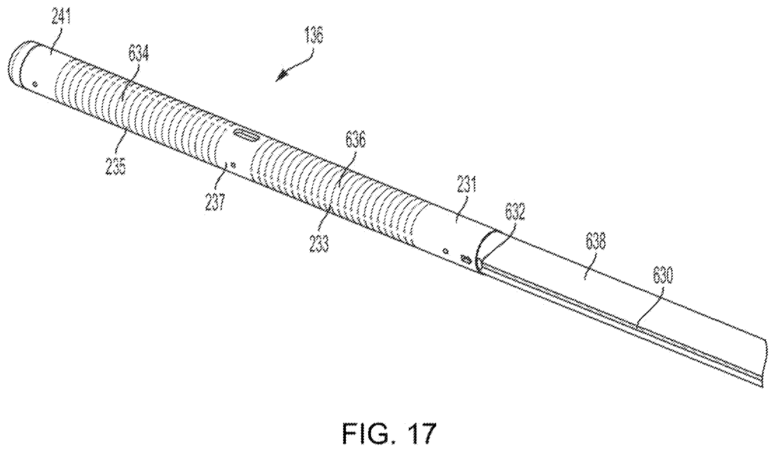

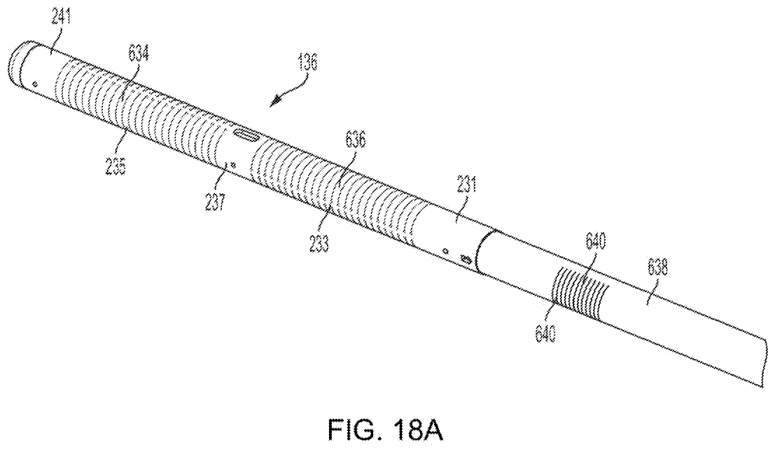

Other forms of deflection mechanisms may be utilized. For example, FIG. 17 illustrates an embodiment of a deflection mechanism in the form of a pull tether 630. The pull tether 630 may have a distal end 632 that is coupled to a portion of the elongate shaft 12, for example, the rail shaft 132. The rail shaft 132 may extend over an inner shaft as disclosed herein, and may have an outer sheath extending over the rail shaft 132 as disclosed herein. The distal end 632 may couple to the rail proximal shaft 134 or another portion of the rail shaft 132 that is proximal the rail hypotube 136 or the bend portions 634, 636 of the rail shaft 132. For example, as shown in FIG. 17, the distal end 632 may couple to a portion that is proximal the uncut (or unslotted) hypotube section 231.

The pull tether 630 may be configured to be retracted to deflect the portion 638 of the rail shaft 132, and thus the elongate shaft 12, that is proximal the bend portions 634, 636. As such, the bend portion 634 may be configured to deflect the distal end of the elongate shaft 12 in a direction, and the pull tether 630 may be configured to deflect the elongate shaft 12 to deflect the bend portions 634, 636 towards a direction that is opposed to the direction that the bend portion 634 has deflected the distal end of the elongate shaft 12. The pull tether 630 may be coupled to the rail shaft 132 at a position and with an orientation that opposes the direction that the bend portion 634 has deflected the distal end of the elongate shaft 12 when the pull tether 630 is retracted.

A single pull tether 630 is shown in FIG. 17, however multiple pull tethers may be utilized in other embodiments as desired. For example, if four equally spaced pull tethers (spaced 90° from each other) are coupled to the rail shaft 132, then a combination of movement of the pull tethers may provide a variety of directions of deflection of the elongate shaft 12. Other configurations may be utilized to vary the direction of deflection of the elongate shaft 12. The one or more pull tethers accordingly may be configured to deflect the elongate shaft 12 towards a direction that is opposed to the direction that the bend portion 634 has deflected the distal end of the elongate shaft 12, which may include a direction that is directly opposite the direction that the bend portion 634 has deflected the distal end of the elongate shaft 12 (at 180° degrees) and a variety of other directions that are in between and include direct opposition (at 180° degrees) and a perpendicular direction (at 90°) (e.g., 135°, among others).

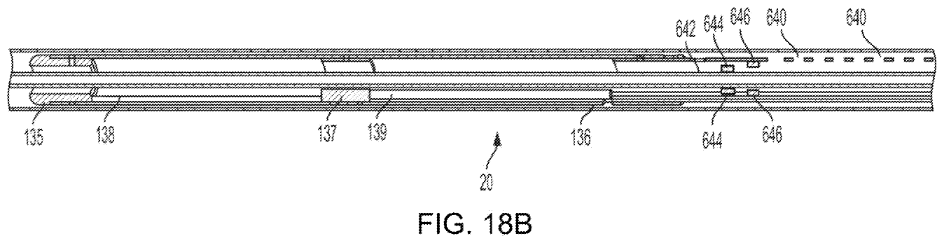

FIGS. 18A—B illustrate an embodiment of a deflection mechanism including cuts 640 in a portion of the elongate shaft 12 and a pull shaft 642 that may be retracted to cause the elongate shaft 12 to deflect at the location of the cuts 640. Referring to FIG. 18A, the cuts 640 may be positioned on the rail shaft 132 at a desired location. Such a location may be proximal the rail hypotube 136 or the bend portions 634, 636 of the rail shaft 132. For example, as shown in FIG. 18A, the cuts 640 may be proximal the uncut (or unslotted) hypotube section 231.

The cuts 640 may have a configuration that biases the rail shaft 132 to deflect at the cuts 640 and in a direction that is away from the direction that the bend portion 634 has deflected the distal end of the elongate shaft 12.

Referring to FIG. 18B, a cross sectional view of the rail shaft 132 is shown. The deflection mechanism may include the inner shaft or pull shaft 642, which may be positioned within the rail shaft 132. The pull shaft 642 may be positioned between the rail shaft 132 and an inner shaft such as the inner shaft assembly 18 or the nose cone assembly 31. In other embodiments, the inner shaft or pull shaft 642 may be provided in other locations.

The inner shaft or pull shaft 642 may include a stopper 644 coupled thereto. The rail shaft 132, and particularly the portion of the rail shaft 132 distal the cuts 640 may include a stopper 646. The deflection mechanism may be configured that as the pull shaft 642 is drawn proximally, the stopper 644 contacts the stopper 646 and applies a proximal force to the rail shaft 132 and particularly the portion of the rail shaft 132 including the cuts 640. The cuts 640, providing a biased direction of deflection, may cause the rail shaft 132 and accordingly the elongate shaft 12 to deflect in this direction of deflection, which is in a direction that is opposed to the direction that the bend portion 634 has deflected the distal end of the elongate shaft 12. The pull shaft 642 may then be moved distally to reduce the force between the stoppers 644, 646 to cause the rail shaft 132 to straighten. FIG. 18B shows the stoppers 644, 646 separate from each other, however, the inner shaft or pull shaft 642 may be drawn proximally for the stoppers 644, 646 to contact each other.

A single pull shaft 642 is shown in FIG. 18B, however multiple pull shafts may be utilized in other embodiments as desired. For example, if four equally spaced pull shafts (spaced 90° from each other) with corresponding stoppers are utilized, then a combination of movement of the pull shafts may provide a variety of directions of deflection of the elongate shaft 12. The cut pattern may be provided such that a variety of directions of deflection are possible. Other configurations may be utilized to vary the direction of deflection of the elongate shaft 12. The one or more pull shafts accordingly may be configured to deflect the elongate shaft 12 to deflect the bend portions 634, 636 towards a direction that is opposed to the direction that the bend portion 634 has deflected the distal end of the elongate shaft 12, which may include a direction that is directly opposite the direction that the bend portion 634 has deflected the distal end of the elongate shaft 12 (at 180° degrees) and a variety of other directions that are in between and include direct opposition (at 180° degrees) and a perpendicular direction (at 90°) (e.g., 135°, among others).

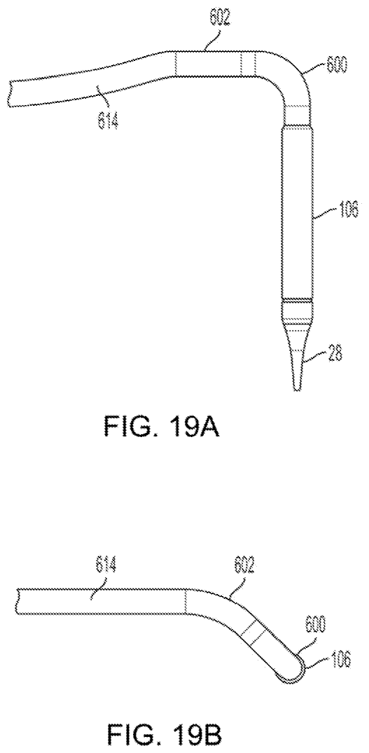

FIGS. 19A—B illustrate an external view of the embodiments of FIGS. 17-18B. The sheath 610 may or may not be utilized with the deflection mechanisms shown in FIGS. 17-18B. As such, the outer sheath assembly 22 may comprise the outer surface of the elongate shaft 12, with the deflection mechanisms contained within the outer sheath assembly 22.

As shown in FIG. 19A, the bend portion 600 may deflect the distal end of the elongate shaft 12 to a direction. The deflection mechanism may deflect the proximal portion 614 of the elongate shaft 12 to deflect the bend portion 600 towards a direction that is opposed to the direction of the distal end of the elongate shaft 12. FIG. 19B illustrates that the bend portions 600, 602 may continue to operate to deflect the respective distal portions of the elongate shaft 12.

The deflection mechanisms may be utilized to provide for additional or varied movement of the elongate shaft 12. Such additional or varied movement may be desired for a variety of reasons, which may include a variety of patient anatomies to be navigated with the distal end of the elongate shaft 12 or varied uses of the elongate shaft 12.

The deflection mechanisms may be utilized to move the elongate shaft 12 for delivery of a replacement heart valve, which may include a replacement tricuspid valve. Although many of the embodiments herein are discussed in regard to a replacement tricuspid valve, the deflection mechanisms may be utilized for a variety of other implementations including delivery of mitral replacement valves, or aortic or pulmonary valves, or for valve repair procedures, including tricuspid or mitral valve repair or aortic or pulmonary valve repair.

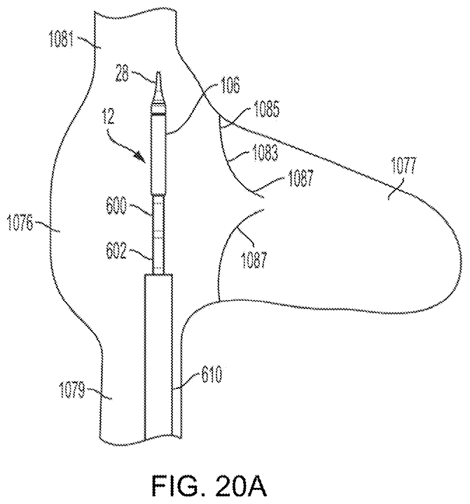

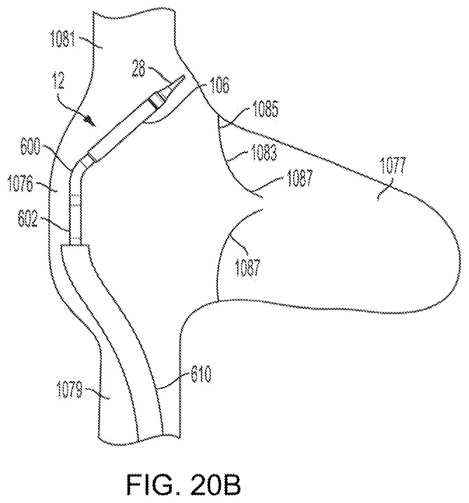

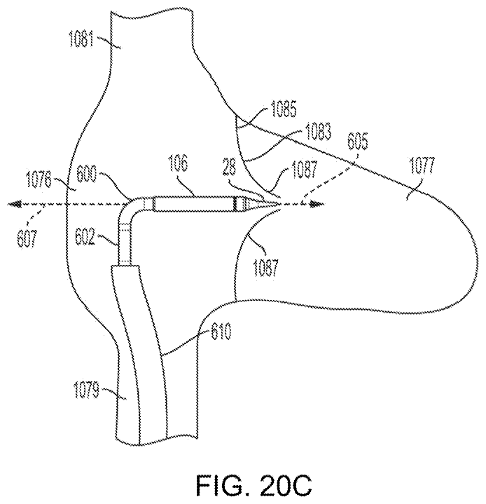

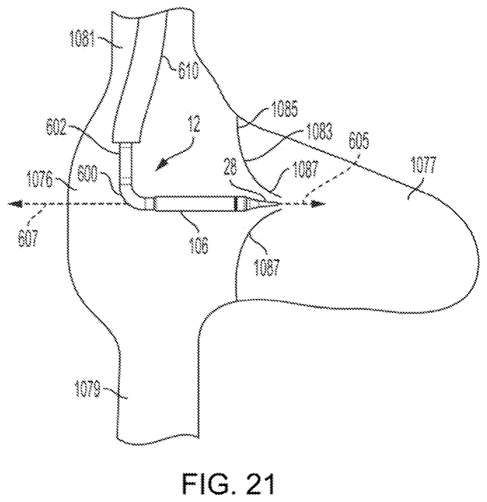

FIGS. 20A-21 illustrate a use of the elongate shaft 12 to treat a patient's tricuspid valve. The elongate shaft 12 may be passed into the patient's body in an endovascular manner, which may include percutaneous entry of the patient's vasculature. For example, the elongate shaft 12 may be entered into the ipsilateral femoral vein and advanced toward the right atrium 1076. Other entry methods may be utilized in other embodiments, including a transjugular approach, or other approaches including transapical approaches.

As shown in FIG. 20A, the elongate shaft 12 may be advanced through the inferior vena cava 1079 to approach or reach the right atrium 1076 of the patient's heart. The right ventricle 1077, the tricuspid valve 1083 including tricuspid valve leaflets 1087, the tricuspid valve annulus 1085, and the superior vena cava 1081 are also shown.

The delivery system may include use of the deflection mechanisms discussed herein. As shown in FIG. 20A, the deflection mechanism in the form of the sheath 610 may be utilized, however it is understood that other forms of deflection mechanisms may be utilized, including the deflection mechanisms shown in FIGS. 17-19B.

The elongate shaft 12 may be advanced towards the right atrium 1076, with the distal end of the elongate shaft 12 to be deflected such that the capsule 106 and thus the implant retention area 16 are oriented to deploy the implant contained therein to the tricuspid valve 1083 in the desired manner. As represented in FIG. 20A, the distal end of the elongate shaft 12 may require deflection to a direction towards the tricuspid valve 1083, to align the distal end of the elongate shaft 12 and the capsule 106 (and the deployment port at the distal end of the capsule for the implant to be deployed from) with the central axis of the tricuspid valve 1083. For other methods of deployment, other directions of deflection may be desired.

The bend portions 600, 602 may be utilized to deflect the distal end of the elongate shaft 12 to the desired direction. The bend portions 600, 602 may be configured to deflect the distal end of the elongate shaft in perpendicular planes, to provide two planes of deflection. The bend portions 600, 602 may be configured similarly as shown in FIG. 6C, with the proximal bend portion 602 configured to deflect the distal portions of the elongate shaft 12 in a rightward (or anterior) direction relative to a downward (or ventricular) direction of deflection of the distal bend portion 600. Such a configuration may account for the position of the tricuspid valve 1083 relative to the inferior vena cava 1079 within a human heart.

Additional movement, however, may be provided by the deflection mechanisms disclosed herein. The deflection mechanism in the form of the sheath 610 may be utilized to deflect a proximal portion of the elongate shaft 12 to deflect the bend portions 600, 602 in a direction opposed to the direction that the bend portion 600 has deflected the distal end of the elongate shaft 12. Such a deflection may include deflecting the proximal portion of the elongate shaft 12 and the bend portions 600, 602 in an atrial direction (or providing a height from the tricuspid valve 1083). The capsule 106 and distal end of the elongate shaft 12 may also be deflected in an atrial direction (or providing a height from the tricuspid valve 1083).

The deflection mechanism may be utilized to account for a geometry of the patient's anatomy, which may include the geometry of the right atrium 1076, the size and relative position of the tricuspid valve 1083, and the geometry of the inferior vena cava 1079. For example, as shown in FIG. 20A, the distance of the bend portion 600 to the distal end of the elongate shaft 12 may be such that the bending radius of the elongate shaft 12 distal the bend portion 600 is too large to properly direct the distal end of the elongate shaft 12 to the tricuspid valve 1083, depending on the geometry of the patient's right atrium 1076. The deflection mechanism accordingly may be utilized to deflect the bend portion 600 towards a direction opposed to the direction that the bend portion 600 deflects the distal end of the elongate shaft 12.

Referring to FIG. 20B, the deflection mechanism in the form of the sheath 610 may deflect the proximal portion of the elongate shaft 12, as discussed herein. The deflection of the proximal portion of the elongate shaft 12 may occur wholly or partially (at least partially) within the patient's inferior vena cava 1079. The deflection may move the bend portions 600, 602 to create height from the tricuspid valve 1083 in a direction away from the tricuspid valve. As such, the distal end of the elongate shaft 12 may have greater clearance space for the bend portion 600 to deflect the distal end of the elongate shaft 12 towards the tricuspid valve 1083. As shown in FIG. 20B, the deflection mechanism may form a curve of the proximal portion of the sheath, although other forms of deflection may result. The bend portion 600 has begun deflection of the distal end of the elongate shaft 12 in FIG. 20B.

Referring to FIG. 20C, the bend portion 600 has deflected the distal end of the elongate shaft 12 to a direction 605. The direction may be aligned with the axis of the tricuspid valve 1083 or otherwise may be directed in a desired orientation. The deflection mechanism in the form of the sheath 610 has deflected the proximal portion of the elongate shaft 12 to deflect the bend portion 600 in a direction 607 that is opposed to the direction 605. As such, the capsule 106 has increased height from the tricuspid valve 1083, to allow for deployment of the implant contained therein.

The deflection mechanism in the form of the sheath 610 may provide various directions of deflection of the proximal portion of the elongate shaft 12, and correspondingly various directions of deflection of the bend portions 600, 602, the capsule 106, and the distal end of the elongate shaft 12. As discussed in regard to FIGS. 14A—D, for example, the sheath 610 may provide for various directions of deflection, including perpendicular to the direction of deflection provided by the bend portion 600, and towards the direction of deflection provided by the bend portion 600. Such various directions of deflection may allow for additional maneuverability and variation of trajectory of the distal end of the elongate shaft 12 within the right atrium, and within the inferior vena cava 1079 or other area that the elongate shaft 12 is positioned within. The deflection mechanism in the form of the sheath 610 may provide for deflection in both the atrial and ventricular directions, and for various other directions.

The operation of the deflection mechanism shown in FIGS. 20A—C is not limited to the sheath 610 shown in FIGS. 13A-14D, but includes use of the deflection mechanisms shown in FIGS. 15A-19B as well. For example, the proximal bend portion 602 may be excluded, with the sheath 610 providing deflection of this portion of the elongate shaft 12 as discussed in regard to FIGS. 15A-16C. Further, the deflection mechanism may be positioned within the outer sheath assembly 22 as discussed in regard to the embodiments of FIGS. 17-19B. Various directions of deflection in both the atrial and ventricular directions, and various other directions may result.

The deflection mechanisms may be utilized to deflect the proximal portion of the elongate shaft 12 in one or more planes that are not perpendicular to the plane that the bend portion 600 deflects the distal end of the elongate shaft 12.

FIG. 21 illustrates use of the deflection mechanism in an approach from the superior vena cava 1081. The approach may be a transjugular approach, or via another entry point into the patient's body. The bend portions 600, 602 may be configured similarly as shown in FIG. 6B, with the proximal bend portion 602 configured to deflect the distal portions of the elongate shaft 12 in a leftward (or posterior) direction relative to a downward (or ventricular) direction of deflection of the distal bend portion 600. Such a configuration may account for the position of the tricuspid valve 1083 relative to the superior vena cava 1081 within a human heart. A method may include passing a delivery apparatus for an implant into a patient's right atrium.

The deflection mechanism, similarly as shown in FIGS. 20A—C, may deflect the proximal portion of the elongate shaft 12 to deflect the bend portion 600 in a direction 607 that is opposed to the direction 605 that the bend portion 600 has deflected the distal end of the elongate shaft 12. Similarly, as discussed in regard to FIGS. 13A-19B, other forms of deflection mechanisms may be utilized and other directions of deflection may result.

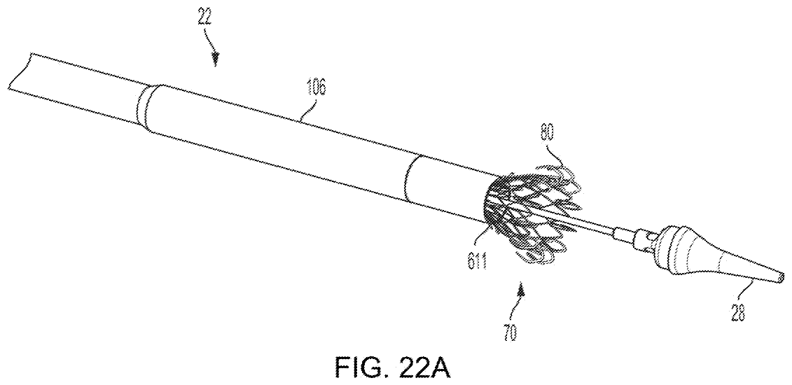

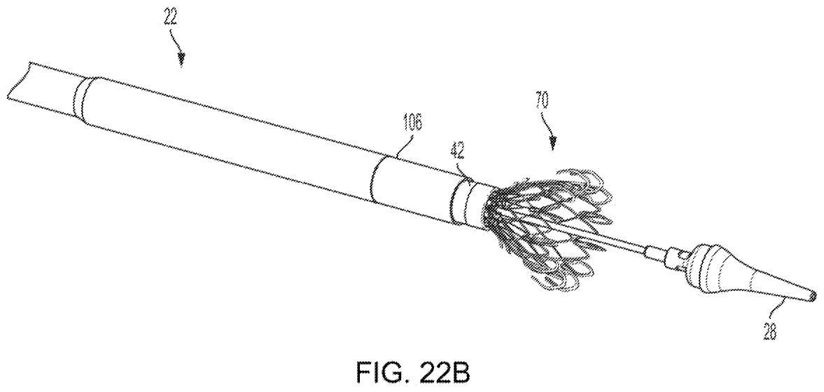

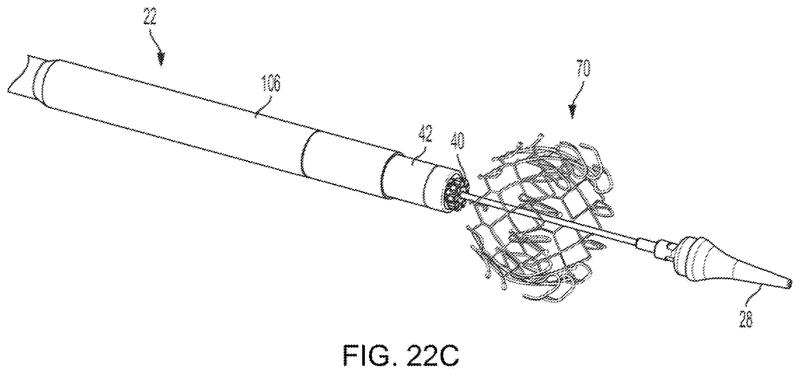

The implant 70 contained within the capsule 106 may be deployed to be positioned within the tricuspid valve annulus 1085, to replace the native tricuspid valve 1083. Upon the distal end of the elongate shaft 12 being oriented as desired relative to the native tricuspid valve 1083, a release mechanism may be utilized to deploy the implant 70 from the deployment port 611 at the distal end of the capsule 106. A height of the deployment port 611 relative to the valve may be varied by deflecting the delivery apparatus within an inferior vena cava or a superior vena cava. FIGS. 22A—C illustrate the release mechanism of the delivery system 10. During the initial insertion of the implant 70 and the delivery system 10 into the body, the implant 70 can be located within the system 10, similar to as shown in FIG. 2A. The distal end 303 of the implant 70, and specifically the distal anchors 80, are restrained within the capsule 106 of the outer sheath assembly 22, thus preventing expansion of the implant 70. Similar to what is shown in FIG. 2A, the distal anchors 80 can extend distally when positioned in the capsule. The proximal end 301 of the implant 70 is restrained within the capsule 106 and within a portion of the inner retention member 40 and thus is generally constrained between the capsule 106 and the inner retention member 40.

Once the implant 70 is loaded into the delivery system 10, a user can thread a guide wire into a patient to the desired location. The guide wire passes through the lumen of the nose cone assembly 31, and thus the delivery system 10 can be generally advanced through the patient's body following the guide wire. The delivery system 10 can be advanced by the user manually moving the handle 14 in an axial direction. In some embodiments, the delivery system 10 can be placed into a stand while operating the handle 14 controls.

Once generally in heart, the user can begin the steering operation of the rail assembly 20, and particularly the bend portions 600, 602 using the distal pull wire knob 206 and/or the proximal pull wire knob 208. By turning either of the knobs, the user can provide flexing/bending of the rail assembly 20 (either on the distal end or the proximal end), thus bending the distal end of the delivery system 10 in one, two, or more locations into the desired configuration. As discussed above, the user can provide multiple bends in the rail assembly 20 to direct the delivery system 10 towards the tricuspid valve. In particular, the bends of the rail assembly 20 can direct a distal end of the delivery system 10, and thus the capsule 106, along the center axis passing through the native tricuspid valve and towards the tricuspid valve. Thus, when the outer sheath assembly 22, mid shaft assembly 21, inner assembly 18, and nose cone assembly 31 are together advanced over the rail assembly 20 with the compressed implant 70, the capsule 106 proceed directly in line with the axis for proper release of the implant 70.

The user may utilize the deflection mechanisms as well, which may create height from the native tricuspid valve or may otherwise orient the distal end of the elongate shaft 12 as desired. The height of a bend portion of the elongate shaft 12 may be varied from the tricuspid valve.

The system 10 can be positioned to a particular location in a patient's body, such as at the native tricuspid valve, through the use of the bend portions and deflection mechanisms discussed herein or other techniques.

The user can also rotate and/or move the handle 14 itself in a stand for further fine tuning of the distal end of the delivery system 10. The user can continually turn the proximal and/or distal pull wire knobs 208/206, as well as moving the handle 14 itself, to orient the delivery system 10 for release of the implant 70 in the body. The user can also further move the other assemblies relative to the rail assembly 20, such as proximally or distally.

The user may utilize control mechanisms such as the rotation control housing 620 or deflection control housing 622 as shown in FIG. 14A or other control mechanisms to control operation of the deflection mechanism.

Upon the distal end of the elongate shaft 12 being oriented as desired, the user may rotate the depth knob 212. As discussed, rotation of this knob 212 together advances the inner shaft assembly 18, mid shaft assembly 21, outer sheath assembly 22, and nose cone assembly 31 over/through the rail assembly 20 while the implant 70 remains in the compressed configuration within the implant retention area 16. Due to the rigidity of, for example, either the inner shaft assembly 18, the mid shaft assembly 21, and/or the outer sheath assembly 22, these assemblies proceed straight forward in the direction aligned by the rail assembly 20.

Once in the release position, the user can rotate the outer sheath knob 210, which individually translates the outer sheath assembly 22 (and thus the capsule 106) with respect to the other assemblies, in particular the inner assembly 18, in a proximal direction towards the handle 14 as shown in FIG. 22A. By doing so, the distal end 303 of implant 70 is uncovered in the body, allowing for the beginning of expansion. At this point, the distal anchors 80 can flip proximally and the distal end 303 begins to expand radially outwardly. For example, if the system 10 has been delivered to a native tricuspid valve location, the distal anchors 80 expand radially outwardly within the right ventricle. The distal anchors 80 can be located above the papillary heads, but below the tricuspid valve annulus and tricuspid valve leaflets.

In some embodiments, the distal anchors 80 may contact and/or extend between the chordae in the right ventricle, as well as contact the leaflets, as they expand radially. In some embodiments, the distal anchors 80 may not contact and/or extend between the chordae or contact the leaflets. Depending on the position of the implant 70, the distal ends of the distal anchors 80 may be at or below where the chordae connect to the free edge of the native leaflets.

As shown in the illustrated embodiment, the distal end 303 of the implant 70 is expanded outwardly. It should be noted that the proximal end 301 of the implant 70 can remain covered by the outer retention ring during this step such that the proximal end 301 remains in a radially compacted state. At this time, the system 10 may be withdrawn proximally so that the distal anchors 80 capture and engage the leaflets of the tricuspid valve, or may be moved proximally to reposition the implant 70. For example, the assemblies may be proximally moved relative to the rail assembly 20. Further, the deflection mechanisms may be utilized to draw the elongate shaft 12 proximally relative to the tricuspid valve. Further, the system 10 may be torqued, which may cause the distal anchors 80 to put tension on the chordae through which at least some of the distal anchors may extend between. However, in some embodiments the distal anchors 80 may not put tension on the chordae. In some embodiments, the distal anchors 80 may capture the native leaflet and be between the chordae without any further movement of the system 10 after withdrawing the outer sheath assembly 22.

During this step, the system 10 may be moved proximally or distally to cause the distal or ventricular anchors 80 to properly capture the native tricuspid valve leaflets. This can be done by moving the outer sheath assembly 22, mid shaft assembly 21, inner assembly 18, and nose cone assembly 31 with respect to the rail assembly 20. In particular, the tips of the ventricular anchors 80 may be moved proximally to engage a ventricular side of the native annulus, so that the native leaflets are positioned between the anchors 80 and the body of the implant 70. When the implant 70 is in its final position, there may or may not be tension on the chordae, though the distal anchors 80 can be located between at least some of the chordae.

The proximal end 301 of the implant 70 will remain in the outer retention ring 42 after retraction of the capsule 106. The capsule 106 may surround the implant retention area and be retracted proximally to deploy the implant. As shown in FIG. 22B, once the distal end 303 of the implant 70 is fully expanded (or as fully expanded as possible at this point), the outer retention ring 42 can be individually withdrawn proximally with respect to the other assemblies, in particular relative to the inner assembly 18, to expose the inner retention member 40, thus beginning the expansion of the proximal end 301 of the implant 70. For example, in a tricuspid valve replacement procedure, after the distal or ventricular anchors 80 are positioned between at least some of the chordae tendineae and/or engage the native tricuspid valve annulus, the proximal end 301 of the implant 70 may be expanded within the right atrium.

The outer retention ring 42 can be moved proximally such that the proximal end 310 of the implant 70 can radially expand to its fully expanded configuration as shown in FIG. 22C. The implant 70 may be deployed to the valve. After expansion and release of the implant 70, the inner assembly 18, nose cone assembly 31, mid shaft assembly 21, and outer sheath assembly 22 can be simultaneously withdrawn proximally along or relative to the rail assembly 20 back to their original position. In some embodiments, they are not withdrawn relative to the rail assembly 20 and remain in the extended position. Further, the nose cone 28 can be withdrawn through the center of the expanded implant 70 and into the outer sheath assembly 22, such as by proximally translating the knob 216. The system 10 can then be removed from the patient.

In some embodiments, the implant 70 can be delivered under fluoroscopy so that a user can view certain reference points for proper positioning of the implant 70. Further, echocardiography can be used for proper positioning of the implant 70.

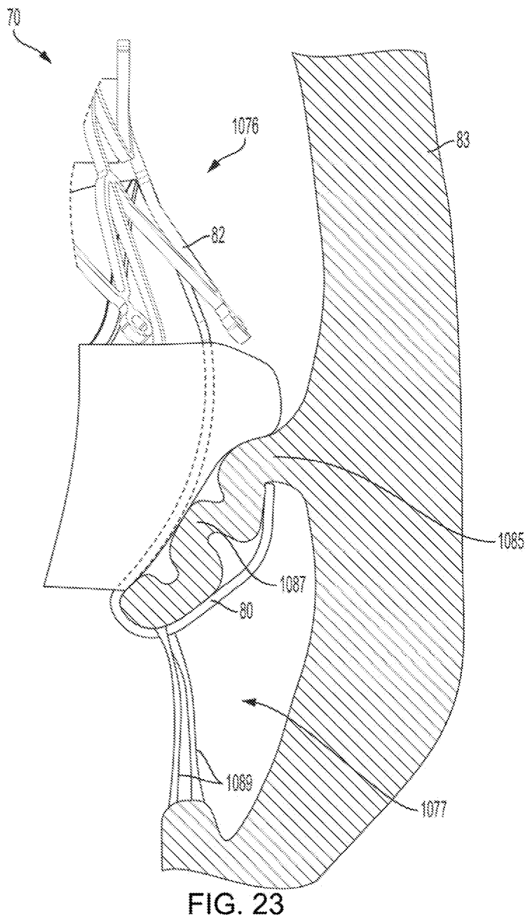

Reference is now made to FIG. 23 which illustrates a schematic representation of a portion of an embodiment of a replacement heart valve (implant 70) positioned within a native tricuspid valve of a heart 83. A portion of the native tricuspid valve is shown schematically and represents typical anatomy, including a right atrium 1076 positioned above an annulus 1085 and a right ventricle 1077 positioned below the annulus 1085. The right atrium 1076 and right ventricle 1077 communicate with one another through a tricuspid annulus 1085. Also shown schematically in FIG. 23 is a native tricuspid leaflet 1087 having chordae tendineae 1089 that connect a downstream end of the tricuspid leaflet 1087 to the papillary muscle of the right ventricle 1077. The portion of the implant 70 disposed upstream of the annulus 1085 (toward the right atrium 1076) can be referred to as being positioned supra-annularly. The portion generally within the annulus 1085 is referred to as positioned intra-annularly. The portion downstream of the annulus 1085 is referred to as being positioned sub-annularly (toward the right ventricle 1077).

As shown in FIG. 23, the replacement heart valve (e.g., implant 70) can be positioned so that the tricuspid annulus 1085 is located between the distal anchors 80 and the proximal anchors 82. In some situations, the implant 70 can be positioned such that ends or tips of the distal anchors 80 contact the annulus 1085 as shown, for example, in FIG. 23. In some situations, the implant 70 can be positioned such that ends or tips of the distal anchors 80 do not contact the annulus 1085. In some situations, the implant 70 can be positioned such that the distal anchors 80 do not extend around the leaflet 1087.

As illustrated in FIG. 23, the replacement heart valve or implant 70 can be positioned so that the ends or tips of the distal anchors 80 are on a ventricular side of the tricuspid annulus 1085 and the ends or tips of the proximal anchors 82 are on an atrial side of the tricuspid annulus 1085. The distal anchors 80 can be positioned such that the ends or tips of the distal anchors 80 are on a ventricular side of the native leaflets beyond a location where chordae tendineae 1089 connect to free ends of the native leaflets. The distal anchors 80 may extend between at least some of the chordae tendineae 1089 and, in some situations such as those shown in FIG. 23, can contact or engage a ventricular side of the annulus 1085. It is also contemplated that in some situations, the distal anchors 80 may not contact the annulus 1085, though the distal anchors 80 may still contact the native leaflet 1087. In some situations, the distal anchors 80 can contact tissue of the right ventricle 1077 beyond the annulus 1085 and/or a ventricular side of the leaflets.

Upon deployment of the implant 70 as desired, the deflection mechanisms disclosed in regard to FIGS. 13A-19B may be utilized to deflect the elongate shaft 12 to allow for removal of the elongate shaft 12 from the patient's heart.

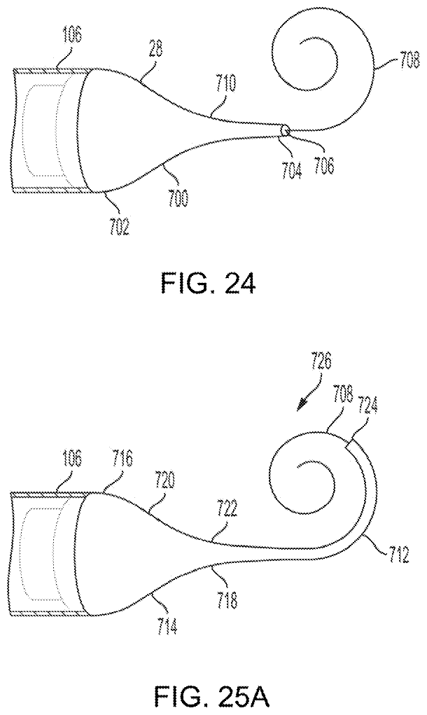

FIG. 24 illustrates a side perspective view of the nose cone 28 forming the tip of the elongate shaft 12. The nose cone 28 includes a tip body 700 that closes the end of the capsule 106 (shown in partial cross section) and is positioned distal of the capsule 106. The tip body 700 includes a proximal portion 702 and a distal portion 704 and tapers from the proximal portion 702 to the distal portion 704. An opening 706 is positioned at the distal portion 704 of the tip body 700 for the guide wire 708 to pass through. The distal portion 704 of the tip body 700 may include a stiff protruding section 710 that is tapered. The tapered profile of the stiff protruding section may allow for ease of entry into the patient's vasculature, and may assist to pass the tip of the elongate shaft 12 within the patient's vasculature.

Notably, however, the stiff protruding section 710 may interfere with or potentially damage a portion of the patient's body upon contact with the stiff protruding section 710. For example, if the nose cone 28 is passed into the right ventricle of the patient's heart, potentially the stiff protruding section 710 may impact and potentially puncture or otherwise damage the interior of the right ventricle. Notably, there is also a possibility of kinking with the guide wire 708 at the opening 706. The length of the stiff protruding section 710 may also inhibit maneuverability of the distal end of the elongate shaft 12.

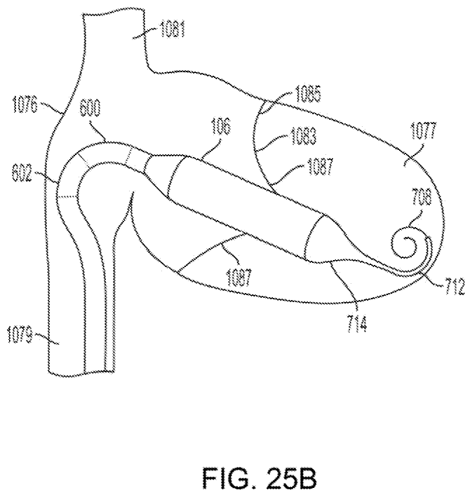

FIGS. 25A and 25B illustrate an embodiment of a distal tip of the elongate shaft 12 including a flexible sheath 712 that extends distally and is configured to bend about a portion of a guide wire 708. The distal tip may include a tip body 714 having a proximal portion 716 and a distal portion 718 and the distal tip may have an outer surface 720 that tapers in a direction from the proximal portion 716 to the distal portion 718. The tip body 714 may be positioned distal of the capsule 106 and may be positioned at and close the distal end of the capsule 106. The tip body 714 may be movable relative to the capsule 106 to allow an implant 70 enclosed by the capsule 106 to be deployed from the capsule 106.

The outer surface 720 may taper from a proximal portion 716 of the tip body 714 to a proximal portion 722 of the flexible sheath 712. The flexible sheath 712 may extend from the proximal portion 722 of the flexible sheath 712 to the distal end 724 of the flexible sheath 712. The flexible sheath 712 may have a cylindrical shape from the proximal portion 722 of the flexible sheath 712 to the distal end 724 of the flexible sheath 712.

The flexible sheath 712 may have a length that is configured to extend over a leading curve of the guide wire 708 for a guide wire 708 having a curved configuration 726 at the end of the guide wire 708. The flexible sheath 712 may thus cover the leading curve of the guide wire 708, to reduce the possibility of injury due to contact between the guide wire and a portion of the patient's body. FIG. 25B, for example, illustrates the distal tip within the patient's right ventricle 1077. The flexible sheath 712 is bent about the guide wire 708 when the guide wire 708 is positioned within the right ventricle. The flexible sheath 712 covers the portion of the guide wire 708 that may otherwise contact the interior wall of the patient's right ventricle 1077. Further, the flexible sheath 712 is flexible, to reduce the possibility of puncture or other interference with the interior wall of the patient's right ventricle 1077. The curvature of the flexible sheath 712 along the guide wire 708 additionally may reduce the possibility of kinking of the guide wire 708.

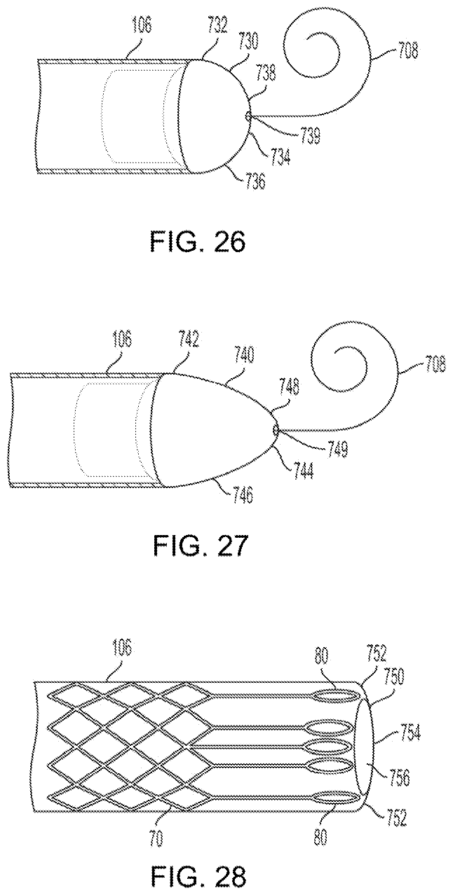

FIGS. 26-28 illustrate embodiments of distal tips of elongate shafts 12 that may reduce the distal profile of the elongate shafts 12. Such features may be utilized to allow the elongate shafts 12 to more easily navigate or be deflected in a variety of vascular geometries. For example, in the methods shown in FIGS. 20A-21, a reduced distal profile of the elongate shaft 12 may allow for greater maneuverability of the elongate shaft 12 within and towards the right atrium 1076.

FIG. 26 illustrates an embodiment of a distal tip of the elongate shaft 12 having a dome shape. The distal tip may include a tip body 730 having a proximal portion 732 and a distal portion 734 and may have an outer surface 736 that tapers in a direction from the proximal portion 732 to the distal portion 734. The tip body 730 may be positioned distal of the capsule 106 and may be positioned at and close the distal end of the capsule 106. The tip body 730 may be movable relative to the capsule 106 to allow an implant 70 enclosed by the capsule 106 to be deployed from the capsule 106. The dome shaped tip body may form a convex profile of the distal end 738 of the distal tip. The outer surface 736 may be convex from the proximal portion 732 of the tip body 730 to the distal end 738 of the tip body 730. The tip body 730 may include an opening 739 at its distal end 738 for a guide wire 708 to pass through.

FIG. 27 illustrates an embodiment of a distal tip of the elongate shaft 12 having a parabolic shape. The distal tip may include a tip body 740 having a proximal portion 742 and a distal portion 744 and may have an outer surface 746 that tapers in a direction from the proximal portion 742 to the distal portion 744. The tip body 740 may be positioned distal of the capsule 106 and may be positioned at and close the distal end of the capsule 106. The tip body 740 may be movable relative to the capsule 106 to allow an implant 70 enclosed by the capsule 106 to be deployed from the capsule 106. The parabolic shaped tip body may form a convex profile of the distal end 748 of the distal tip. The outer surface 746 may be convex from the proximal portion 742 of the tip body 740 to the distal end 748 of the tip body 740. The tip body 740 may include an opening 749 at its distal end 748 for a guide wire 708 to pass through.

FIG. 28 illustrates an embodiment in which the distal end 750 of the capsule 106 forms the distal tip of the elongate shaft 12. The distal end 750 of the capsule may include a rounded portion 752 that extends over the distal ends (or anchors 80) of the implant and may provide a smooth profile for the distal tip of the elongate shaft 12. The distal tip accordingly may comprise an atraumatic rounded tip. The capsule may include a portion 754 having a planar profile at the leading edge of the capsule 106. The portion 754 may include an opening or port 756 for the implant to be deployed from.

The capsule 106 may be configured to have a distal end 705 that is elastic, and may conform to the shape of the implant 70 positioned within the capsule 106. A tie-layer or the like may be added to the capsule 106 to provide elasticity of the capsule 106 against the implant 70. The capsule 106 may include an ePTFE tip with a low durometer elastic tie layer for example. Upon deployment of the implant 70, the implant may be advanced distally from the capsule 106 through the port 756, with the rounded portion 752 of the distal end 750 expanding to accommodate the distal movement of the implant 70. The port 756 or an opening in the distal end 705 of the capsule 106 may be configured to allow a guide wire 708 to pass through.

In the embodiment shown in FIG. 28, a separate tip body may not be present at the distal tip of the elongate shaft 12, thus reducing the distal profile of the elongate shaft 12.

One or more features of the embodiments of distal tips of FIGS. 25A-28 may be utilized solely or with any other embodiment of delivery system or other system, or other methods, disclosed herein.

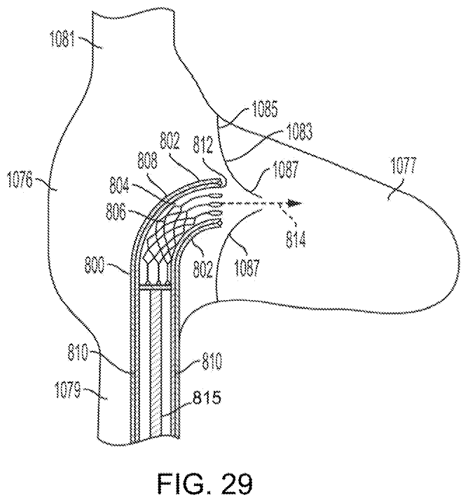

FIG. 29 illustrates an embodiment of an elongate shaft 800 that is configured with a wall 802 surrounding a channel 804 for an implant 806 to be passed through for deployment of the implant 806. The wall 802 may be configured to have a bend 808 that defines a bend in the channel 804 during the deployment of the implant 806.

The wall 802 may be configured to be steerable, and a control mechanism may be utilized to steer the wall 802. For example, pull tethers 810 or other forms of control mechanisms may be utilized to steer the wall 802, to control the direction of bend of the wall 802, and particularly to direct an opening or port 812 for the implant 806 to be passed through to a desired orientation.

In one embodiment, the wall 802 may not be steerable, but the wall may have a bend preformed by the wall 802 in a desired orientation.

The channel 804 may be a deployment channel for the implant 806 to be deployed from. The channel 804 may be configured to retain the implant 806 and may comprise an implant retention area. The channel 804 may be configured to retain the implant 806 upon approach and entry of the right atrium 1706 or other portion of the patient's heart or vasculature.

The implant 806 may be configured to be a flexible implant, configured to bend in a direction transverse to an axial dimension 814 of the implant 806. As such, the implant 806 may be configured to bend within the channel 804 in the direction transverse to the axial dimension 814 of the implant 806 for deployment of the implant 806. A deployment device, such as a push shaft 815 may be utilized to push the implant 806 from the port 812 for deployment. Other forms of deployment devices, such as expandable balloons may be utilized as desired.

The implant 806 may be an expandable implant, and may be self-expanding, for deployment to the desired portion of the patient's body. The implant 806 may be configured similarly as the implant 70, yet may be configured to bend in a direction transverse to an axial dimension 814 of the implant 806 when passing through the bent deployment channel. Such a configuration may be provided by the frame of the implant 70 being made thinner to allow for greater flexibility in a transverse direction.

Components of the elongate shaft 12 may be utilized with the elongate shaft 800, including use of an outer sheath assembly, a mid shaft assembly, a rail assembly, an inner shaft assembly, and a nose cone assembly. Any or all of the assemblies may be utilized to perform or assist with deployment of the implant 806. The deflection mechanisms disclosed herein may also be utilized. One or more features of the elongate shaft 800 may be utilized solely or with any other embodiment of delivery system or other system, or other methods, disclosed herein.

The use of the wall 802 having a bend 808 that defines a bend in the channel 804 during the deployment of the implant 806, may provide benefits including a reduced transverse profile of the elongate shaft 800. For example, as shown in FIGS. 20A-21, the capsule 106 of the elongate shaft 12 may form a relatively large turning radius for the elongate shaft 12 about the bend portion 600. The use of a bend in the channel 804 may allow for a reduced transverse profile of the elongate shaft 800, with a relatively smaller turning radius. The port 812 accordingly may be moved proximate the tricuspid valve 1083 for deployment of the flexible implant 806, with the elongate shaft 800 having a reduced transverse profile. The implant may be passed through a bent deployment channel to deploy the implant (which may be a prosthetic tricuspid valve).

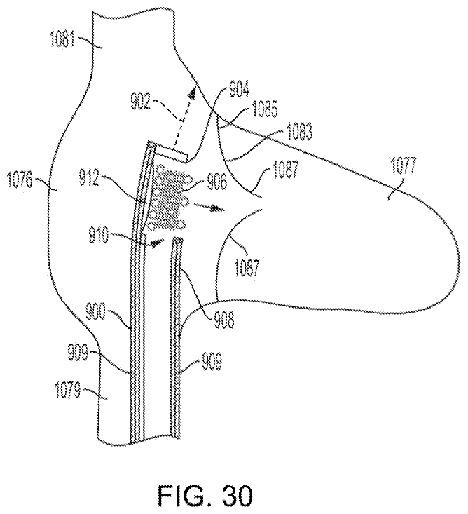

FIG. 30 illustrates an embodiment of an elongate shaft 900 having an axial dimension 902 and having a port 904 for an implant 906 to be deployed from in a direction transverse to the axial dimension 902. The elongate shaft 900 may include a side wall 908 and the port 904 may be positioned on the side wall 908.

The side wall 908 may be configured to be steerable, and a control mechanism may be utilized to steer the side wall 908. For example, pull tethers 909 or other forms of control mechanisms may be utilized to steer the side wall 908, to direct the port 904 to a desired orientation.

The elongate shaft 900 may include an implant retention area 910 for retaining the implant 906. The implant 906 may be configured to be deployed in the axial dimension of the implant 906, exiting through the port 904 in the axial dimension of the implant 906. The implant 906 may be configured to be compressed in the axial dimension of the implant 906 prior to deployment.

A deployment mechanism may be utilized to deploy the implant 906 from the port 904. The deployment mechanism may include an inflatable body 912 configured to push the implant 906 out of the port 904 as shown in FIG. 30, or in other embodiments, other forms of deployment mechanisms may be utilized. The implant may be deployed through the port 904 in a direction transverse to the axial dimension of the elongate shaft.

The implant 906 may be an expandable implant, and may be self-expanding, for deployment to the desired portion of the patient's body. The implant 906 may be configured similarly as the implant 70, yet may be configured to be compressed in the axial dimension of the implant 906.

Components of the elongate shaft 12 may be utilized with the elongate shaft 900, including use of an outer sheath assembly, a mid shaft assembly, a rail assembly, an inner shaft assembly, and a nose cone assembly. Any or all of the assemblies may be utilized to perform or assist with deployment of the implant 906. The deflection mechanisms disclosed herein may also be utilized. One or more features of the elongate shaft 900 may be utilized solely or with any other embodiment of delivery system or other system, or other methods, disclosed herein.

The use of the elongate shaft 900 having an axial dimension 902 and having a port 904 for an implant 906 to be deployed from in a direction transverse to the axial dimension 902, may provide benefits including a reduced transverse profile of the elongate shaft 900. For example, as shown in FIGS. 20A-21, the capsule 106 of the elongate shaft 12 may form a relatively large turning radius for the elongate shaft 12 about the bend portion 600. The use of a port 904 for an implant 906 to be deployed from in a direction transverse to the axial dimension 902 may allow for a reduced transverse profile of the elongate shaft 900. The port 904 accordingly may be moved proximate the tricuspid valve 1083 for deployment of the implant 906, with the elongate shaft 900 having a reduced transverse profile.

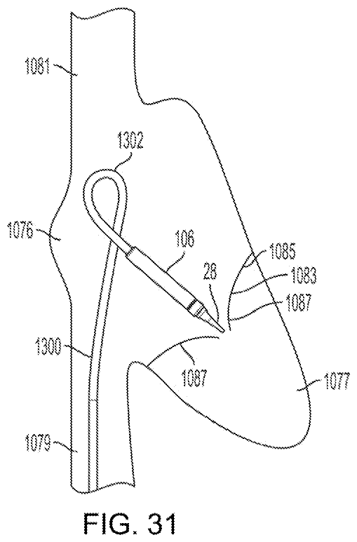

FIG. 31 illustrates an embodiment of an elongate shaft 1300 that is configured to bend more than 180 degrees to form a loop 1302. The shaft 1300 may be configured similarly as the elongate shaft 12, yet may be configured to bend more than 180 degrees to form the loop 1302. Such a feature may be provided by a control mechanism that is configured to cause the bend of greater than 180 degrees, such as a push shaft that extends along the outer diameter of the shaft 1300 and applies a distal force to cause the shaft 1300 to bend more than 180 degrees. Other mechanisms may be utilized as well. The elongate shaft 1300 may thus form a loop 1302, which may be positioned in a desired location within the patient's body.

For example, as shown in FIG. 31, the loop 1302 may be positioned within the right atrium 1076, in an embodiment in which the implant 70 is to be deployed to the tricuspid valve 1083. The loop 1302 may be positioned within the right atrium 1076 to allow for greater clearance of the distal end of the capsule 106 from the wall of the right atrium 1076. The loop 1302 may be directed in the atrial direction, away from the tricuspid valve 1083. The elongate shaft is configured to bend at a bend portion of the elongate shaft, with the implant retention area being positioned distal of the bend portion.

The elongate shaft is bent more than 180 degrees to form a loop at least partially within the patient's right atrium. The degree of bend of the elongate shaft 1300 may vary as desired, for example the degree of bend may be more than 200 degrees in one embodiment, may be more than 230 degrees in one embodiment, may be more than 250 degrees in one embodiment, and may be more than 270 degrees in one embodiment. Other degrees of bend may be utilized as desired. One or more features of the elongate shaft 1300 may be utilized solely or with any other embodiment of delivery system or other system, or other methods, disclosed herein.

Components of the elongate shaft 12 may be utilized with the elongate shaft 1300, including use of an outer sheath assembly, a mid shaft assembly, a rail assembly, an inner shaft assembly, and a nose cone assembly. Any or all of the assemblies may be utilized to perform or assist with deployment of the implant 70 retained by the implant retention area. The deflection mechanisms disclosed herein may also be utilized.

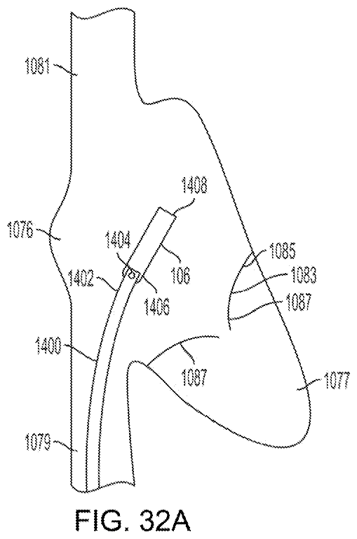

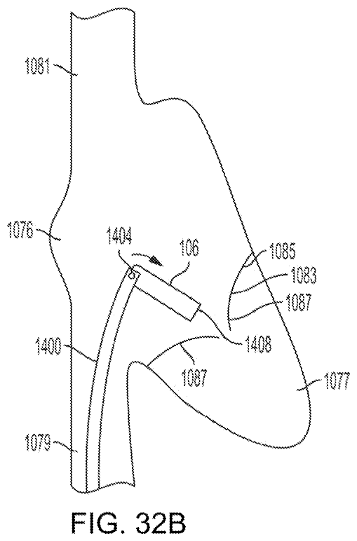

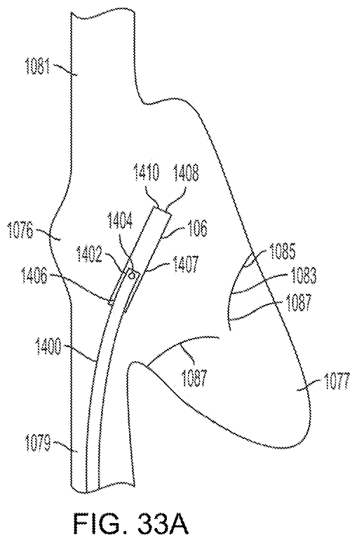

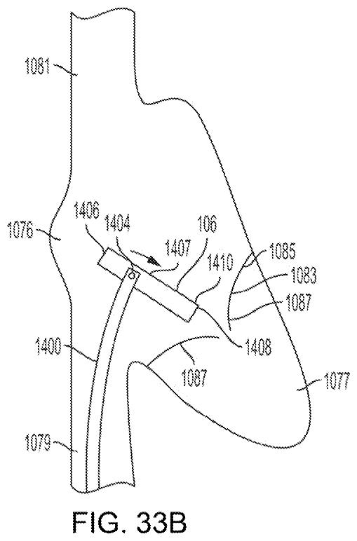

FIGS. 32A-33B illustrate embodiments of elongate shafts including a hinge that couples the capsule to a portion of the elongate shaft. FIG. 32A, for example, illustrates an elongate shaft 1400 having a distal portion 1402 with a hinge 1404. The hinge 1404 couples to a proximal portion 1406 of a capsule 106. The capsule 106 is configured to rotate about the hinge 1404 to place the port 1408 of the capsule 106 in the desired orientation relative to the tricuspid valve 1083. FIG. 32B, for example, illustrates the capsule 106 rotated about the hinge 1404 with the port 1408 oriented towards the tricuspid valve 1083.

FIG. 33A illustrates an embodiment in which the hinge 1404 at the distal portion 1402 of the elongate shaft 1400 couples to the capsule 106 at a central portion 1407 of the capsule that is positioned between the proximal portion 1406 of the capsule 106 and the distal portion 1410 of the capsule 106. The capsule accordingly may be pivoted about the hinge 1404 to place the port 1408 of the capsule 106 in the desired orientation relative to the tricuspid valve 1083. FIG. 33B for example, illustrates the capsule 106 rotated about the hinge 1404 with the port 1408 oriented towards the tricuspid valve 1083.

The capsules 106 shown in FIGS. 32A-33B may be rotated about the hinge 1404 through use of a control mechanism, which may comprise push or pull shafts or other devices configured to control rotation of the capsule 106. The implant may be configured to be deployed from the capsule 106 by a deployment mechanism, which may comprise an inflatable body or the like for deploying the implant from the capsule 106.

The capsules 106 may be configured to rotate about the hinge 1404 to a variety of angles, including between zero degrees and 360 degrees, or more, as desired. The capsules 106 may be rotated to provide the desired orientation of the port 1408 of the capsule 106, for example in a desired orientation relative to a tricuspid valve 1083 or other delivery location.

The hinge 1404 may comprise a pin extending through an aperture, or may comprise other forms of hinges as desired.

Components of the elongate shaft 12 may be utilized with the elongate shaft 1400, including use of an outer sheath assembly, a mid shaft assembly, a rail assembly, an inner shaft assembly, and a nose cone assembly. Any or all of the assemblies may be utilized to perform or assist with deployment of the implant 70 retained by the implant retention area. The deflection mechanisms disclosed herein may also be utilized. One or more features of the elongate shaft 1400 may be utilized solely or with any other embodiment of delivery system or other system, or other methods, disclosed herein.

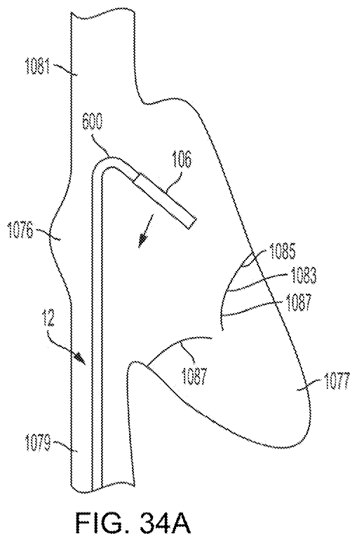

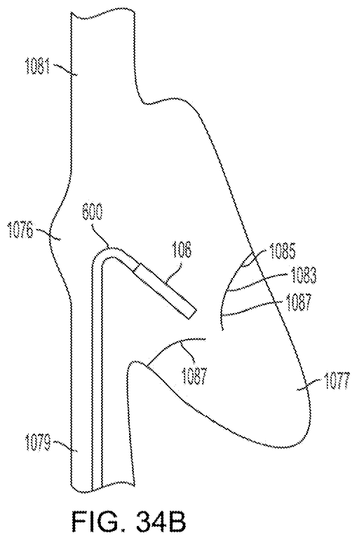

FIGS. 34A—B illustrate a method that may be utilized for deployment of an implant from the elongate shaft 12. Referring to FIG. 34A, the method may include positioning the capsule 106 of the elongate shaft 12 within the right atrium 1076 of the patient's heart. The bend portion 600 may then be utilized to deflect the capsule 106 to a direction. The elongate shaft 12 may then be translated proximally, for example by retracting the elongate shaft 12 from the patient's heart, to position the capsule 106 in the desired orientation relative to the tricuspid valve 1083.

FIG. 34B for example, illustrates the elongate shaft 12 having been retracted in a proximal direction to place the capsule 106 in a desired position relative to the tricuspid valve 1083. The implant may then be deployed from the capsule 106 for implantation to the tricuspid valve 1083 using methods disclosed herein. The method of FIGS. 34A-B may be utilized solely or with any other embodiment of delivery systems, other systems, or methods disclosed herein.

Various other methods of deploying implants or utilizing the systems and apparatuses disclosed herein may be utilized.

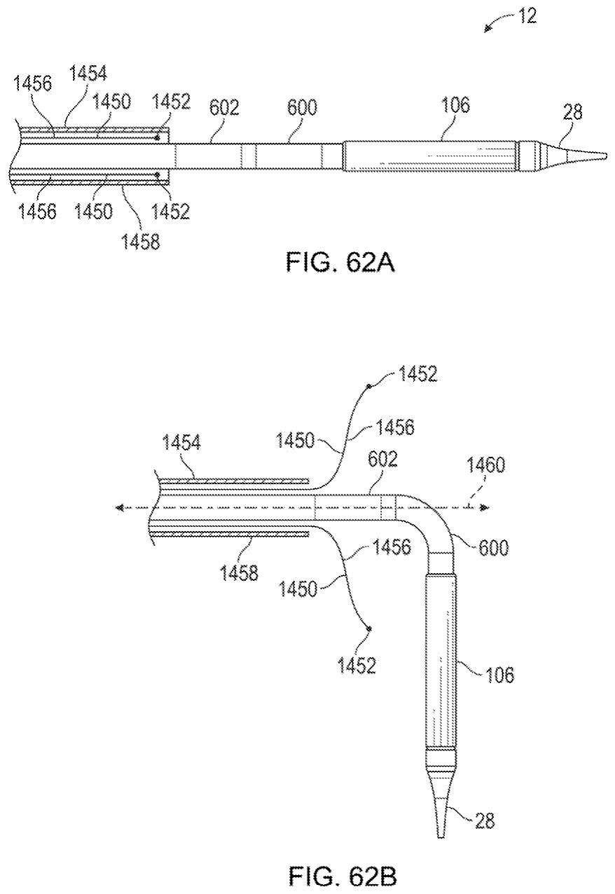

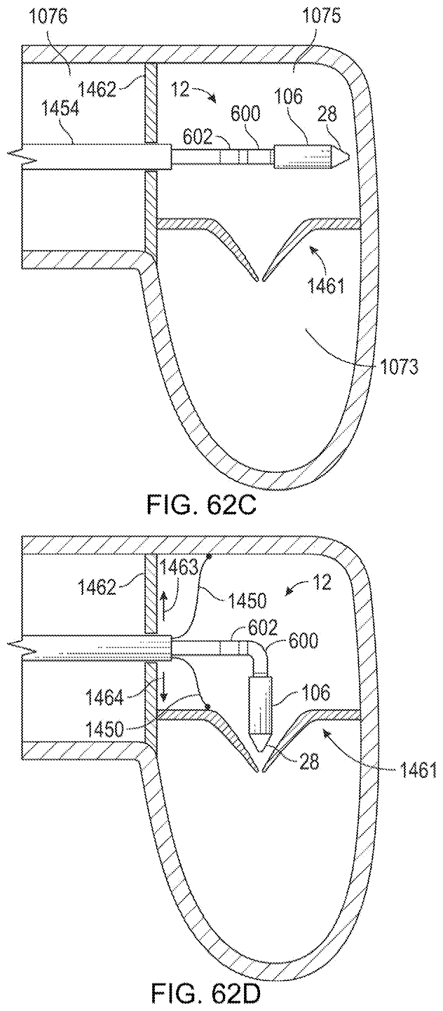

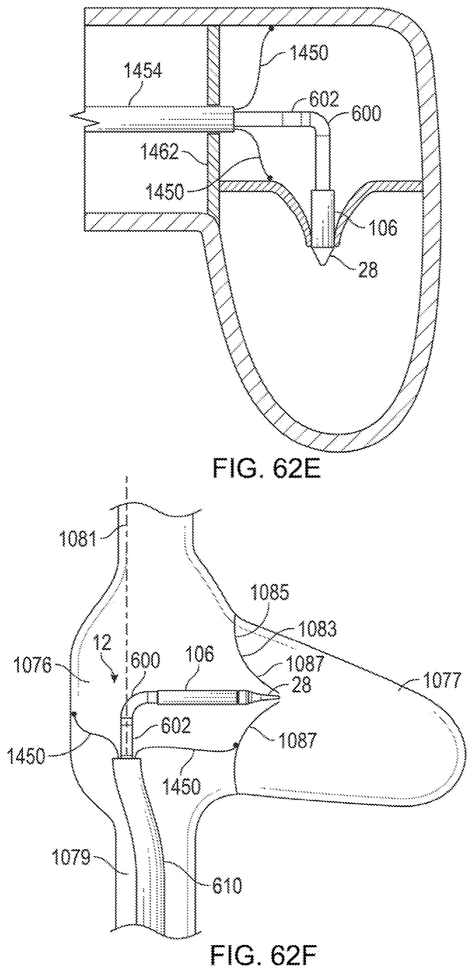

FIGS. 62A-64C illustrate embodiments utilizing one or more support bodies configured to extend radially outward from an outer surface of the elongate shaft 12 and contact an external surface to resist deflection of the elongate shaft transverse to an axis that the elongate shaft 12 extends along. The embodiments may be utilized with any other embodiment disclosed herein.

FIG. 62A, for example, illustrates an embodiment in which one or more support bodies 1450 in the form of arms may be utilized. The support bodies 1450 are shown in an undeployed, unexpanded, or linearized configuration in FIG. 62A in which the support bodies 1450 are collapsed against the outer surface of the elongate shaft 12. The support bodies 1450 may extend from distal tips 1452 of the support bodies 1450 proximally to the position of the handle 14 or to another position for access exterior of the patient's body.

The support bodies 1450 may be held in the undeployed, unexpanded, or linearized configuration shown in FIG. 62A by a sheath 1454 that extends along the outer surface of the elongate shaft 12 and over the elongate shaft 12 and support bodies 1450. The sheath 1454, for example, may be configured similarly as the sheath 51 shown in FIG. 1 or shown as sheath 610 in FIG. 13A as examples. In embodiments, the sheath 1454 may be configured to slide proximally and/or distally along the outer surface of the elongate shaft 12 to uncover or cover the support bodies 1450 as desired. A proximal portion of the sheath 1454 for example may be controlled to move the sheath 1454 proximally or distally.

The support bodies 1450 may extend to the distal tips 1452 of the support bodies 1450. Each support body 1450 may have an intermediate portion 1456 between the distal tip 1452 and a proximal portion of the support body 1450 that may be configured to extend radially outward from the sheath 1454 and the outer surface of the elongate shaft 12. Each support body 1450 may be shaped to extend radially outward from the outer surface of the elongate shaft 12 and in embodiments may be biased to extend radially outward from the outer surface of the elongate shaft 12. For example, the support bodies 1450 may comprise a shape memory material that may be pre-shaped to extend radially outward. The shape memory material may comprise nitinol or another form of shape memory material. In embodiments, the support bodies 1450 may be made from other materials such as stainless steel or another material.

The support bodies 1450 may each be configured to contact an external surface. The external surface may comprise a portion of a patient's vasculature, including a portion of a patient's heart. For example, in embodiments, the support bodies 1450 may be configured to contact atrial walls (which may include an interatrial septum) or other portions of the patient's heart. The support bodies 1450 may be configured to be atraumatic. The intermediate portions 1456 and distal tips 1452, for example, may each be rounded or smooth to reduce the possibility of damage to a heart wall.

The support bodies 1450 may each be sufficiently stiff to reduce deflection of the elongate shaft 12 in a direction transverse to an axis that the elongate shaft 12 extends along. The support bodies 1450, however, may be flexible to extend radially outward from an unexpanded configuration (as shown in FIG. 62A) to an expanded configuration (as shown in FIG. 62B). The support bodies 1450 may deflect outward by the sheath 1454 being retracted proximally or the support bodies 1450 being advanced distally relative to the distal end 1458 of the sheath 1454.

FIG. 62B illustrates the support bodies 1450, for example, having been advanced distally relative to the sheath 1454. The support bodies 1450 extend outward radially in the expanded configuration shown in FIG. 62B. The intermediate portions 1456 of the support bodies 1450 protrude from the distal end 1458 of the sheath 1454 outward to the distal tips 1452 of the support bodies 1450. The support bodies 1450 are positioned to resist deflection of the elongate shaft 12 transverse to an axis 1460 that the elongate shaft 12 extends along.

In embodiments, the support bodies 1450 may be advanced distally and/or the sheath 1454 may be retracted proximally to expand the support bodies 1450.

FIGS. 62C—E illustrate an exemplary method of utilizing a delivery apparatus utilizing the support bodies 1450. FIG. 62C, for example, illustrates the delivery apparatus may be utilized to deliver an implant to a mitral valve 1461. The elongate shaft 12 of the delivery apparatus for example, may be passed transseptally, through the interatrial septum 1462 between the right atrium 1076 and the left atrium 1075. A puncture, for example, may be made at the interatrial septum 1462 that allows the elongate shaft 12 to pass through the interatrial septum 1462. The support bodies 1450 may remain in the unexpanded configuration, covered by the sheath 1454 at this point. The delivery apparatus is positioned within the left atrium 1075.

FIG. 62D illustrates that the capsule 106 surrounding the implant retention area may be deflected in the ventricular direction, towards the left ventricle 1073 via the bend portion 600 for example. One or more other bend portions, including bend portion 602 may be utilized to align the capsule 106 as desired with respect to the mitral valve 1461. For example, the bend portion 602 may deflect the capsule in a plane extending transverse to the plane of bend of the bend portion 600 according to methods disclosed herein. Various directions of deflection may be utilized.

With the capsule 106 aligned in position, a depth of the capsule 106 may be increased in the ventricular direction utilizing methods disclosed herein. An increase in depth in the ventricular direction, however, may result in a force applied in an atrial direction 1463 (marked in FIG. 62D) upon the elongate shaft 12. Further, a reduction of depth towards the atrial direction may result in a force applied in a ventricular direction 1464 (marked in FIG. 62D) upon the elongate shaft 12. Such forces may result from the movement of the capsule 106 or may result from contact of the capsule 106 with a structure, such as chordae or the valve leaflets of the mitral valve 1461. The force upon the elongate shaft 12 may provide stress upon the puncture of the interatrial septum 1462. In embodiments, the stress may increase the size of the puncture of the interatrial septum 1462, which may be undesirable, and may increase the time for the puncture to seal or may reduce the likelihood of the puncture sealing.

To reduce the deflection of the elongate shaft 12, and the possible stress to the interatrial septum 1462, the support bodies 1450 may be expanded radially outward to contact the walls of the patient's heart. FIG. 62D, for example, illustrates the support bodies 1450 expanded, in a configuration as shown in FIG. 62B. The support bodies 1450 move to the expanded state from the unexpanded state. The distal tips 1452 and/or intermediate portions 1456 of the support bodies 1450 may contact the atrial wall to support the elongate shaft 12. The support bodies 1450 may be positioned to resist a force in the atrial direction 1463 and/or the ventricular direction 1464 as desired. In embodiments, other directions (e.g., transverse to the atrial direction 1463 and/or ventricular direction 1464) may be utilized as desired. The atrial wall may comprise opposing portions of the atrial wall as shown in FIG. 62D, or may comprise a wall of the interatrial septum in embodiments.

The support bodies 1450 may be positioned proximal of the bend portions 600, 602 or may be in another location as desired. The support bodies 1450 may remain in position during an increase in depth of the capsule 106 or another deployment procedure performed by the elongate shaft 12. FIG. 62E, for example, illustrates the support bodies 1450 in position as the depth of the capsule 106 is increased in the ventricular direction.

Upon deployment of the implant, the support bodies 1450 may be retracted to the undeployed, unexpanded, or linearized configuration as shown in FIG. 62A and then withdrawn from the patient's body. The support bodies 1450 may be withdrawn along with the elongate shaft 12.

In embodiments, the sheath 1454 may not be utilized and the support bodies 1450 may be coupled directly to the elongate shaft 12 and may extend radially outward from the elongate shaft 12. A separate control mechanism may be utilized to control deployment of the support bodies 1450 in such an embodiment.

The support bodies 1450 may beneficially reduce deflection of the elongate shaft 12 and may reduce stress upon the interatrial septum 1462. As such, the precision of the deflection and depth control of the capsule 106 may be improved due to the reduced possibility of undesired deflection of the elongate shaft 12. Further, the reduced stress to the interatrial septum 1462 may reduce the possibility of an undesired increase in size of the puncture of the interatrial septum 1462. Such a feature may reduce the time for the puncture to seal or enhance the likelihood of the puncture sealing. A smaller puncture of the interatrial septum may reduce the likelihood of requiring an occluder to be utilized to seal the puncture following the deployment of the heart valve implant. As such, reduced steps for an implant deployment procedure may result.

The support bodies 1450 may further be utilized in deployment to other locations within the patient's body. FIG. 62F, for example, illustrates an embodiment in which the support bodies 1450 are utilized for deployment to the tricuspid valve. The delivery apparatus is positioned within the right atrium 1076. The support bodies 1450 may extend radially outward from the elongate shaft 12 and contact the right atrial wall. Other positions of contact of the patient's vasculature may be utilized, such as the inferior vena cava 1079 or the superior vena cava 1081 as desired.

The form of the support bodies may be varied in embodiments.

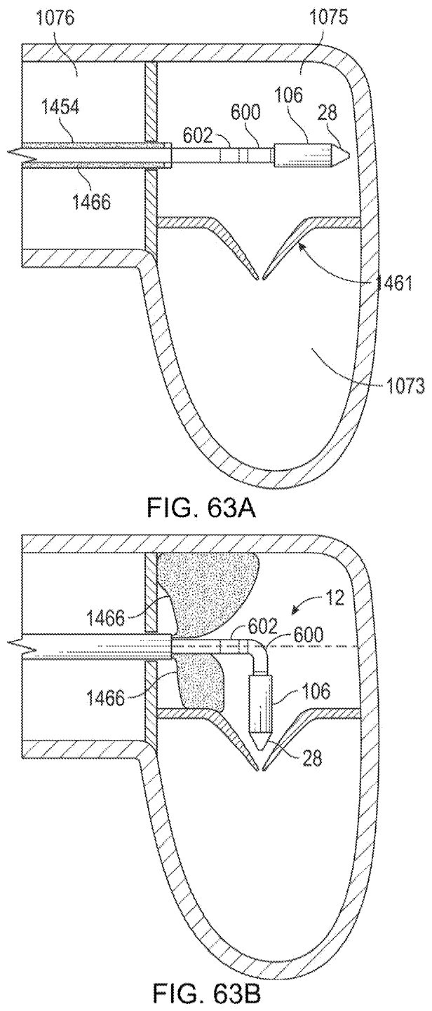

FIGS. 63A-B, for example, illustrate an embodiment in which the support body 1466 comprises an inflatable body. The support body 1466 may be configured to be inflated with fluid or the like to move from an unexpanded, undeployed, or linearized configuration as shown in FIG. 63A to an expanded or deployed configuration as shown in FIG. 63B. FIG. 63A illustrates the support body in an unexpanded or deflated configuration with the sheath 1454 extending over the support body 1466. The support body 1466 may then be inflated via a fill lumen or the like to the expanded or inflated configuration as shown in FIG. 63B.

Referring to FIG. 63B, the support body 1466 may contact the atrial wall to resist deflection of the elongate shaft 12 in a similar manner as discussed regarding the support body 1450 shown in FIG. 62E or FIG. 62F. In embodiments, one or more inflatable bodies may be utilized. The inflatable bodies may have a rectangular shape, or other shape (e.g., spheroid) or may have another shape such as a disk in embodiments. Further, the configuration of the inflatable bodies may comprise membranes, or may comprise a mesh body, or may have another form in embodiments.

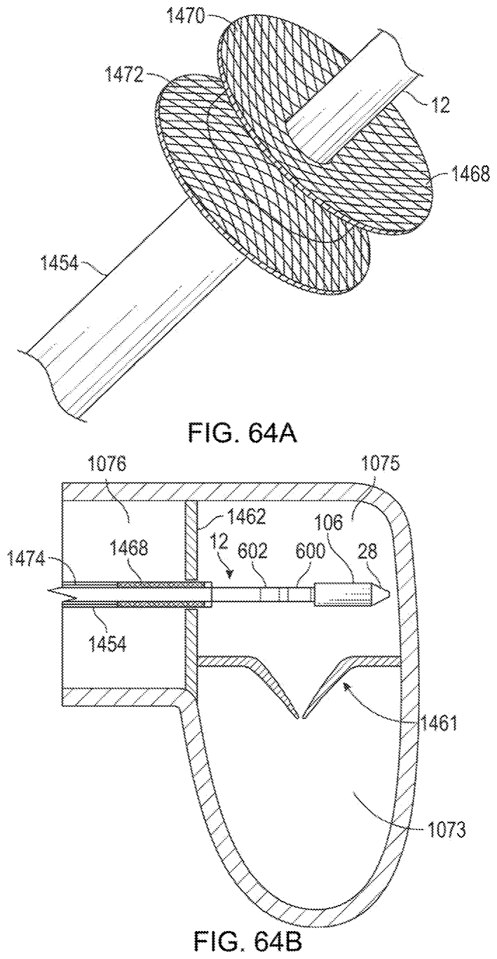

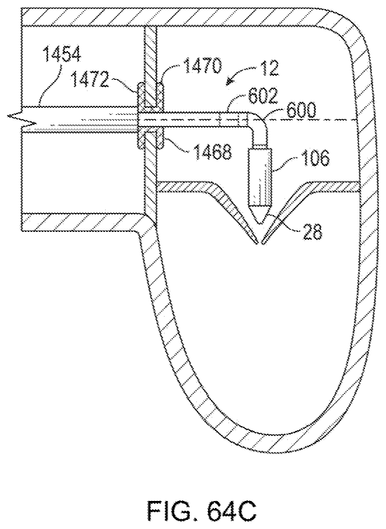

FIGS. 64A-B illustrate an embodiment in which the support body 1468 comprises a mesh configured to extend radially outward from the elongate shaft 12. The mesh may be configured as a plurality of disks 1470, 1472 that each extend radially outward from the elongate shaft 12. The disks 1470, 1472 may be configured to be positioned on opposite sides of a puncture of an interatrial septum, although other locations may be utilized in embodiments. In embodiments, a single disk may be utilized or a greater number of disks (e.g., three disks, or four disks) may be utilized as desired.

The support body 1468 may be configured as one or more occluders that are configured to seal a puncture of the interatrial septum. As such, the support body may reduce fluid flow between the atria and support the elongate shaft 12 from deflection in embodiments.

FIG. 64B, for example, illustrates the support body 1468 in an unexpanded, undeployed, or linearized configuration. A sheath 1454 extends over the support body 1468. A tether 1474 may couple the support body 1468 to the sheath 1454 or the elongate shaft 12. The support body 1468 may be placed in the desired position relative to the puncture in the interatrial septum and the deployment site.

FIG. 64C illustrates the support body 1468 deployed, and extending radially outward from the elongate shaft 12. The surface area of the disks 1470, 1472 against the interatrial septum may reduce the possibility of deflection of the elongate shaft 12. A disk 1470 may be positioned in the left atrium and another disk 1472 may be positioned in the right atrium. The position and configuration of the disks may be varied in embodiments. The disks 1470, 1472 for example, may be made of a mesh material configured to expand upon deployment. The material may comprise a shape memory material such as nitinol or another form of shape memory material. In embodiments, the disks may comprise inflatable bodies or may comprise other forms of occluders.

The support body 1468 may either be retracted and withdrawn upon deployment of the heart valve implant, or may remain in place within the puncture of the interatrial septum. The support body 1468 may remain in place as an occluder following deployment.

In embodiments, various other forms of mesh bodies and disks may be utilized as support bodies herein. The support bodies may be used for deployment to a mitral valve or a tricuspid valve, or another valve as desired. One or more features of the embodiments of support bodies may be utilized with any other embodiment of delivery system, or other system, or methods, disclosed herein.

The implants disclosed herein may be utilized with anchors that are configured to be secured within a portion of a patient's body. The anchors may serve to further secure the implants to the desired implantation location within the patient's heart. The implants may comprise prosthetic heart valves, and particularly may comprise prosthetic heart valves configured for implantation within a patient's tricuspid valve annulus 1085. The implants may comprise prosthetic tricuspid heart valves and may comprise implants that are disclosed herein.

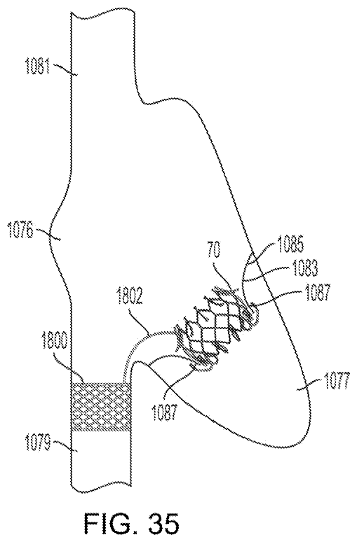

FIGS. 35-38B illustrate embodiments of anchors that may be utilized with implants disclosed herein. FIG. 35, for example, illustrates the implant 70 in position within the tricuspid valve annulus 1085 of the patient's heart. The implant 70 includes prosthetic valve flaps to replace the native valve flaps. An anchor 1800 may be utilized that is configured to be secured within the inferior vena cava 1079 of the patient's heart. The anchor 1800 may comprise a stent that is secured within the inferior vena cava 1079. A tether 1802 may couple from the anchor 1800 to the implant 70 to secure the implant in position within the tricuspid valve annulus 1085. The tether 1802 may be rigid, to resist a force in the atrial direction applied to the implant 70. In one embodiment, the anchor 1800 may be positioned within the superior vena cava 1081 of the patient's heart, alternatively or in combination with an anchor in the inferior vena cava 1079. An atrial ball anchor may be utilized in certain embodiments.

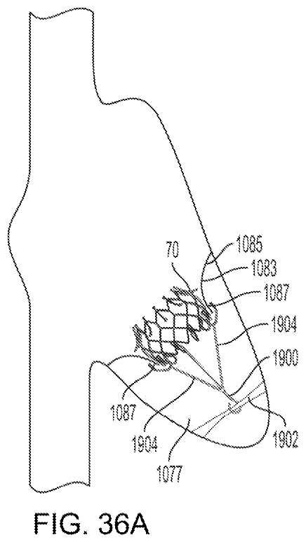

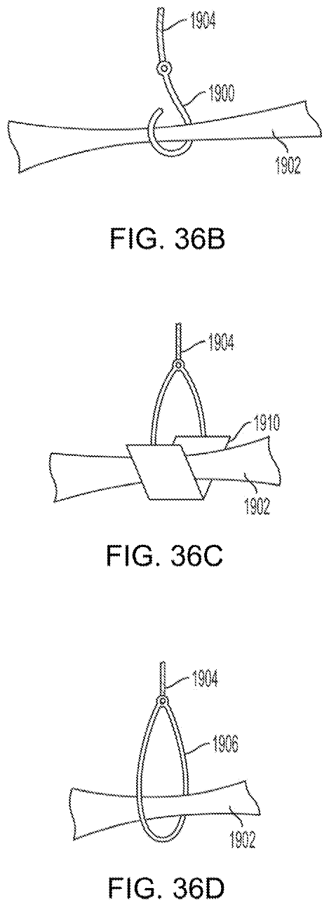

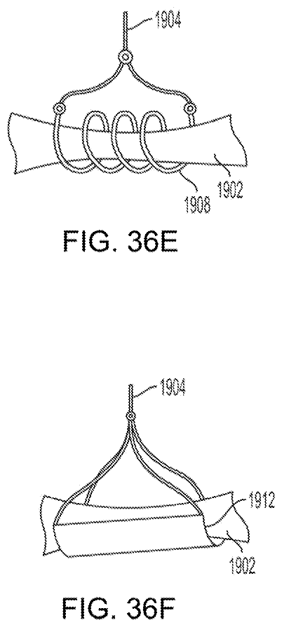

FIG. 36A illustrates an embodiment in which an anchor 1900 is configured to be secured to a moderator band 1902 of the patient's right ventricle 1077. The anchor 1900 may couple to one or more tethers 1904 that couple to the implant 70. The tethers 1904 may be configured to resist a force applied to the implant 70 in the atrial direction, to secure the implant 70 within the tricuspid valve annulus 1085.

The anchor 1900 may have a variety of forms. The anchor 1900 may comprise a hook as shown in FIGS. 36A and 36B. In one embodiment, the anchor 1900 may have the form of a loop 1906 as shown in FIG. 36D, or multiple loops 1908 (one or more loops) as shown in FIG. 36E. In one embodiment, the anchor 1900 may have the form of a cover 1910 for covering a portion of the moderator band 1902 as shown in FIG. 36C. The cover 1910 may have a V-shaped configuration as shown in FIG. 36C, or may have a U-shaped configuration as shown with the cover 1912 in FIG. 36F.

The anchors may be deployed to the moderator band 1902 during the process of implantation of the implant 70, or in a separate process in which the tether 1904 is coupled between the anchor and the implant 70. Additional forms of anchors may include barbs or expandable bodies that are expanded over a portion of the moderator band 1902 to secure the anchor to the moderator band 1902.

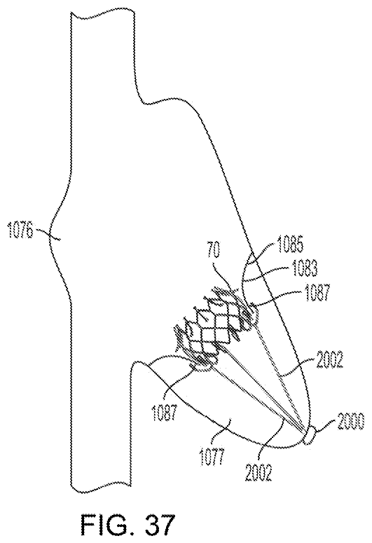

FIG. 37 illustrates an embodiment in which an anchor 2000 may be secured to a wall of the patient's right ventricle 1077. For example, the anchor 2000 may comprise an expandable body that is passed through a puncture in the wall in a compressed or undeployed state and placed on an exterior surface of the wall of the right ventricle 1077. The expandable body may be expanded to have a size larger than the size of the puncture to prevent the anchor 2000 from passing back through the puncture. One or more tethers 2002 may couple the anchor 2000 to the implant 70 to secure the implant in position within the tricuspid valve annulus 1085. The anchor 2000 in other embodiments may have other forms, including barbs or hooks for coupling to the wall of the right ventricle 1077. The anchor 2000 may be deployed and secured to the wall of the right ventricle 1077 during the process of implantation of the implant 70, or in a separate process in which the tether 2002 is coupled between the anchor 2000 and the implant 70.

The embodiments disclosed herein may be deployed within a patient's tricuspid valve annulus, and an anchor may be deployed to a portion within a patient's body. A tether may be provided coupling the prosthetic heart valve to the anchor. The anchor may be coupled to the prosthetic heart valve with the tether.

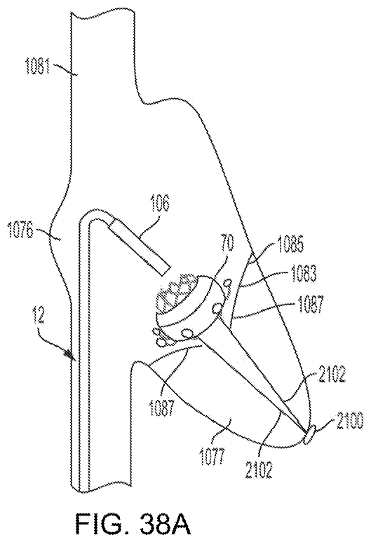

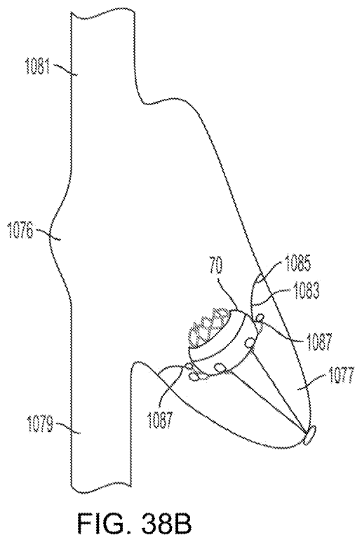

FIG. 38A illustrates an embodiment in which the implant 70 (shown with a cover present on the implant 70) is deployed within the patient's right atrium 1076 and then moved in the ventricular direction to couple the implant 70 to leaflets 1087 of the tricuspid valve 1083. The implant 70 may be deployed in the right atrium 1076 utilizing methods disclosed herein, including deploying the implant 70 from the capsule 106 into the right atrium 1076. The implant 70 may be moved in the ventricular direction in a variety of methods. In one embodiment, as shown in FIG. 38A, the implant 70 may be coupled to one or more tethers 2102. The tethers 2102 may be configured to be drawn away from the atrium 1076 to move the implant 70 in the ventricular direction to couple to the leaflets 1087. The tethers 2102 may be coupled to an anchor 2100 positioned on a wall of the right ventricle 1077 and may be configured to be withdrawn through the anchor 2100 to draw the tethers 2102 away from the atrium 1076. In other embodiments, other methods may be utilized to draw the tethers 2102 away from the atrium 1076. In one embodiment, a rail structure may be utilized to guide the implant 70 in the ventricular direction to couple to the valve leaflets 1087.

In one embodiment, the elongate shaft 12 may be utilized to push the implant 70 in the ventricular direction to couple to the valve leaflets 1087. In one embodiment, another push device (such as a device that may be passed through the superior vena cava 1081) may be utilized to push the implant 70 in the ventricular direction. A combination of methods may be utilized as desired. The implant 70 in position in the tricuspid annulus is shown in FIG. 38B.

In embodiments, the implants may include distal anchors for extending over and anchoring to heart valve flaps or leaflets 1087 as desired. The implant 70 may be anchored to the heart valve flaps or leaflet 1087. In embodiments, such distal anchors may be excluded.

The systems, apparatuses, and methods disclosed in regard to FIGS. 13A-38B and 62A-64C may be utilized with any embodiment disclosed in this application in combination or in substitution, or any other variation as desired.

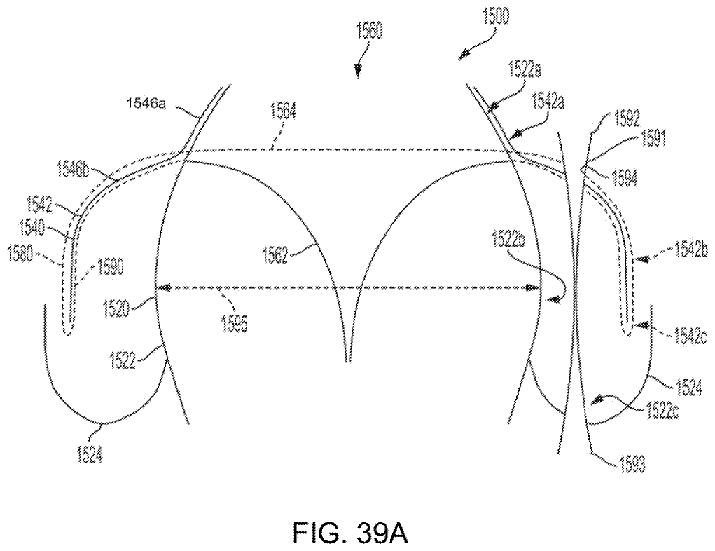

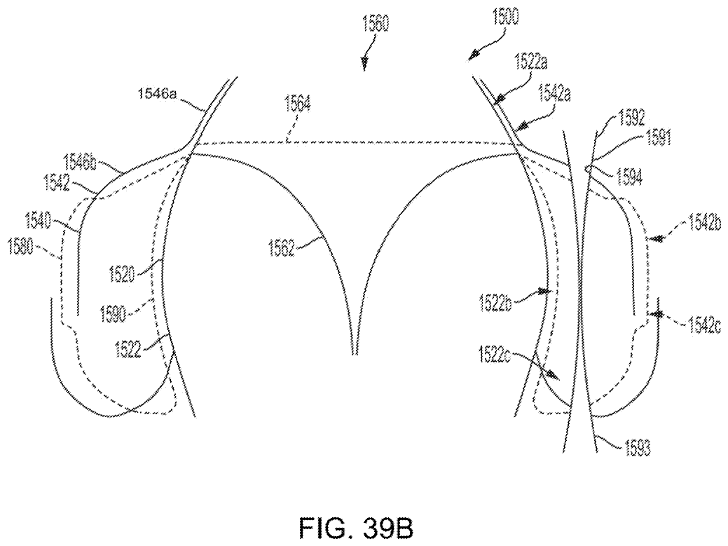

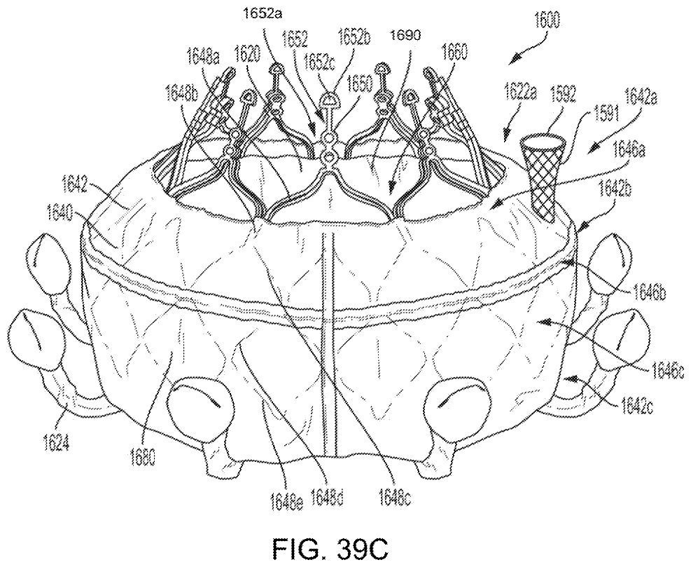

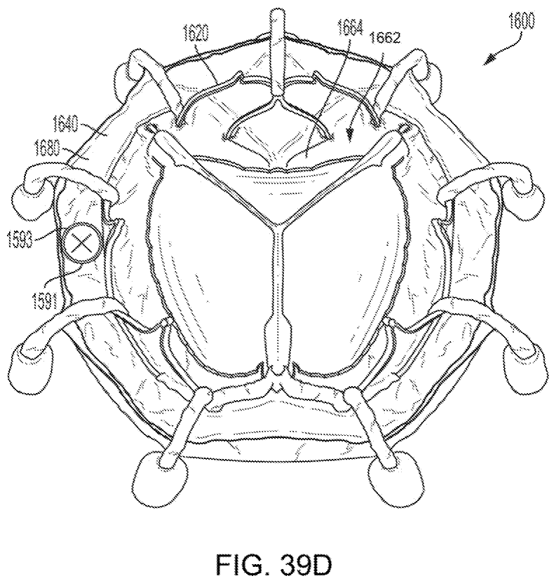

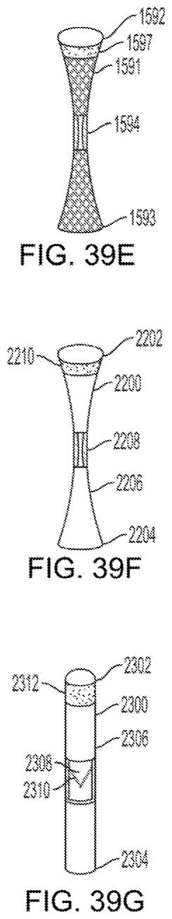

The implant to be utilized according to systems, apparatuses, and methods disclosed herein may include a port that may be configured to receive a diagnostic or therapeutic device. Such a diagnostic or therapeutic device may comprise a pacemaker pacing lead. Embodiments of such an implant are shown in FIGS. 39A-44.

Referring to FIG. 39A, an embodiment of an implant 1500 in an expanded configuration is illustrated. The implant 1500 can include an inner frame 1520, an outer frame 1540, a valve body 1560, and one or more skirts, such as an outer skirt 1580 and an inner skirt 1590.

With reference first to the inner frame 1520, the inner frame 1520 can include an inner frame body 1522 and an inner frame anchoring feature 1524. The inner frame body 1522 can have an upper region 1522a, an intermediate region 1522b, and a lower region 1522c. As shown, the inner frame body 1522 can have a generally bulbous shape such that the diameters of the upper region 1522a and the lower region 1522c are less than the diameter of the intermediate region 1522b.

While the illustrated inner frame body 1522 is bulbous, it is to be understood that the diameters of the upper region 1522a, the intermediate region 1522b, and/or the lower region 1522c can be the same such that the inner frame body 1522 is generally cylindrical along one or more regions. Moreover, all or a portion of the inner frame body 1522 can have a non-circular cross-section such as, but not limited to, a D-shape, an oval or an otherwise ovoid cross-sectional shape.

With reference next to the outer frame 1540 illustrated in FIG. 39A, the outer frame 1540 can be attached to the inner frame 1520 using any suitable fastener and/or other technique. Although the outer frame 1540 is illustrated as a separate component from the inner frame 1520, it is to be understood that the frames 1520, 1540 can be unitarily or monolithically formed.

As shown in the illustrated embodiment, the outer frame 1540 can include an outer frame body 1542. The outer frame body 1542 can have an upper region 1542a, an intermediate region 1542b, and a lower region 1542c.

The upper region 1542a of the outer frame body 1542 can include a first section 1546a and a second section 1546b. The first section 1546a can be sized and/or shaped to generally match the size and/or shape of the inner frame 1520.

The intermediate region 1542b of the outer frame body 1542 can extend generally downwardly from the outwardly-extending section 1546b of the upper region 1542a.

While the intermediate and lower regions 1542b, 1542c have been described as cylindrical, it is to be understood that the diameters of the upper end, the lower end, and/or the portion therebetween can be different. For example, all or a portion of the outer frame body 1542 can be have a non-circular cross-section such as, but not limited to, a D-shape, an oval or an otherwise ovoid cross-sectional shape.

The outer frame 1540, such as the outer frame body 1542 can be used to attach or secure the implant 1500 to a native valve, such as a native tricuspid valve. For example, the intermediate region 1542b of the outer frame body 1542 and/or the anchoring feature 1524 can be positioned to contact or engage a native valve annulus, tissue beyond the native valve annulus, native leaflets, and/or other tissue at or around the implantation location during one or more phases of the cardiac cycle, such as systole and/or diastole.

With continued reference to the implant 1500 illustrated in FIG. 39A, the valve body 1560 is attached to the inner frame 1520 within an interior of the inner frame body 1522. The valve body 1560 functions as a one-way valve to allow blood flow in a first direction through the valve body 1560 and inhibit blood flow in a second direction through the valve body 1560.

The valve body 1560 can include a plurality of valve leaflets 1562, for example three leaflets 1562, which are joined at commissures. The valve body 1560 can include one or more intermediate components 1564. The intermediate components 1564 can be positioned between a portion of, or the entirety of, the leaflets 1562 and the inner frame 1520 such that at least a portion of the leaflets 1562 are coupled to the frame 1520 via the intermediate component 1564.

With reference next to the outer skirt 1580 illustrated in FIG. 39A, a cover in the form of the outer skirt 1580 can be attached to the inner frame 1520 and/or outer frame 1540. As shown, the outer skirt 1580 can be positioned around and secured to a portion of, or the entirety of, the exterior of the outer frame 1540.

With reference next to the inner skirt 1590 illustrated in FIG. 39A, a cover in the form of the inner skirt 1590 can be attached to the valve body 1560 and the outer skirt 1580.

Although the implant 1500 has been described as including an inner frame 1520, an outer frame 1540, a valve body 1560, and skirts 1580, 1590, it is to be understood that the implant 1500 need not include all components.

The implant 1500 may include a port 1591 that is coupled to the valve body 1560 and is configured to receive a diagnostic or therapeutic device, which may comprise a pacemaker lead. The port 1591 as shown in FIG. 39A may include a tube that extends along the height of the implant 1500 to guide the pacemaker lead through the implant 1500. The tube of the port 1591 may include an entry opening 1592 and an exit opening 1593 with a central lumen 1594 extending between the entry opening 1592 and the exit opening 1593. The tube may have a cylindrical shape or may have a shape with opposed inverted funnels as shown in FIG. 39A. The tube may be configured to have a pacemaker lead passed from the entry opening 1592, along the central lumen 1594, and to the exit opening 1593.

The port 1591 may be positioned on the outer frame 1540 of the valve body 1560. The valve body 1560 may form a valve annulus 1595 that the valve leaflets 1562 are positioned in, and the port 1591 may be positioned outside of the valve annulus 1595. As such, the pacemaker lead passing through the port 1591 may avoid interference with the movement of the valve leaflets 1562.

The port 1591 may be configured to pass through the outer skirt 1580 of the implant 1500 and may pass within openings of the outer frame 1540 and between struts of the outer frame 1540. Other locations of the port 1591 may be utilized in other embodiments.

FIG. 39B illustrates an alternate embodiment of FIG. 39A with modifications to the design of the coverings, or skirts (or cloth) 1580/1590. As shown, the skirts 1580/1590 can contact both the inner frame 1520 and outer frame 1540. The skirts 1580/1590 can start on the inside of the outer 1540, transition to the outside of the outer frame 1540, then attach to the bottom of the outside of the inner frame 1520, then proceed up along the outside of the inner frame 1520. By closing the skirts 1580/1590, this could avoid/reduce clot formation/embolization.

The port 1591 accordingly may pass through both the upper portion of the skirt 1580 and the lower portion of the skirt 1580, as shown in FIG. 39B.

FIGS. 39C-D illustrate a perspective view of an embodiment of an implant 1600 in an expanded configuration. The implant 1600 may be similar in construction to the implant 1500 described above. The implant 1600 can include an inner frame 1620, an outer frame 1640, a valve body 1660, and one or more skirts, such as an outer skirt 1680 and an inner skirt 1690. A perspective view of the port 1591 is shown extending from an upper surface of the implant 1600.

With reference first to the outer frame 1640 illustrated in FIGS. 39C—D, the outer frame 1640 can be attached to the inner frame 1620 using any known fasteners and/or techniques. Although the outer frame 1640 is illustrated as a separate component from the inner frame 1620, it is to be understood that the frames 1620, 1640 can be unitarily or monolithically formed.

As shown in the illustrated embodiment, the outer frame 1640 can include an outer frame body 1642. The outer frame body 1642 can have an upper region 1642a, an intermediate region 1642b, and a lower region 1642c. At least a portion of the upper region 1642a of the outer frame body 1642 can be sized and/or shaped to generally match the size and/or shape of an upper region 1622a of the inner frame 1620.

When in an expanded configuration such as in a fully expanded configuration, the outer frame body 1642 can have a shape similar to that of outer frame body 1542 described above in connection with FIG. 39A. However, it is to be understood that all or a portion of the outer frame body 1642 can be have a non-circular cross-section such as, but not limited to, a D-shape, an oval or an otherwise ovoid cross-sectional shape.

With continued reference to the implant 1600 illustrated in FIG. 39C, the outer frame body 1642 can include a plurality of struts with at least some of the struts forming cells 1646a-c.

The upper row of cells 1646a can have an irregular octagonal shape such as a “heart” shape. This additional space can beneficially allow the outer frame 1640 to retain a smaller profile when crimped. The cell 1646a can be formed via a combination of struts. As shown in the illustrated embodiment, the upper portion of cells 1646a can be formed from a set of circumferentially-expansible struts 1648a having a zig-zag or undulating shape forming a repeating “V” shape.

The middle portion of cells 1646a can be formed from a set of struts 1648b extending downwardly from bottom ends of each of the “V” shapes.

The lower portion of cells 1646a can be formed from a set of circumferentially-expansible struts 1648c having a zig-zag or undulating shape forming a repeating “V” shape.

The middle and/or lower rows of cells 1646b-c can have a different shape from the cells 1646a of the first row. The middle row of cells 1646b and the lower row of cells 1646c can have a diamond or generally diamond shape. The diamond or generally diamond shape can be formed via a combination of struts.

The upper portion of cells 1646a can be formed from the set of circumferentially-expansible struts 1648c such that cells 1646b share struts with cells 1646a. The lower portion of cells 1646b can be formed from a set of circumferentially-expansible struts 1648d. As shown in the illustrated embodiment, one or more of the circumferentially-expansible struts 1648d can extend generally in a downward direction generally parallel to the longitudinal axis of the outer frame 1640.

The upper portion of cells 1646c can be formed from the set of circumferentially-expansible struts 1648d such that cells 1646c share struts with cells 1646b. The lower portion of cells 1646c can be formed from a set of circumferentially-expansible struts 1648e. Circumferentially-expansible struts 1648e can extend generally in a downward direction.

As shown in the illustrated embodiment, the implant 1600 can include a set of eyelets 1650. The upper set of eyelets 1650 can extend from an upper region 1642a of the outer frame body 1642. As shown, the upper set of eyelets 1650 can extend from an upper portion of cells 1646a, such as the upper apices of cells 1646a. The upper set of eyelets 1650 can be used to attach the outer frame 1640 to the inner frame 1620.

The outer frame 1640 can include a set of locking tabs 1652 extending from at or proximate an upper end of the upper region 1642a. As shown, the locking tabs 1652 can extend upwardly from the set of eyelets 1650. The outer frame 1640 can include twelve locking tabs 1652, however, it is to be understood that a greater number or lesser number of locking tabs can be used. The locking tabs 1652 can include a longitudinally-extending strut 1652a. At an upper end of the strut, the locking tab 1652 can include an enlarged head 1652b. As shown, the enlarged head 1652b can have a semi-circular or semi-elliptical shape forming a “mushroom” shape with the longitudinal strut 1652a. The locking tab 1652 can include an eyelet 1652c which can be positioned through the enlarged head 1652b. It is to be understood that the locking tab 1652 can include an eyelet at other locations, or can include more than a single eyelet.

The locking tab 1652 can be advantageously used with multiple types of delivery systems. For example, the shape of the struts and the enlarged head 1652b can be used to secure the outer frame 1640 to a “slot” based delivery system, such as the inner retention member 40 described above. The eyelets 1652c and/or eyelets 1650 can be used to secure the outer frame 1640 to a “tether” based delivery system such as those which utilize sutures, wires, or fingers to control delivery of the outer frame 1640 and the implant 1600. This can advantageously facilitate recapture and repositioning of the outer frame 1640 and the implant 1600 in situ.

The outer frame 1640, such as the outer frame body 1642 can be used to attach or secure the implant 1600 to a native valve, such as a native tricuspid valve. For example, the intermediate region 1642b of the outer frame body 1642 and/or the anchoring feature 1624 can be positioned to contact or engage a native valve annulus, tissue beyond the native valve annulus, native leaflets, and/or other tissue at or around the implantation location during one or more phases of the cardiac cycle, such as systole and/or diastole. As another example, the outer frame body 1642 can be sized and positioned relative to the inner frame anchoring feature 1624 such that tissue of the body cavity positioned between the outer frame body 1642 and the inner frame anchoring feature 1624, such as native valve leaflets and/or a native valve annulus, can be engaged or pinched to further secure the implant 1600 to the tissue. As shown, the inner frame anchoring feature 1624 includes nine anchors; however, it is to be understood that a fewer or greater number of anchors can be used. In some embodiments, the number of individual anchors can be chosen as a multiple of the number of commissures for the valve body 1660.

The valve body 1660 can include a plurality of valve leaflets 1662, for example three leaflets 1662, which are joined at commissures. The valve body 1660 can include one or more intermediate components 1664.

With reference next to the outer skirt 1680 illustrated in FIG. 39C, the covering or outer skirt 1680 can be attached to the inner frame 1620 and/or outer frame 1640. The outer skirt 1680 can be positioned around and secured to a portion of, or the entirety of, the exterior of the outer frame 1640. The inner skirt 1690 can be attached to the valve body 1660 and the outer skirt 1680.

Although the implant 1600 has been described as including an inner frame 1620, an outer frame 1640, a valve body 1660, and skirts 1680, 1690, it is to be understood that the implant 1600 need not include all components. For example, in some embodiments, the implant 1600 can include the inner frame 1620, the outer frame 1640, and the valve body 1660 while omitting the skirt 1680. Moreover, although the components of the implant 1600 have been described and illustrated as separate components, it is to be understood that one or more components of the implant 1600 can be integrally or monolithically formed. For example, in some embodiments, the inner frame 1620 and the outer frame 1640 can be integrally or monolithically formed as a single component.

Referring to FIG. 39C, the upper end of the port 1591 is shown extending from an upper surface of the skirt 1680 and the outer frame 1640. The opening 1592 may be raised above the upper surface of the skirt 1680 and the outer frame 1640 to allow a user to pass a diagnostic or therapeutic device, which may comprise a pacemaker pacing lead through the opening 1592.

FIG. 39D shows a bottom view of the implant 1600, illustrating the position of the exit opening 1593.

Referring to FIGS. 39E—G, the port may have a variety of forms. The ports are shown isolated from the implants. Referring to FIG. 39E, the body of the tube of the port 1591 may be made of a woven or textile material that exhibits bias to contract the central lumen 1594. The contraction of the central lumen 1594 may form a seal against the diagnostic or therapeutic device, which may comprise a pacemaker pacing lead, as it is inserted through the port 1591, to prevent reverse blood flow through the port 1591. The woven material may be made of wires such as nitinol wires that are interwoven and heat set. Other materials may be utilized as desired.

The port 1591 may include a location marker such as a radiopaque marker 1597 that identifies the location of the port 1591 and particularly the opening 1592 of the port 1591 under imaging.

FIG. 39F illustrates an embodiment of the port 2200 in which the port 2200 includes a tube having an entry opening 2202, an exit opening 2204 and a body 2206 positioned between the entry opening 2202 and the exit opening 2204. The body 2206 may surround a central lumen 2208 that the diagnostic or therapeutic device, which may comprise a pacemaker pacing lead, passes through. The body 2260 may be made of a polymer such as an elastomeric material such as a fluoroelastomer or a silicone, configured to be biased towards the central lumen 2208. The bias of the body 2260 towards the central lumen 2208 may form a seal against the pacemaker pacing lead as it is inserted through the port 2200, to prevent reverse blood flow through the port 2200. Other materials may be utilized as desired.

The port 2200 may include a location marker such as a radiopaque marker 2210 that identifies the location of the port 2200 and particularly the opening 2202 of the port 2200 under imaging.

FIG. 39G illustrates an embodiment of the port 2300 in which the port 2300 includes a tube having an entry opening 2302, an exit opening 2304 and a body 2306 positioned between the entry opening 2302 and the exit opening 2304. The body 2306 may surround a central lumen 2308 that the pacemaker pacing lead passes through. The central lumen 2308 may include a valve 2310 positioned therein, that may form a seal against the diagnostic or therapeutic device, which may comprise a pacemaker pacing lead, as it is inserted through the port 2300, to prevent reverse blood flow through the port 2300. The valve 2310 may comprise a duckbill valve or other form of valve.

The port 2300 may include a location marker such as a radiopaque marker 2312 that identifies the location of the port 2300 and particularly the opening 2302 of the port 2300 under imaging. The body 2306 may be made of a polymer, an elastomer, silicone, or a textile material. Other materials may be utilized as desired.

Any embodiment of port disclosed herein may be impregnated on either an outside surface or inside surface, or both, of a drug coating for release into the patient's body. Further, a coating may be provided on either an outside surface or inside surface, or both, to provide the surface with hydrophilic or hydrophobic properties, or antithrombic properties.

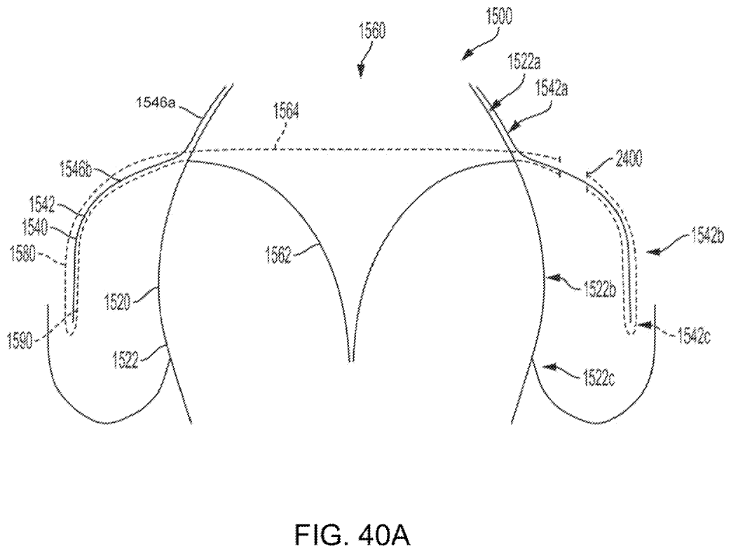

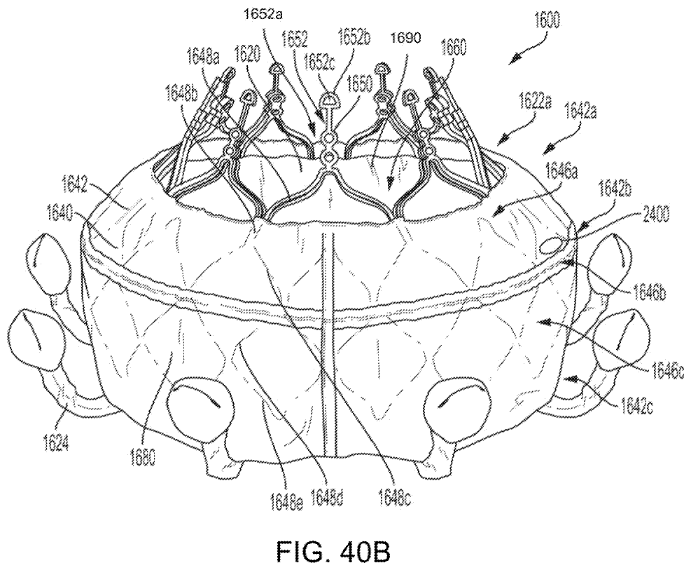

FIGS. 40A—B illustrate an embodiment of a port 2400 in which the port 2400 comprises an opening in the valve body 1560 of the implant 1500. The port 2400 may extend through a cover or skirt of the implant 1500, which may include an outer skirt 1580 as shown in FIG. 40A. The opening may be made of a material that is biased towards the center of the opening, such that as a pacemaker pacing lead is passed through the opening, the material may form a seal against the pacemaker pacing lead as it is inserted through the port 2400, to prevent reverse blood flow through the port 2400. For example, an elastic material may form a seal against the pacing lead. As shown in FIG. 40B, the opening may be positioned between struts of the outer frame to allow the lead to have a path to pass through. The opening may be surrounded by a location marker such as a radiopaque marker that identifies the location of the port 2400 and particularly the opening of the port 2400 under imaging.

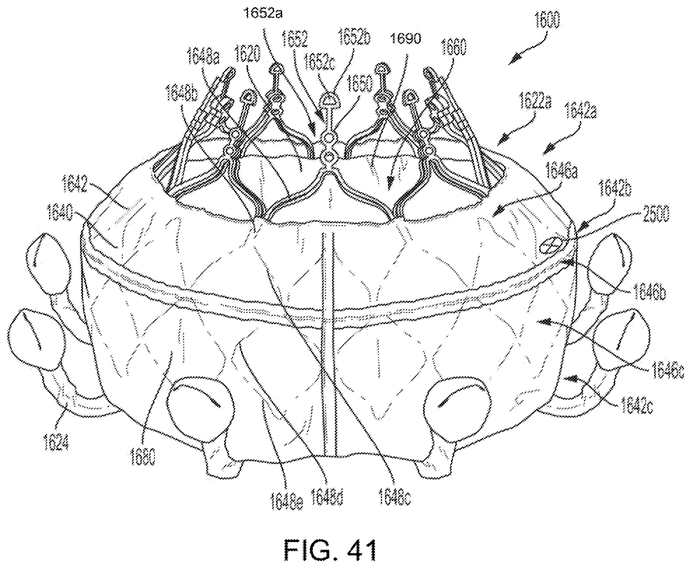

FIG. 41 illustrates an embodiment in which the port 2500 comprises a tearable portion of the valve body 1660. The tearable portion may be a tearable portion of the outer skirt 1680 as shown in FIG. 41. The tearable portion may pass a diagnostic or therapeutic device therethrough. The tearable portion may be configured to be penetrated by a puncture device or the pacemaker pacing lead to pass through the tearable portion. The material surrounding the resulting opening in the skirt 1680 may be configured to be biased towards the opening, to prevent reverse blood flow through the port 2500. The tearable portion may form flaps that press against the pacing lead, to seal against the pacing lead as a reverse flow is applied to the flaps against the lead. An elastomer material may be used to form a seal against the lead. As shown in FIG. 41, the opening may be positioned between struts of the outer frame, to allow a path for the pacing lead. The opening may be surrounded by a location marker such as a radiopaque marker that identifies the location of the port 2500 and particularly the opening of the port 2500 under imaging.

In one embodiment, a port may be positioned outside of the outer valve body, for positioning between the outer valve body and the annulus of the heart valve. The port may comprise a loop of material or the like that the diagnostic or therapeutic device is passed through.

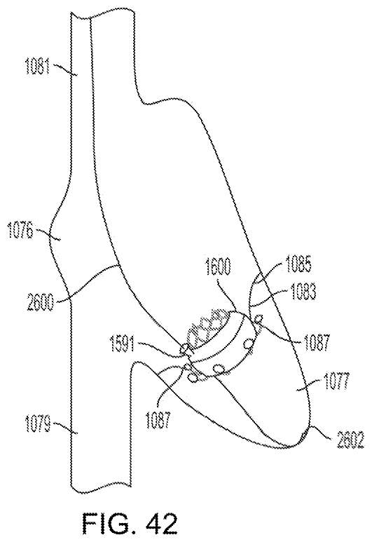

FIG. 42 illustrates use of the port 1591 as shown in FIG. 39C. The diagnostic or therapeutic device, which may comprise a pacemaker pacing lead 2600 may be passed through the port 1591. The tip 2602 of the pacemaker pacing lead 2600 may be positioned within the right ventricle.

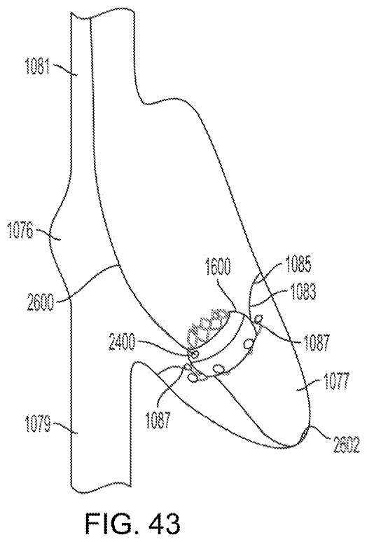

FIG. 43 illustrates use of the port 2400 as shown in FIG. 40B. The diagnostic or therapeutic device, which may comprise a pacemaker pacing lead 2600 may be passed through the port 2400. The tip 2602 of the pacemaker pacing lead 2600 may be positioned within the right ventricle.

A method may include passing a diagnostic or therapeutic device through a port positioned on a prosthetic heart valve body. The prosthetic heart valve body may form a prosthetic heart valve annulus. The port may include a tube for the diagnostic or therapeutic device to be passed through.

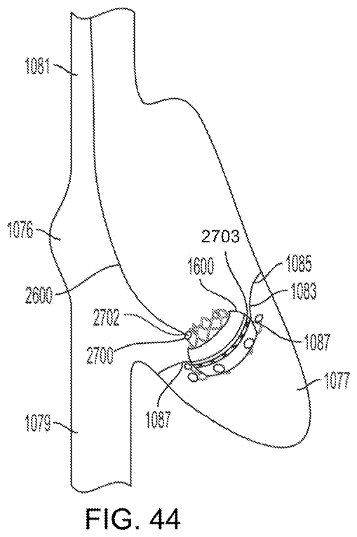

FIG. 44 illustrates an embodiment in which the port 2700 is configured to couple to the pacemaker pacing lead 2600 to form an electrical connection between the implant 1600 and the pacemaker pacing lead 2600. The tip 2702 of the pacemaker pacing lead 2600 may be configured to couple to the port 2700 and provide electrical energy to the implant 1600. The frame of the implant 1600 may provide electrical energy to pace functioning of the patient's heart. The pacemaker pacing lead 2600 may couple directly to the implant frame, as shown in FIG. 44. The implant frame may be made of nitinol or another electrically conductive material. The implant may include one or more electrical terminals 2703 that are in contact with the patient's body, and may provide the electrical energy to the patient's body to pace functioning of the patient's heart. The terminals, for example, may be on the outside of the body of the implant, or may be positioned on the valve leaflet anchors or other part of the implant in contact with a portion of the patient's heart.

In embodiments disclosed herein, the prosthetic valve body may be deployed to the patient's heart valve annulus utilizing methods disclosed herein. The valve body may be expanded within the heart valve annulus, and may be anchored to the heart valve flaps or leaflets of the heart valve. The valve body may be contacted to the patient's heart valve.

A method may include coupling a pacemaker pacing lead to a prosthetic heart valve body positioned within a patient's heart valve annulus to provide electrical energy through the pacemaker pacing lead and through the prosthetic heart valve body to pace functioning of the patient's heart. The method may include providing electrical energy through the frame. The prosthetic valve body may include one or more electrical terminals in contact with a portion of the patient's heart. Electrical energy may be provided through the pacemaker pacing lead and through the prosthetic heart valve body to pace functioning of the patient's heart.

Any embodiments of ports for a pacemaker pacing lead may be utilized acutely, if conduction disturbance is detected at the time of implant, or chronically, if conduction issues develop over time.

The diagnostic or therapeutic device may not only comprise a pacemaker pacing lead, but may comprise other forms of devices, such as catheters or other medical devices to be passed through the implant.

The embodiments of implants disclosed herein may be utilized solely, or across embodiments as desired. Such embodiments may be utilized in a tricuspid or mitral valve, or other valve as desired. Features of the embodiments of implants may be combined across embodiments as desired.

Any and all of the embodiments disclosed herein may be utilized with motorized implant delivery systems. Further, in any and all embodiments, the delivery system may utilize a processor for control of at least one motor for actuating a delivery apparatus. Further, in any and all embodiments, the delivery system may include sensors as disclosed herein. The delivery system may include sensors configured sense one or more of a condition of the patient's body or a condition of the delivery apparatus. The processor may process the signals provided by the sensors, which may comprise feedback signals to the processor.

Features of such systems are disclosed in U.S. Provisional Patent Application No. 62/837,641, filed Apr. 23, 2019, the entire contents of which are incorporated herein by reference. Features of such systems are also disclosed in PCT Application No. PCT/US2020/029138, filed Apr. 21, 2020, the entire contents of which (along with the U.S. National Stage application for PCT Application No. PCT/US2020/029138) are incorporated herein by reference.

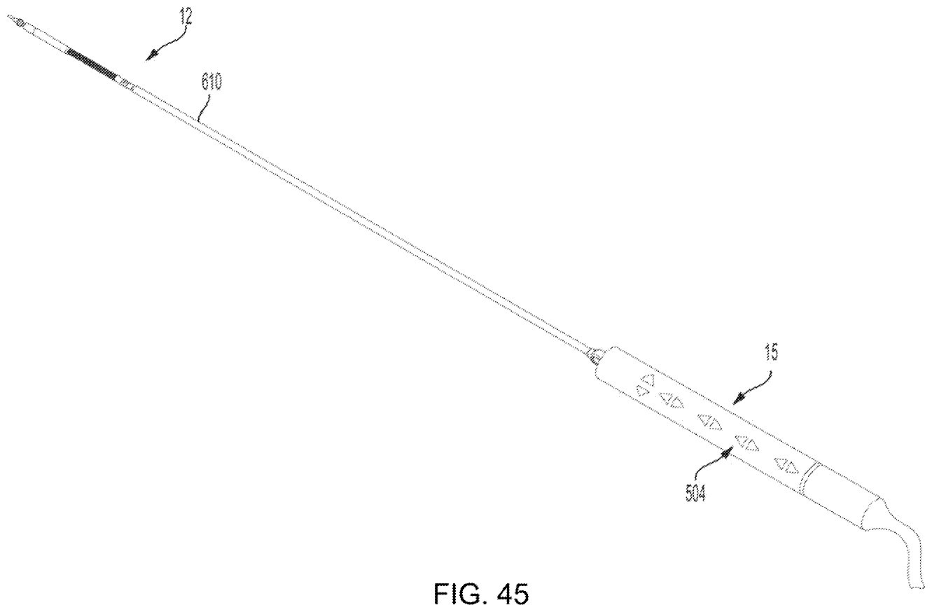

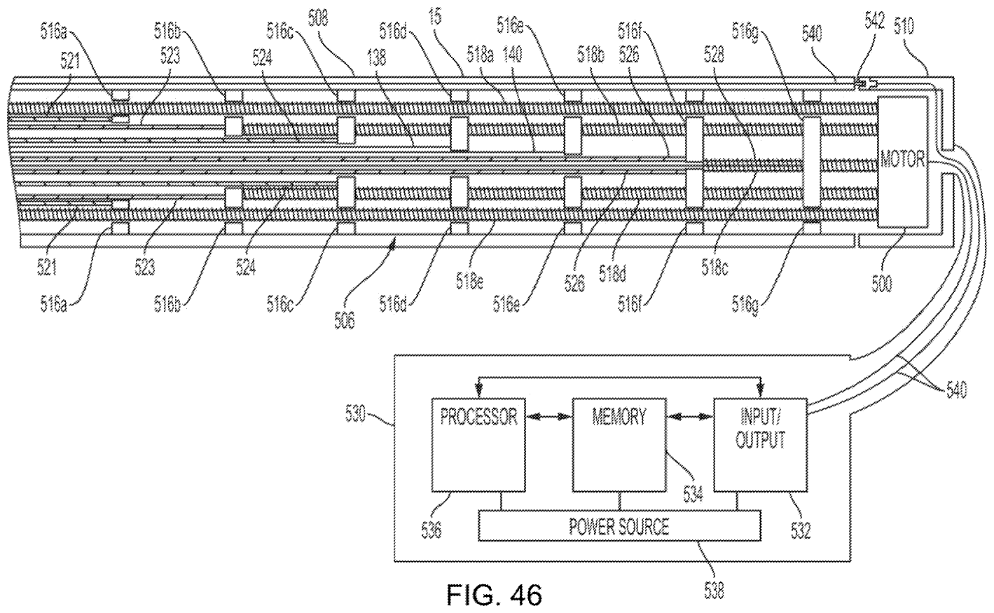

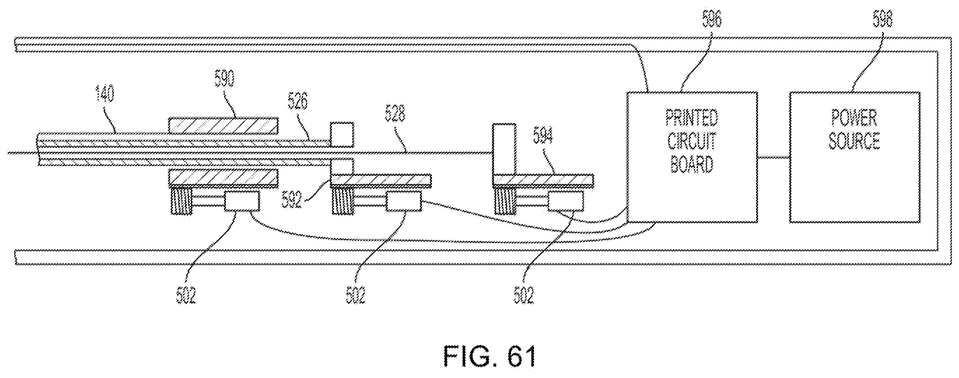

Referring to FIG. 45, the elongate shaft 12 and housing in the form of a handle 15 may form a delivery apparatus that is configured to deliver the implant 70 to a location within a patient's body. The delivery apparatus may also include the deflection mechanisms disclosed herein, which may include use of the sheath 610 as shown in FIG. 45. The delivery system 10 may include at least one motor that is configured to actuate at least a portion of the delivery apparatus. The actuation of at least a portion of the delivery apparatus may include deflection of a portion of the delivery apparatus (including the elongate shaft) or other movement of the delivery apparatus, and may include actuation of an operation of the delivery apparatus. The operation may include deployment (whether full or partial) of the implant 70 to the body location, among other operations of the delivery apparatus. The motor may comprise a motor 500 as shown in FIG. 46, or may comprise a plurality of motors 502 shown in FIG. 61 (i.e., at least one motor), among other forms of motors.

As shown in FIG. 45, the housing in the form of the handle 15 may be positioned at the proximal end 11 of the elongate shaft 12. The proximal end 11 of the elongate shaft 12 may be coupled to the handle 15. The handle 15 may include a control device 504 configured to control the at least one motor. The control device 504 as shown in FIG. 45 may include a plurality of buttons; however, in other embodiments other forms of control devices may be utilized. The control device 504 may be positioned on the handle 15 as shown in FIG. 45 or may be located remotely.

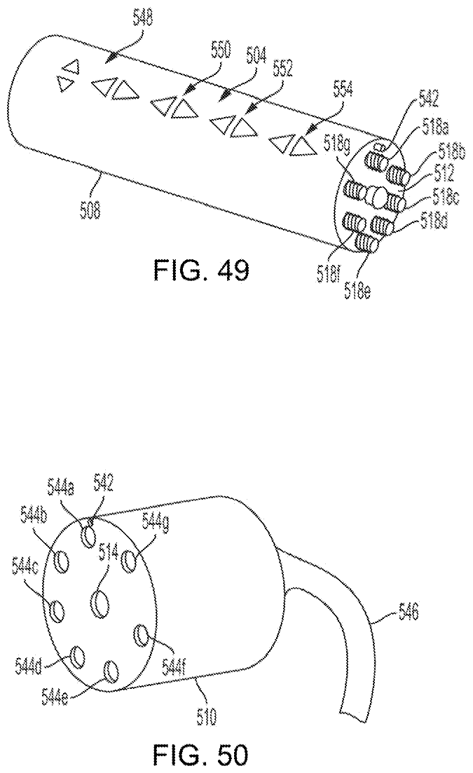

FIG. 46 illustrates a cross section of the handle 15 including the motor 500 and an actuation mechanism 506 that may be utilized to actuate at least a portion of the delivery apparatus. In various embodiments, the motor and actuation mechanism may be used to actuate pull wires during advancement through the vasculature. The motor and actuation mechanism may be used to actuate to actuate shafts/sheaths for deploying and releasing the implant at the treatment site. The body of the handle 15 may include multiple parts, including a distal portion 508 and a proximal portion 510. The distal portion 508 as shown in FIG. 46 may be configured to retain the actuation mechanism 506 and the proximal portion 510 may be configured to retain the motor 500. In other embodiments, other components may be positioned in respective distal 508 and proximal portions 510, and in certain embodiments the handle 15 may include a single body. In the embodiment shown in FIG. 46, the distal portion 508 and proximal portion 510 may be configured to couple together via a coupler 512, 514 (marked in FIGS. 49 and 50), and may be separable from each other in certain embodiments.

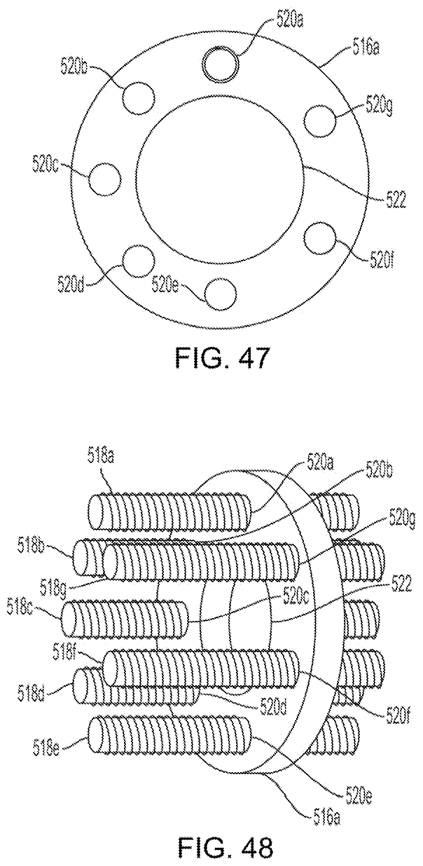

The actuation mechanism 506 may take the form as shown in FIG. 46 and may include a plurality of adaptors 516a-g configured to engage with a plurality of drive rods 518a-g (drive rods 518f-g are marked in FIG. 48). Each adaptor 516a-g may comprise a plate or other body including a plurality of apertures. FIG. 47 illustrates a front plan view of the adaptor 516a. The adaptor 516a as shown in FIG. 47 may include apertures 520a-g and 522. The apertures 520a-g may each be configured to allow a respective drive rod 518a-g to pass therethrough (as represented in FIG. 48). The apertures 520b-g may each be configured to be smooth bearing surfaces, that do not engage the respective drive rods 518b-g. The aperture 520a, however, may be configured with a threaded surface or other surface that engages the drive rod 518a. For example, the drive rod 518a may include a gear threading and the aperture 520a may include a threading that matches the gear threading. Such a configuration allows the drive rod 518a to actuate the adaptor 516a in two directions (distal and proximal) based on the direction that the drive rod 518a is rotating. In other embodiments, other forms of engagement may be utilized.

The central aperture 522 may allow other components of the actuation mechanism 506 such as assembly connectors to pass through the central aperture to couple to the remaining respective adaptors 516a-g.

FIG. 48 illustrates a perspective view of adaptor 516a with representative drive rods 518a-g extending through the apertures 520a-g.

The other adaptors 516b-g may be configured similarly as the adaptor 516a, however, each respective adaptor 516b-g may have an aperture that is configured to engage the respective drive rods 518b-g, with the remaining apertures comprising smooth bearing surfaces. For example, for adaptor 516b, the equivalent aperture to aperture 520b may be configured to engage drive rod 518b while the remaining equivalent apertures to apertures 520a, c-g may comprise smooth bearing surfaces. Adaptors 516c-g have similar respective apertures configured to engage respective drive rods 518c-g. In this manner, a single drive rod 518a-g may be configured to actuate a respective dedicated adaptor 516a-g. The remaining drive rods may pass through the remaining adaptors without engaging the adaptor.

Referring to FIG. 46, the adaptors 516a-g may be configured to slide within the interior cavity of the housing comprising the handle 15. The outer surfaces of the adaptors 516a-g for example, may be positioned on a track within the handle 15 or otherwise configured to slide or move within the handle 15.

The drive rods 518a-g may extend longitudinally along the interior of the handle 15 and may be configured to engage a respective adaptor 516a-g. For example, FIG. 46 illustrates the adaptor 516a engaged by drive rod 518a and the adaptor 516g engaged by drive rod 518e (in a configuration in which adaptor 516g was configured to be engaged by drive rod 518e, other configurations, e.g., the adaptor 516g being engaged by drive rod 518g, may be utilized). Proximal ends of the drive rods 518a-g may be configured to engage and be actuated by motor 500.

The adaptors 516a-g may be coupled to assembly connectors that couple to respective portions of the assemblies (the outer sheath assembly 22, the mid shaft assembly 21, the rail assembly 20, the inner assembly 18, and the nose cone assembly 31) including the pull wire assemblies 138, 140. In certain embodiments, the adaptors 516a-g may couple to particular components comprising each of the assemblies, for example, the adaptor 516a may couple directly to the nose cone shaft 27 in certain embodiments. The coupling of the adaptors 516a-g to the assembly connectors may be such that the adaptor 516a couples to an assembly connector 521 for the outer sheath assembly 22. The adaptor 516b may couple to an assembly connector 523 for the mid shaft assembly 21. The adaptor 516c may couple to an assembly connector 524 for the rail assembly 20. The adaptor 516d may couple to an assembly connector for the distal pull wires 138 or may couple to the distal pull wires 138 directly. The adaptor 516e may couple to an assembly connector for the proximal pull wires 140 or may couple to the proximal pull wires 140 directly. The adaptor 516f may couple to an assembly connector 526 for the inner assembly 18. The adaptor 516g may couple to an assembly connector 528 for the nose cone assembly 31. The assembly connectors 521, 523, 524, 526, 528 may comprise sheaths that extend concentricly over each other, or may comprise rods, wires, or other forms of connectors. The assembly connectors 521, 523, 524, 526, 528 may be configured to pass through the central aperture of the respective adaptors 516a-g (for example aperture 522 shown in FIG. 47).

The assembly connectors 521, 523, 524, 526, 528 may have a proximal portion coupled to the respective adaptor 516a, b, c, f, g and a distal portion coupled to a portion of the respective assembly in order to actuate the respective assembly. For example, the assembly connector 521 may couple to the outer sheath assembly 22 such that movement of the assembly connector 521 moves the outer covering, or sheath of the outer sheath assembly 22 to expose the implant 70 in the capsule 106. The assembly connector 523 may couple to the mid shaft assembly 21 such that movement of the assembly connector 523 moves the outer retention member 42. The assembly connector 524 may couple to the rail assembly 20 such that movement of the assembly connector 524 moves the rail assembly 20. The movement of the adaptors 516d and 516e may move the respective pull wires 138, 140. The assembly connector 526 may couple to the inner assembly 18 such that movement of the assembly connector 526 moves the inner retention member 40. The assembly connector 528 may couple to the nose cone assembly 31 such that movement of the assembly connector 528 moves the nose cone 28. The respective drive rod 518a-g may thus be actuated by the motor 500 to selectively move a respective adaptor 516a-g and accordingly a respective portion of the assemblies (the outer sheath assembly 22, the mid shaft assembly 21, the rail assembly 20, the inner assembly 18, and the nose cone assembly 31).

The motion of the assemblies (the outer sheath assembly 22, the mid shaft assembly 21, the rail assembly 20, the inner assembly 18, and the nose cone assembly 31) may be a translation of the respective assemblies, which may include the pull wires 138, 140, to produce the desired movement (e.g., deflection) or operation (e.g., deployment of the implant). For example, the motor 500 may be configured to translate a rail shaft of the rail assembly 20 relative to an inner sheath of the inner assembly 18 and the outer sheath of the outer sheath assembly 22. The motor 500 may be configured to translate the outer sheath of the outer sheath assembly 22 relative to the inner sheath of the inner assembly 18 in certain embodiments. The motor 500 may be configured to translate any of the assemblies relative to each other to produce a desired result. The motor 500 may be configured to steer the rail assembly 20, for example, by actuating the pull wires 138, 140. Other movements may include actuating a depth of the elongate shaft 12, and actuating an operation of the elongate shaft 12, for example a full or partial deployment of the implant 70. The movement may be of a deflection mechanism disclosed herein.

In other embodiments, the actuation of the delivery apparatus with the motor 500 may occur in a different manner than shown in FIG. 46. In one embodiment the configuration of the actuation mechanism 506 may differ from the configuration shown in FIG. 46.

The delivery system 10 may include a controller 530 that is configured to control operation of the motor 500 and thus control actuation of the portion of the delivery apparatus. The controller 530 as shown in FIG. 46 may include an input device and an output device (marked as item 532). The controller 530 may include a memory 534 and a processor 536. The controller may include a power source 538.

The input device and output device 532 may have a plurality of configurations, including electrical ports or terminals that are configured to transmit electrical signals. The input device may be configured to receive signals from the motor 500 as well as from sensors positioned on the delivery system 10. The output device may be configured to transmit signals to the motor 500 or other components of the system 10 which may be received from the processor 536 or other components of the system 10. In certain embodiments, the input device and output device 532 may comprise wireless transmission devices, such as a Wi-Fi or Bluetooth device or other device configured for wireless communication. In an embodiment in which the controller 530 is positioned remotely from the delivery apparatus, the input device and output device 532 may be configured to transmit and receive information via the Internet or other form of communication medium. In other embodiments, other forms of input devices and output devices may be utilized.

The memory 534 may be configured to store programs for operation by the processor 536 as well as other data desired to be stored in the controller 530. The memory 534 may be configured to store and log data regarding the patient and the operation of the delivery apparatus and the motor 500 during a procedure, thereby allowing the system to learn from past events. The learning aspect may be based on an algorithm capable of identifying procedures that have produced positive outcomes in the past, thereby allowing the system to continually refine the procedure to enhance the probability of success. Preferably, data could be pooled from different patients, different clinicians and/or different hospitals. The compilation of data could be used to increase precision and improve outcomes in future procedures. This could be achieved, for example, by comparing characteristics of a new patient with patients who have been treated in the past. Data from procedures on past patients with similar anatomies and/or other parameters, such as the patient's gender, age, and health, would be particularly useful. Other parameters could be incorporated into the algorithm, such as the clinician's skill level and amount of experience and/or the facilities available at the hospital. The data may be used in a machine learning algorithm utilizing data from past implantation procedures or from characteristics of the patient.

The memory 534 may comprise various forms of memory including a hard disk, solid state memory, various forms of RAM or ROM, or other forms of memory. In one embodiment, the memory 534 may be configured to be removable from the controller 530 for storage and/or data analysis. Separate memory 534 may be installed into the controller 530 or swapped into or out of the controller 530 as desired for particular forms of operation.

The processor 536 may be configured to perform processes disclosed herein and may be configured to provide signals to components of the system 10 for example, the motor 500 to perform desired processes. The processor 536 may be configured to operate the motor 500, or at least one motor 500, to actuate at least a portion of the delivery apparatus. The processor 536 may be configured to operate at least one motor 500 to move a portion of the delivery apparatus (e.g., deflect or control a depth of the elongate shaft 12), or perform an operation of the delivery apparatus, which may include deploying the implant 70 from the delivery apparatus. The processor 536 may be configured to execute processes stored in the memory 534. The processor 536 may be configured to receive signals from components of the system 10 such as a control device (for example control device 504) or sensors of the system 10. The processor 536 may be configured to process and perform operations based on those signals. The processor 536 may comprise a microprocessor, or other form of processor as desired. In one embodiment, the processor 536 may comprise a plurality of processors, and in one embodiment may be distributed in a cloud computing environment or the like.

The power source 538 may be configured to provide power to the components of the controller 530, and may be configured to provide power to the motor 500 or other components of the system 10. The power source 538 may comprise one or more batteries according to certain embodiments, which may be rechargeable and detachable from the controller 530 or other components of the system 10 as desired. In one embodiment, the power source 538 may comprise a power plug such as an AC plug, and may include a power regulator for converting the AC power to a power usable by the system 10. Other forms of power sources 538 (e.g., super capacitors, solar cells, among others) may be used in other embodiments as desired.

The components of the controller 530 may be positioned together as shown in FIG. 46 or may be distributed as desired. The components of the controller 530 may be positioned in a separate housing, or control box, and may be coupled to the delivery apparatus with a cable or the like. FIG. 46 illustrates a cabled connection of the controller 530 to the delivery apparatus. In other embodiments, wireless communication may be possible between one or more components of the controller 530 and the delivery apparatus. In other embodiments, components of the controller 530 may be positioned within the housing of the delivery apparatus, for example, in a configuration shown in FIG. 61.

Power and signal connectors 540 may extend between the controller 530 and the delivery apparatus. For example, a signal connector 540 is shown extending along a portion of the handle 15 and may couple between the distal portion 508 of the handle 15 and the proximal portion 510 at the electrical coupler 542. Power connectors 540 may extend to the motor 500 from the power source 538 of the controller 530.

FIG. 49 illustrates a perspective view of the distal portion 508 of the handle 15. The distal portion 508 of the handle 15 may be configured to separate from the proximal portion 510 (shown in FIG. 50). Such a configuration may allow a particular portion of the handle 15 of the delivery apparatus to be utilized in delivery of an implant, and then separated from another portion (e.g., proximal portion 510) of the handle 15 such that sterilization or discard of the distal portion 508 may occur. This process may separate electrical components of the system 10, which may include the motor 500 positioned within the proximal portion 510, or may include the controller 530, from components that are inserted into or contact portions of the patient's body. This may enhance reusability of the system 10 and reduce the overall complexity associated with sterilizing the system 10. As shown in FIG. 49, proximal portions of the drive rods 518a-g may extend proximally from the distal portion 508 of the handle 15, for coupling to respective apertures 544a— g in the proximal portion 510 of the handle 15. The proximal portions of the drive rods 518a-g may couple to the respective apertures 544a-g to allow the motor 500 to engage the drive rods 518a-g. The electrical coupler 542 and coupler 512 are also shown protruding from the distal portion 508 of the handle 15.

FIG. 50 illustrates a perspective view of the proximal portion 510 of the handle 15. The proximal portion 510 may include a cable 546 or other connector that couples the proximal portion 510 to the controller 530, which may be contained in a control box or the like.

Referring again to FIG. 49, the control device 504 is shown on the distal portion 508 of the handle 15 as including a plurality of buttons. The control device 504 may be configured to receive an input from a user to operate the motor 500 and thus actuate a portion of the delivery apparatus. The control device 504 may be configured to send a signal directly to the motor 500 or may be sent to the processor 536 of the controller 530 for processing. The control device 504 may be configured to control deflection and movement of the delivery apparatus. The control device 504 may be configured to control an operation of the delivery apparatus such as deployment of the implant 70. The control device 504 may have a variety of forms, and as shown in FIG. 49 may have portions designated to control certain movements or operations of the delivery apparatus.

The control device 504 of FIG. 49 may include buttons 548 that control the rail assembly 20 and particularly the direction of deflection of the rail assembly 20, which may be in at least two planes. The buttons 548 may be configured to control steering of the rail assembly 20. The user may press the desired button 548 to cause the motor to actuate the delivery apparatus to deflect in the desired direction. The control device 504 of FIG. 49 may include buttons 550 that control the depth of the elongate shaft 12, for example, by sliding the assemblies including the outer sheath assembly 22, the mid shaft assembly 21, the inner assembly 18, and the nose cone assembly 31, relative to the rail assembly 20. The buttons 550 may allow the user to increase or decrease the depth. The control device 504 of FIG. 49 may include buttons 552 that actuate deployment of the implant 70. For example, the buttons 552 may cause the motor to actuate the delivery apparatus to retract the outer sheath assembly 22 and the mid shaft assembly 21 to deploy the implant 70. The control device 504 of FIG. 49 may include buttons 554 that actuate movement of the nose cone assembly 31, to advance or retract the nose cone 28. Various configurations of control may be utilized to deflect the delivery apparatus or to perform operations of the delivery apparatus. The control signals from the control device 504 may be sent directly to the motor 500 for operation, or may be sent to the processor 536 for the processor 536 to operate the motor 500 to actuate at least a portion of the delivery apparatus. The configuration of the control device 504 may be varied in other embodiments. The control signals may be utilized to operate a deflection mechanism as disclosed herein.

Other embodiments of control devices that may be utilized include buttons, joysticks, touchpads, touch screens, knobs, or motion sensing devices, among other forms of control devices.

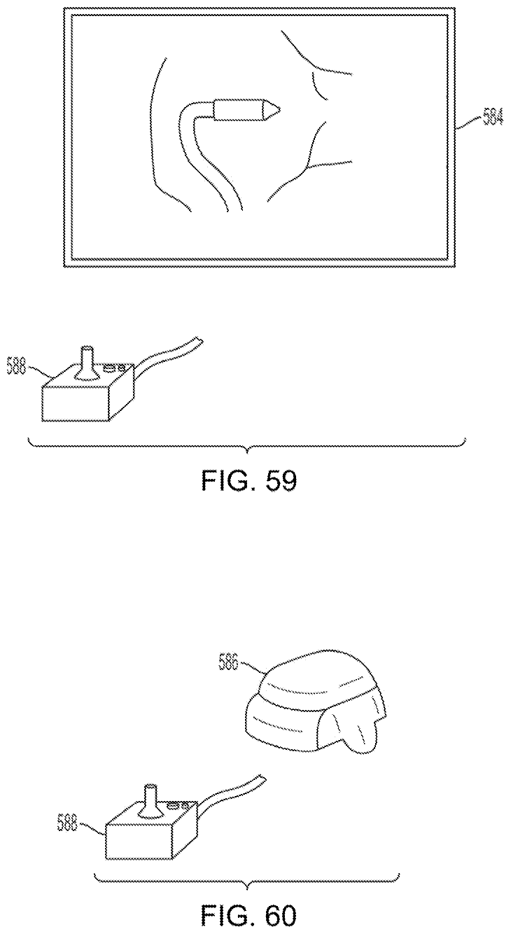

The system 10 may include an output device that may have various forms. The output device may be configured to provide an output to a user that may indicate a condition of the delivery apparatus or of the patient. The output device may be configured to provide an indicator of a condition of the delivery apparatus or of the patient. The output device may include lights that may illuminate to indicate a condition of the delivery apparatus or of the patient. The lights may illuminate to indicate the delivery apparatus has contacted or approached a surface of the patient's body (a condition of the delivery apparatus), or may illuminate to indicate a certain condition of the patient's body, such as a correct or incorrect pressure being sensed in the patient's body. Other forms of output devices may be utilized, including a haptic device, such as a vibrating actuator, which may indicate the condition of the delivery apparatus or of the patient. An output device may include the display screen of the touch screen. An output device may include a display screen 584 as shown in FIG. 59. An output device may include one or more of a display screen, a light, a speaker, or a haptic device, among other forms of output devices. Various forms of output devices may be utilized as desired. An indicator produced on the output device may include one or more of an image, data, a sound, a light, or a haptic signal. The output device may be configured to provide an indicator based on an output provided by the processor 536.

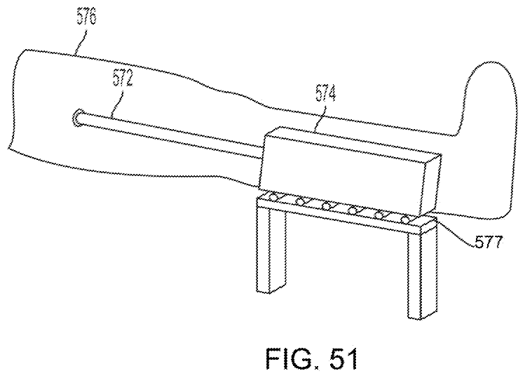

The actuation of the delivery apparatus by at least one motor may include a translation of the elongate shaft 12 and may include a translation of a housing at a proximal end of the elongate shaft 12. Axial translation of the delivery apparatus may be provided. FIG. 51, for example, illustrates a side perspective view of a delivery apparatus including an elongate shaft 572 and a housing 574. The delivery apparatus is being passed transfemorally into a patient's body 576. The elongate shaft 572 may be configured similarly as the elongate shaft 12. The housing 574 may be configured similarly as the housing forming the handle 15, however the housing 574 may not comprise a handle for grip by a user. Rather the housing 574 may include a motor, or may be configured to move along a motor driven rail 577 or other assembly that actuates axial movement of the delivery apparatus into the patient's body. The axial movement of the delivery apparatus may be controlled by a control device, which may be positioned proximate the housing 574 or may be located remote from the housing 574.

The motor 500 may be configured to actuate the delivery apparatus by selectively moving one or more of the outer sheath assembly 22, the mid shaft assembly 21, the inner assembly 18, the rail assembly 20, the assembly including the distal pull wires 138, the assembly including the proximal pull wires 140, and the nose cone assembly 31. The motor may be configured to perform any other method, or may be utilized with any embodiment disclosed herein, including the embodiments of FIGS. 13A-44 and 62A-64C.

In certain embodiments, the processor 536 may be utilized to automatically move the assemblies or other portions of the elongate shaft 12 to perform the operations of the delivery apparatus. For example, if a request is made to increase the depth of the elongate shaft 12 or deploy the implant 70, then the processor 536 may be configured to operate a program (which may be stored in memory 534) to control the motor 500 to move the corresponding assemblies or other portions of the elongate shaft 12. If a request is made that requires compensation of movement, then the processor 536 may be configured to operate a program (which may be stored in memory 534) to control the motor 500 to move the corresponding assemblies or other portions of the elongate shaft 12 to automatically perform such compensation. The processor 536 may be configured to operate the motor to move one of the assemblies to compensate for a motion of another of the assemblies. Particular movements and combinations of movements of the assemblies or other portion of the elongate shaft 12 may be programmed into the memory 534 and operated by the processor 536. As discussed above, the programmed movements may be based on data “learned” from previous procedures and, in particular, learned from previous procedures performed on patients with similar anatomies and/or other characteristics. The movements may be based on a machine learning algorithm utilizing data from past implantation procedures or from characteristics of the patient. Therefore, procedural steps performed successfully on patients with similar anatomies could be duplicated, thereby increasing the probability of a successful procedure on the current patient. The processor 536 may be configured to automatically operate the motor 500 to actuate a portion of the delivery apparatus in a desired manner.

The system 10 may include sensors that are configured to sense a condition of the delivery apparatus and may include sensors that are configured to sense a condition of the patient.

In certain embodiments, a sensor may be utilized to sense a condition of the delivery apparatus. The sensor may comprise a position sensor that may be utilized to determine the movement and/or position of one or more of the assemblies. For example, the position sensor may be configured to sense the amount that the motor 500 has moved the assembly to track the position and movement of the assembly. The motor 500 may be wired to track movement of the various assemblies and perform a desired movement (e.g., simultaneous movement of assemblies, or compensatory movement of one or more assemblies) based on the signal from the position sensor. In one embodiment, the signal from the position sensor may be provided to the processor 536 for the processor 536 to perform a desired movement. The signal from the position sensor may be a feedback signal to the processor 536. For example, the position sensor may sense that a portion of the elongate shaft 12 is moving in response to movement of another portion of the elongate shaft 12, and the processor 536 may operate the motor 500 to produce compensatory movement based on this signal. An indicator indicating a position of the delivery apparatus may be provided on an output device, as discussed herein. The indicator may be provided based on the position sensed by the position sensor.

A sensor may be utilized to sense a condition of the delivery apparatus in the form of a motor torque sensor. The sensor may be utilized to determine the amount of torque exerted by the motor 500. The motor torque sensor, for example, may be a current draw sensor able to sense the amount of current drawn by the motor 500. If the amount of torque exceeds a certain amount, the motor 500 may be configured to automatically shut off or reverse its operation or reduce torque. In one embodiment, the signal from the motor torque sensor may be provided to the processor 536 for the processor 536 to perform a desired movement. The signal from the motor torque sensor may be a feedback signal to the processor 536. For example, the processor 536 may operate the motor 500 to automatically shut off or reverse its operation or reduce torque based on this signal. An indicator indicating a torque of a motor of the delivery apparatus may be provided on an output device, as discussed herein. The indicator may be provided based on the torque sensed by the motor torque sensor.

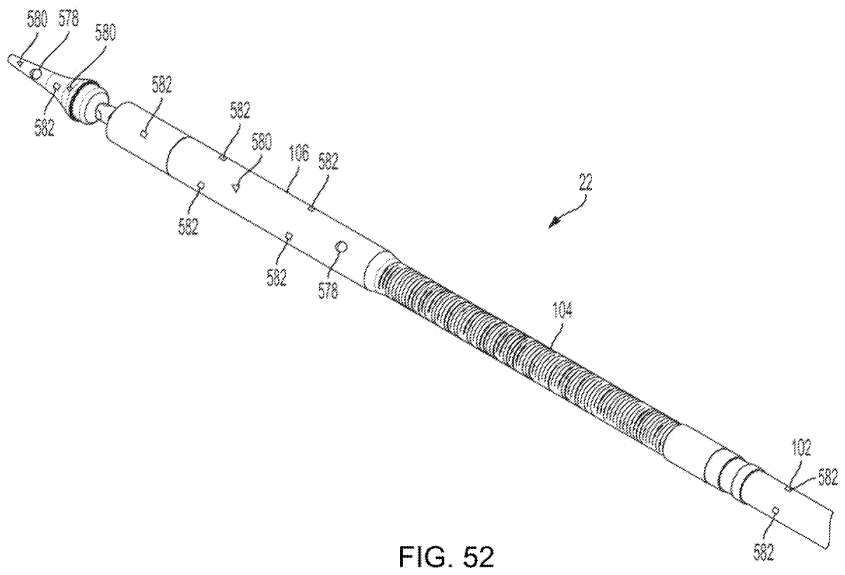

Referring to FIG. 52, sensors configured to sense a condition of the patient may be utilized. Such sensors may be positioned as desired on the delivery apparatus. Sensors configured to sense a condition of the patient may include ambient pressure sensors 578. Such pressure sensors 578 may be configured to sense a pressure, such as a fluid pressure, within the patient's body. The pressure sensors 578 may be utilized during and following delivery of the implant 70, to determine whether the deployed implant 70 is operating as desired following implantation, or to generally monitor a condition of the patient before and following implantation. In the embodiment shown in FIG. 52, a pressure sensor 578 may be positioned on the nose cone 28 and a pressure sensor may be positioned on the capsule 106 among other locations. With this particular configuration of pressure sensors 578, one pressure sensor may be positioned in the right ventricle during implantation of the implant 70, and one pressure sensor may be positioned in the right atrium during implantation. Thus, following implantation, the pressure gradient across the mitral valve can be determined. A signal from the pressure sensors 578 may be provided to an output device (such as output devices 568, 570, or other output device) for indication to the user. In one embodiment, the pressure sensed by the pressure sensors 578 may be utilized as feedback to the system 10, such as the processor 536, to actuate the delivery apparatus. For example, if an incorrect pressure is read, the processor 536 may actuate the delivery apparatus to redeploy the implant or perform another operation. In other embodiments, other positions of pressure sensors 578 and other pressure readings may be provided.

In one embodiment, a sensor configured to sense a condition of the delivery apparatus may include sensors configured to sense a spatial relationship between the delivery apparatus and a surface of the patient's body. Such a sensor may be positioned on the delivery apparatus. Such a sensor may include a contact sensor 580. A contact sensor 580 may comprise a force transducer or load cell, or other form of contact sensor 580 that is configured to sense a force applied to the delivery apparatus. As shown, a contact sensor 580 may be positioned in a variety of positions on the elongate shaft 12, including on the nose cone 28 or other locations (such as generally on the outer surface of the elongate shaft 12). A contact sensor 580 may be configured to provide a signal when the elongate shaft 12 contacts a portion of the patient's body. Such a signal may indicate the possibility of damage to the patient's body due to the elongate shaft 12. A signal from a contact sensor 580 may be provided to an output device (such as output devices 568, 570, or other output device) for indication to the user. In one embodiment, the contact sensed by the contact sensor 580 may be utilized as feedback to the system 10, such as the processor 536, to actuate the delivery apparatus. For example, if contact is sensed with a surface, then the processor 536 may actuate the delivery apparatus to move away from the surface or stop operation of the motor 500. In other embodiments, other positions of contact sensors 580 and other contact sensors may be provided.

In one embodiment, a sensor configured to sense a condition of the delivery apparatus may include a proximity sensor 582. The proximity sensor 582 may be configured to sense a spatial relationship between the delivery apparatus and a surface of the patient's body. Such a sensor may be positioned on the delivery apparatus. A proximity sensor 582 may comprise a device for sensing a distance to a portion of the patient's body, including use of ultrasound, or echo signals, or visual identification. As shown, a proximity sensor 582 may be positioned in a variety of positions on the elongate shaft 12, including on the nose cone 28 or other locations (such as generally on the outer surface of the elongate shaft 12). The proximity sensor 582 may be configured to provide a signal when the elongate shaft 12 approaches a portion of the patient's body, and may provide such a signal to an output device (such as output devices 568, 570, or other output device) for indication to the user. In one embodiment, the proximity sensed by the proximity sensor 582 may be utilized as feedback to the system 10, such as the processor 536, to actuate the delivery apparatus. For example, if proximity to a surface (e.g., an inner wall of blood vessel) is sensed, the processor 536 may actuate the delivery apparatus to move away from the surface or stop operation of the motor 500. As such, the delivery system could be advanced through the patient's vasculature without damaging an inner wall of a blood vessel. This “smart catheter” technology could provide a significant improvement over current “blind catheters.” For example, this technology could reduce or eliminate the possibility of vascular dissection, which is a significant and life-threatening risk with current delivery systems. Although embodiments have been described for sake of explanation, it will be understood that other positions of proximity sensors 582 and other proximity readings may be provided.

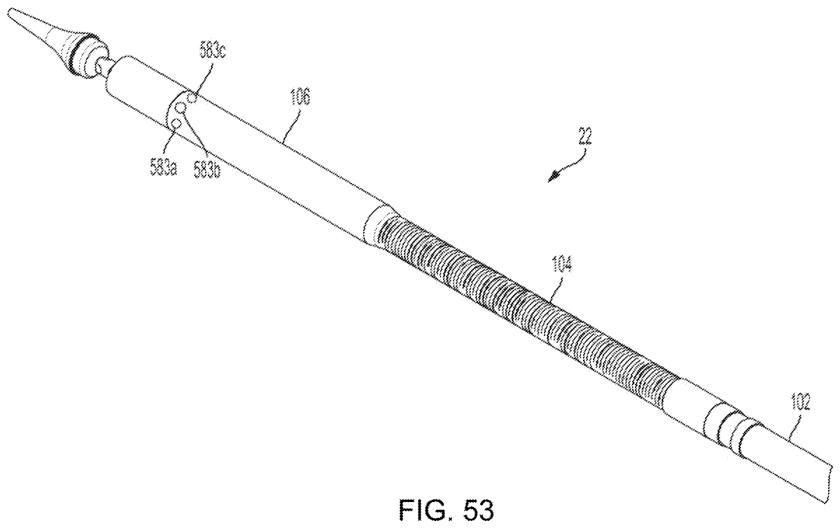

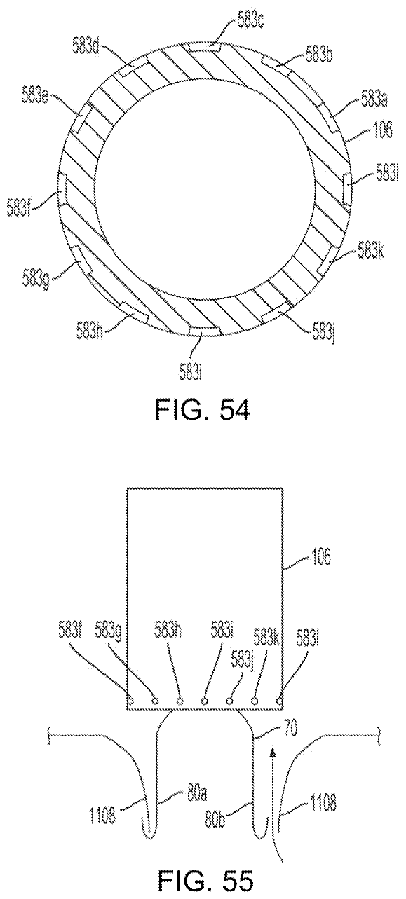

FIGS. 53-55 illustrate an embodiment of a sensor configured to sense a condition of the patient. The sensor comprises a flow sensor that may sense a fluid flow (e.g., blood flow) within the patient's body. A plurality of sensors 583a-l (as marked in FIG. 54) may be positioned on the delivery apparatus forming a spaced array of sensors 583a-l. The sensors 583a-l may be configured to sense a local fluid flow, such that the sensors 583a-l may sense a fluid flow in a local area in the body that is different from the fluid flow sensed by other sensors 583a-l. FIG. 53 illustrates a perspective view of the distal end of the elongate shaft 12, with sensors 583a—c visible on the capsule 106. FIG. 54 illustrates a cross sectional view of the capsule 106 showing the spaced array of sensors 583a-l. The sensors 583a-l may be positioned on the delivery apparatus to sense fluid flow at a location proximate the deployment location for the implant 70. Such a location may comprise the capsule 106 or another portion of the delivery apparatus.

FIG. 55 illustrates an exemplary operation of the sensors 583a-l. The implant 70 may be deployed to the tricuspid valve, with one distal anchor 80a capturing a leaflet 1108 and another distal anchor 80b failing to capture a leaflet 1108. The sensors 583k, 5831 may sense a flow of blood by the mis-captured leaflet 1108 and may provide a signal accordingly. The sensors 583a-l may be configured to sense a differential flow between the sensors 583f, 583g proximate the captured leaflet 1108 and the sensors 583k, 5831 proximate the mis-captured leaflet 1108. The flow sensors 583a-l may be configured to provide a signal when a flow is sensed, and may provide such a signal to an output device (such as output devices 568, 570, or other output device) for indication to the user. In one embodiment, the flow sensed by the flow sensors 583a-l may be utilized as feedback to the system 10, such as the processor 536, to actuate the delivery apparatus. For example, if flow is sensed indicated a mis-capture of a leaflet, then the processor 536 may actuate the delivery apparatus to redeploy the implant 70 or perform another operation. In other embodiments, other positions of flow sensors 583a-l and other flow readings may be provided.

The sensors that are configured to sense the condition of the delivery apparatus and the sensors that are configured to sense a condition of the patient may be coupled to the delivery apparatus. In certain embodiments, however, the sensors that are configured to sense the condition of the delivery apparatus and the sensors that are configured to sense a condition of the patient may not be coupled to the delivery apparatus and may be external to the patient's body.

The signals from the sensors that are configured to sense the condition of the delivery apparatus and the sensors that are configured to sense a condition of the patient, may be utilized in a variety of manners. In one embodiment, the signals may be provided as indicators on an output device (such as output devices 568, 570, or other output device) for indication to the user. For example, a condition of the delivery apparatus may be indicated to a user in a variety of forms, for example, an output device may include one or more of a display screen, a light, a speaker, or a haptic device, among other forms of output devices. An indicator produced on the output device may include one or more of an image, data, a sound, a light, or a haptic signal. The user may be able to act accordingly based on the indicator. For example, if an indicator indicates that the delivery apparatus has contacted a portion of the patient's body, then the user may act accordingly to move the delivery apparatus away from the body. A condition of the patient's body may similarly be indicated to a user in a variety of forms.

In embodiments, the signals from the sensors that are configured to sense the condition of the delivery apparatus and the sensors that are configured to sense a condition of the patient may be provided to the processor 536. The processor 536 may provide a variety of outputs based on the one or more of a condition of the patient's body or a condition of the delivery apparatus sensed by the one or more sensors. One such form of output includes a log of data for an implantation procedure with the delivery apparatus. Such a log of data may be stored in the memory 534. The data may be stored for later retrieval by a user for analysis, or may record a log of actions taken by the delivery apparatus. For example, the position sensor signals may be logged to record the movements of the delivery apparatus, among other forms of sensors signals.

The processor 536 may provide an output to an output device based on the condition of the patient's body or a condition of the delivery apparatus sensed by the one or more sensors. The output may result in an indicator on an output device (such as output devices 568, 570, or other output device) for indication to the user. For example, a condition of the delivery apparatus may be indicated to a user in a variety of forms, for example, an output device may include one or more of a display screen, a light, a speaker, or a haptic device, among other forms of output devices. The processor 536 may process the signals to produce a desired indicator to a user. For example, the sensors 583a-l may sense a flow of blood during deployment of the implant 70, and the processor 536 may process these signals to provide an indicator to a user that leaflet mis-capture has occurred.

The processor 536 may provide an output that comprises a control of the motor 500 based on the condition of the patient's body or a condition of the delivery apparatus sensed by the one or more sensors. The processor 536 may be configured to operate the motor 500 to actuate the delivery apparatus based on a signal from the sensors. The signal from the sensors may comprise feedback signals that are input to the processor 536 for the processor to control operation of the motor 500. For example, a signal from a contact sensor 580 or a proximity sensor 582 may be provided to the processor 536 as feedback that the delivery apparatus has contacted or is proximate a surface of the patient's body. The processor 536 accordingly may provide an output that operates the motor 500 to avoid or retract from the surface of the patient's body. A signal from the flow sensors 583a-l may cause the processor 536 to provide an output to the motor 500 to redeploy the implant 70 or move the portion of the delivery apparatus to recapture the leaflet 1108. A signal from a position sensor may provide feedback to the processor 536 regarding whether the delivery apparatus is performing the correct movements, and the processor 536 may operate the motor 500 to perform corrective movements if desired (e.g., deflect the elongate shaft 12 if needed). The processor 536 may be programmed to automatically respond and produce outputs based on the condition of the patient's body or a condition of the delivery apparatus sensed by the one or more sensors. The programming for the processor 536 may be stored in the memory 534 and operated by the processor 536.

The delivery system can be used in a method for percutaneous delivery of a replacement tricuspid valve to treat patients with moderate to severe tricuspid regurgitation. However, it will be understood that the delivery systems described herein can be used as part of other methods as well, such as implants for repair of valves and delivery of implants to other heart valves and delivery of other implants.

In one embodiment, a method may include extending a delivery apparatus within a portion of the patient's body to deliver an implant to a body location. The delivery system 10 can be placed in the ipsilateral femoral vein and advanced toward the right atrium. Accordingly, it can be advantageous for a user to be able to steer the delivery system 10 through the complex areas of the heart in order to position a replacement tricuspid valve in line with the native tricuspid valve. This task can be performed with or without the use of a guide wire. The distal end of the delivery system can be advanced towards or into the left atrium. The motor 500 may then be operated to actuate the rail assembly 20 or the deflection mechanism to target the distal end of the delivery system 10 to the appropriate area. The motor 500 may be operated by a processor 536 as discussed herein. The motor 500 may be operated to create a variety of bends in the rail assembly 20 and deflect the elongate shaft 12 in a variety of manners to place the implant in the desired location for implantation.

The operation of the motor 500 may be operated by a processor 536. A user may provide input to the processor 536 with a control device 504.

Further the sensors discussed herein may be utilized in certain embodiments. The delivery apparatus may include one or more sensors coupled to the delivery apparatus and configured to sense one or more of a condition of the patient's body or a condition of the delivery apparatus. The processor 536 may be configured to provide an output based on the one or more of a condition of the patient's body or a condition of the delivery apparatus sensed by the one or more sensors. For example, the processor may cause at least a portion of the delivery apparatus to avoid or retract from a surface of the patient's body based on a condition of the delivery apparatus.

The use of a processor, one or more sensors, and/or one or more motors with a delivery system, as disclosed herein, may be configured to perform any other method, or may be utilized with any embodiment disclosed herein, including the embodiments of FIGS. 13A-44 and 62A-64C.

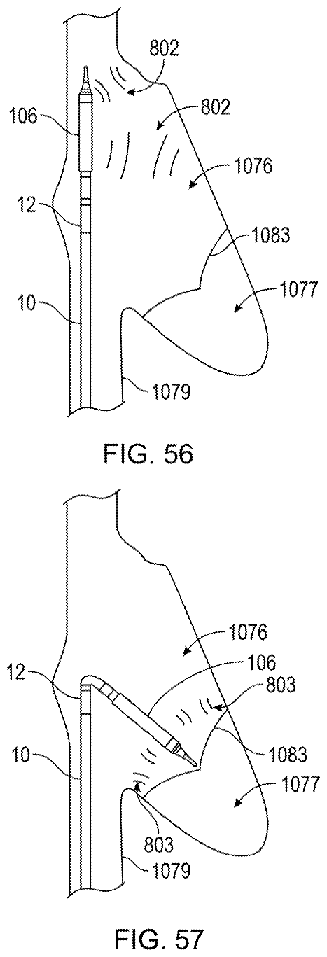

In embodiments, the delivery system 10 can be used in a method for percutaneous delivery of a replacement tricuspid valve that may be used to treat patients with moderate to severe tricuspid regurgitation. Such a method may utilize any of the systems or devices disclosed herein. Referring to FIG. 56, for example, the delivery apparatus may be extended within a portion of a patient's body to deliver an implant to a body location. The portion of the patient's body may be the right atrium 1076 and the body location for delivering the implant may be the native tricuspid heart valve 1083. The delivery apparatus may be extended within a portion of the patient's body in a similar manner as disclosed herein, for example, the delivery apparatus can be placed in the ipsilateral femoral vein and advanced towards the right atrium 1076. Other entry methods may be utilized as desired.

The delivery apparatus may be extended within the inferior vena cava 1079 into the right atrium 1076. One or more motors, which may be operated by a processor 536 as discussed herein, may be utilized to extend the delivery apparatus into the right atrium 1076.

The delivery apparatus may be steered through the complex areas of the heart in order to position a replacement tricuspid valve in line with the native tricuspid valve. The motor 500 may be operated to actuate the rail assembly 20 to target the distal end of the delivery apparatus to the appropriate area. For example, the motor 500 may be utilized to steer the rail assembly 20 to the desired orientation relative to the tricuspid heart valve 1083. The motor 500 may be operated by a processor 536 as discussed herein. The rail assembly 20 may form one or more bends such that the distal end of the delivery apparatus is oriented coaxial with the native tricuspid heart valve 1083.

FIG. 57, for example, shows that the delivery apparatus has been deflected within the right atrium 1076 towards the native tricuspid heart valve 1083. One or more bends may be formed within the right atrium 1076 and/or the inferior vena cava 1079. Once the implant 70 is positioned coaxial with the native tricuspid heart valve 1083, the outer sheath assembly 22, mid shaft assembly 21, inner assembly 18, and nose cone assembly 31 can together be advanced (e.g., using the motor 500) distally relative to the rail assembly 20 towards the right ventricle 1077. The depth of the elongate shaft 12 may be varied by the operation of the motor 500 disclosed herein, which may be operated by a processor 536. The proximal/distal translation of the other assemblies over the rail assembly 20 allows for ventricular-atrial motion. Further, a deflection mechanism as disclosed herein may be utilized. Other features from other embodiments disclosed herein may be utilized as desired.

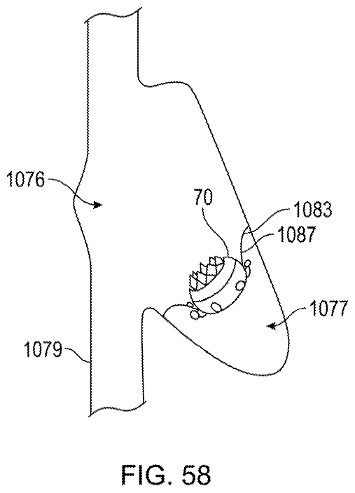

The depth of the elongate shaft 12 may be varied until the capsule 106 is positioned in the desired location relative to the native tricuspid heart valve 1083. The distal end 303 of the implant 70, and specifically the distal anchors 80, may be restrained within the capsule 106 of the outer sheath assembly 22, thus preventing expansion of the implant 70. Similar to what is shown in FIG. 2A, the distal anchors 80 can extend distally when positioned in the capsule. The proximal end 301 of the implant 70 is restrained within the capsule 106 and within a portion of the inner retention member 40 and thus is generally constrained between the capsule 106 and the inner retention member 40. The implant 70 may then be deployed to the native tricuspid heart valve 1082. FIG. 58, for example, illustrates the implant 70 deployed to the native tricuspid heart valve 1082. The distal anchors of the implant 70 extend over the leaflets 1087 of the tricuspid heart valve 1083. The delivery apparatus may then be withdrawn from the patient's right atrium 1076.

The method may utilize the systems and devices disclosed herein. For example, the motor 500 may deflect a portion of the delivery apparatus or deploy the implant to the body location. The motor may operate a deflection mechanism as disclosed herein, or other feature of an embodiment disclosed herein, including controlling operation of the embodiments of FIGS. 13A-44 and 62A-64C. The operation of the motor 500 may be operated by a processor 536. A user may provide input to the processor 536 with a control device 504. The system 10 can be positioned through the use of the steering mechanisms discussed herein or other techniques. The delivery system 10 can be advanced by the user manually moving the handle 15 in an axial direction. In some embodiments, the delivery system 10 can be placed into a stand while operating the handle 15 controls.

The delivery apparatus may be utilized in the form shown in FIG. 1, or other forms of delivery apparatuses may be utilized, for example, delivery apparatuses configured for delivery of an implant to the native tricuspid valve.

In other embodiments, other methods of delivering the implant to the native tricuspid heart valve may be utilized, for example, a transapical, transseptal, or other method may be utilized.

Other locations for valve implant may include the aortic or pulmonary valve, and other valves of a patient's body. Other forms of implants may be delivered to other body locations as desired.

In some embodiments, the implant 70 can be delivered under fluoroscopy so that a user can view certain reference points for proper positioning of the implant 70. Further, echocardiography can be used for proper positioning of the implant 70.

In one embodiment, the proximity sensor 582 may be configured to provide a model of the interior of the patient's body and the spatial relationship of the elongate shaft 12 from surfaces of the patient's body. Such a model may be provided on output devices 584, 586 shown in FIGS. 59 and 60 as display screens (on a monitor and on a virtual reality or augmented reality display). Such a model may also be provided by other sensors positioned external to the patient's body if desired. Such a model may be a two-dimensional map or three-dimensional map of the patient's body for view by a user, and for use by the processor 536 as feedback to navigate through the patient's body and deliver an implant 70 to the desired location.

FIG. 59 illustrates an embodiment in which operation of the delivery apparatus may occur remotely by a user. The user may utilize a control device 588 such as a joystick or other form of control device to control movement of the delivery apparatus and elongate shaft 12. The control device 588 may be configured to sense motion of the control device to control the delivery apparatus. The user may view the position of the elongate shaft 12 on an output device 584 in the form of a display screen. The position may be provided in a variety of manners, including external sensing of the position via sensors using fluoroscopy or echocardiography. The position may also be provided via an image produced by signals from proximity sensors of the elongate shaft 12. The proximity sensors may be configured to produce an image of the spatial relationship between the elongate shaft 12 and the surfaces of the patient's body. A configuration including a motor for axial movement of the elongate shaft 12, as shown in FIG. 54, may be utilized as well for remote control of the procedure.

FIG. 60 illustrates an embodiment in which the output device 586 is in the form of a display screen on a virtual reality or augmented reality display. The display may include a helmet (or other headset that allows for enhanced visualization) for wear by the user, wherein the user is able to move his or her head to alter the perspective of the view provided by the display screen. Similar to the embodiment discussed with respect to FIG. 59, the position of the elongate shaft 12 and portions of the patient's heart seen in the output device 586 may be provided in a variety of manners, including external sensing of the position via fluoroscopy or echocardiography. The position may also be provided via an image produced by signals from proximity sensors of the elongate shaft 12. The proximity sensors may be configured to produce an image of the spatial relationship between the elongate shaft 12 and the surfaces of the patient's body. A configuration including a motor for axial movement of the elongate shaft 12, as shown in FIG. 51, may be utilized as well for remote control of the procedure.

In an exemplary method, a user (e.g., clinician) may provide input, which may be assisted by use of the components disclosed herein (e.g., the processor, motor, and one or more sensors, among other components). In embodiments, however, an implantation procedure may occur autonomously (i.e., adapts to environment during operation). The processor may perform autonomous control of the delivery apparatus to perform the implantation procedure. A user may provide some input during the procedure, such that the procedure may occur semi-autonomously. As such, a method may occur autonomously or semi-autonomously (or at least semi-autonomously). Other autonomous procedures may include autonomously performing the methods disclosed in regard to the embodiments of FIGS. 13A-44 and 62A-64C.

A method may include extending a delivery apparatus within a portion of a patient's body to deliver an implant to a body location. The delivery apparatus may be configured similarly as any embodiment of delivery apparatus disclosed herein. The delivery apparatus may be extended within a portion of the patient's body as disclosed herein. The implant may be configured similarly as any implant disclosed herein, and the body location may comprise any location disclosed herein.

The delivery apparatus may be extended within the portion of the patient's body by way of a motor advancing the delivery apparatus, such as the elongate shaft of the delivery apparatus within the patient's body. The motor may be controlled by the processor 536. For example, a motor driven rail 577, or other assembly that actuates axial movement of the delivery apparatus into the patient's body may be utilized. In other embodiments, other methods may be utilized to extend the delivery apparatus within the portion of the patient's body.

The processor 536 may operate a program to actuate the delivery apparatus. The processor 536 may be programmed with a sequence of movements to actuate the delivery apparatus to the desired location and for the desired deployment operation. For example, the processor 536 may be configured to determine the desired delivery location and the path and orientation to be followed to reach the desired delivery location based on external sensing of the position via fluoroscopy or echocardiography and/or the position being determined via signals from proximity sensors of the elongate shaft 12. The programmed sequence of movement may be provided based on the geometry of the path to the desired implant location, and the orientation of the desired implant location. The movement and deployment of the delivery apparatus may be preprogrammed into the processor 536 and may be individualized based on the particular path to the desired location in the patient's body to be followed. In certain embodiments, a machine learning algorithm may be utilized by the processor 536 to control actuation of the delivery apparatus. For example, the path and orientation also be supplemented by data from previous procedures on patients with similar characteristics. The processor 536 and programming may be utilized to extend the delivery apparatus within a portion of the patient's body as disclosed.

The processor 536 may continue to follow the program, and may receive signals from one or more sensors. The processor 536 may receive feedback from sensors (as discussed herein) that cause the processor 536 to produce outputs. The signals from the sensors may be utilized by the processor 536 in a similar manner as disclosed herein. For example, the processor 536 may be configured to produce a log of data. The processor 536 may be configured to produce an indicator. The indicator may be provided for a user to determine whether to intervene in a procedure. For example, if a user (e.g., a clinician) receives an indicator that the autonomously operated delivery apparatus has contacted a surface or has improperly deployed an implant, then the user may intervene to attempt to correct such actuation.

The processor 536 may be configured to produce actuation of the delivery apparatus. The actuation may be provided for the processor 536 to correct the path and operation with minimal or no human interaction using feedback from sensors as discussed herein, to complete the procedure. For example, if the position sensor indicates the delivery apparatus is straying from the intended path, the processor 536 may automatically adjust the path. If the proximity sensor indicated the delivery apparatus is approaching a surface, then the processor 536 may automatically adjust the path. The processor 536 may be used to navigate to any desired location for delivery of the implant. Any of the sensors and feedback operations from the sensors disclosed herein may be utilized in such a method. In certain embodiments, a user may provide some input during the procedure to correct the procedure or otherwise provide input to control the procedure.

The actuation produced by the processor 536 may be based on a machine learning algorithm utilizing data from past implantation procedures or from characteristics of the patient. The actuation may be based on data “learned” from previous procedures and, in particular, learned from previous procedures performed on patients with similar anatomies and/or other characteristics. Therefore, procedural steps performed successfully on patients with similar anatomies could be duplicated, thereby increasing the probability of a successful procedure on the current patient. A machine learning algorithm may be utilized by the processor 536 to control actuation of the delivery apparatus.

The processor 536 may be configured to operate the motor 500 to produce the desired actuation of the delivery apparatus. The processor 536 may be configured to automatically operate the motor to deflect the delivery apparatus to the desired body location. The processor 536 may be configured to automatically operate the motor to deflect the delivery apparatus in at least two planes. The processor 536 may be configured to automatically deploy the implant 70 to the desired location and complete the delivery procedure. The processor 536 may be configured to complete the delivery procedure in certain embodiments without control or intervention by a user. The processor 536 may be configured to provide such a confirmation of implantation as an indicator on an output device, so that the user is notified that the implant has been implanted.

The methods may be utilized for replacement or repair of a heart valve within a patient's body. The heart valve may comprise one or more of an aortic heart valve, a mitral heart valve, a tricuspid heart valve, or a pulmonary heart valve. Other valves or body locations for implantation may be treated in other embodiments.

FIG. 61 illustrates an embodiment of a delivery apparatus configured similarly as the apparatus shown in FIG. 46, however, multiple motors 502 may be utilized to control actuation of the delivery apparatus. The multiple motors 502, for example, may each be configured to engage respective adaptors 590, 592, 594 configured to actuate portions of the delivery apparatus. The motors 502 may be configured to perform linear movement of the adaptors 590, 592, 594 to cause actuation of the delivery apparatus. Further, in the embodiment of FIG. 61, the processor, memory, and input device and output device of FIG. 61 may be provided on a printed circuit board 596 positioned within the handle. A power source 598 such as a battery pack or other form of power source may also be utilized within the handle. The embodiment of FIG. 61 may comprise a self-contained handle unit including a processor for performing a delivery procedure and receiving feedback from sensors, as well as performing data logging if desired.

The motors disclosed herein may comprise a variety of forms of motors, including electromagnetic, stepper, hydraulic, piezoelectric, among others. The methods, systems, and apparatuses disclosed herein in regard to FIGS. 45-61 may be utilized with any embodiment disclosed herein. For example, actuation and control of any of the systems, apparatuses, or methods of any of the embodiments of FIG. 13A-44 or 62A-64C may occur under the operation of the systems, apparatuses or methods of the embodiments of FIGS. 45-61.

Although many of the systems and methods disclosed herein have been discussed in regard to implantation of a prosthetic tricuspid valve implant, it is understood that the systems and methods may be utilized to deliver a variety of implants, including implants for repair of a heart valve. For example, other types of heart valve implants that may be utilized than are shown herein, among other types of implants (e.g., aortic valve implants and other repair implants).

The methods and systems disclosed herein may in certain embodiments not be limited to delivery of implants, but may extend to any medical intervention or insertion into a patient's body, which may include performing a medical procedure within the body. The methods and systems disclosed herein may be utilized in general use of a catheter as desired. For example, the handle shown in FIG. 61 and components disclosed therein may comprise a general catheter handle in certain embodiments. Further, the configuration of the delivery apparatus may be modified in other embodiments. For example, for an aortic valve delivery apparatus, the configuration of the implant retention area and other features of the delivery apparatus may be modified.

Although many of the embodiments herein are discussed in regard to a replacement tricuspid valve, the deflection mechanisms and other embodiments disclosed herein may be utilized for a variety of other implementations including delivery of mitral replacement valves, or aortic or pulmonary valves, or for valve repair procedures, including tricuspid or mitral valve repair or aortic or pulmonary valve repair.

From the foregoing description, it will be appreciated that an inventive product and approaches for implant delivery systems are disclosed. While several components, techniques and aspects have been described with a certain degree of particularity, it is manifest that many changes can be made in the specific designs, constructions and methodology herein above described without departing from the spirit and scope of this disclosure.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

1.-184. (canceled)

185. A delivery system for replacing the function of a native heart valve, the delivery system comprising:
a prosthetic heart valve implant;
an elongate shaft having:
a distal end portion,
an implant retention area for retaining the prosthetic heart valve implant in a compressed configuration,
a first deflectable bend portion located proximal to the implant retention area and configured to deflect the implant retention area and the distal end portion in a first direction,
a second deflectable bend portion located proximal to the first deflectable bend portion and configured to deflect the implant retention area and the distal end portion in a second direction, wherein deflection in the second direction is in a different plane than the deflection in the first direction; and
a sheath positioned over the elongate shaft, the sheath configured to deflect a portion of the elongate shaft that is proximal to both the first and second deflectable bend portions in a third direction that is opposed to the first direction.

186. The delivery system of claim 185, wherein the third direction is coplanar with the first direction.

187. The delivery system of claim 185, wherein the second direction is perpendicular to the first direction.

188. The delivery system of claim 185, wherein the sheath is configured to deflect the portion of the elongate shaft in the third direction to increase height of the distal end portion from a deployment site of the prosthetic heart valve implant.

189. The delivery system of claim 185, wherein the sheath is configured to rotate around the elongate shaft to vary a plane of the third direction.

190. The delivery system of claim 189, wherein:
the first deflectable bend portion is configured to defect the implant retention area and the distal end portion in a first plane,
the second deflectable bend portion is configured to deflect the implant retention area and the distal end portion in a second plane that is different than the first plane, and
the sheath is configured to deflect the portion of the elongate shaft that is proximal to both the first and second deflectable bend portions in a third plane that is different than the first plane and the second plane.

191. The delivery system of claim 185, wherein the elongate shaft includes:
an inner shaft,
a rail shaft extending over the inner shaft and including the first and second deflectable bend portions, and
an outer sheath extending over the rail shaft and configured to slide relative to the rail shaft to vary a distance of the implant retention area from the rail shaft.

192. The delivery system of claim 185, further comprising a first pull tether and a second pull tether, wherein the first pull tether is configured to actuate the first deflectable bend portion and the second pull tether is configured to actuate the second deflectable bend portion.

193. The delivery system of claim 185, wherein the elongate shaft is sized for transfemoral advancement into a patient's body.

194. The delivery system of claim 185, wherein the elongate shaft has an axial dimension and includes a port for the prosthetic heart valve implant to be deployed from the implant retention area in a direction transverse to the axial dimension.

195. The delivery system of claim 185, wherein the elongate shaft is configured to bend more than 180 degrees to form a loop.

196. The delivery system of claim 185, wherein the elongate shaft includes:
a capsule surrounding the implant retention area, and
a hinge coupling the capsule to a portion of the elongate shaft, the capsule configured to rotate about the hinge.

197. The delivery system of claim 185, further comprising a port positioned on the prosthetic heart valve implant and configured to receive a diagnostic or therapeutic device.

198. The delivery system of claim 185, wherein the prosthetic heart valve implant comprises a replacement tricuspid heart valve.

199. The delivery system of claim 185, wherein the prosthetic heart valve implant comprises a replacement heart valve including prosthetic heart valve leaflets.

200. A delivery system for replacing the function of a native heart valve, the delivery system comprising:
a prosthetic heart valve implant;
an elongate shaft having:
a distal end portion,
an implant retention area for retaining the prosthetic heart valve implant in a compressed configuration,
a first deflectable bend portion located proximal to the implant retention area and configured to deflect the implant retention area and the distal end portion in a first direction,
a second deflectable bend portion located proximal to the first deflectable bend portion and configured to deflect the implant retention area and the distal end portion in a second direction, wherein deflection in the second direction is in a different plane than the deflection in the first direction; and
a deflection mechanism configured to deflect a portion of the elongate shaft that is proximal to both the first and second deflectable bend portions in a third direction that is opposed to the first direction.

201. The delivery system of claim 200, further comprising a first pull tether, a second pull tether, and a third pull tether, wherein the first pull tether is configured to actuate the first deflectable bend portion, the second pull tether is configured to actuate the second deflectable bend portion, and the third pull tether is configured to actuate the deflection mechanism.

202. The delivery system of claim 201, wherein the third pull tether includes a distal end coupled to the elongate shaft proximal of the second deflectable bend portion.

203. The delivery system of claim 200, wherein the elongate shaft includes:
an inner shaft,
a rail shaft extending over the inner shaft and having one or more cuts thereon, and
an outer sheath extending over the rail shaft; and
the deflection mechanism includes a stopper on the rail shaft and a stopper on the inner shaft that is configured to apply a force to the stopper on the rail shaft to cause a deflection of the rail shaft at the one or more cuts.

204. The delivery system of claim 203, wherein the inner shaft is configured to be drawn proximally to cause the stopper on the inner shaft to apply the force to the stopper on the rail shaft.
